SEQUENCE LISTING (353 sequences)

SEQ ID NO: 1 - DNA, 20 bp, Artificial Sequence
  ErbB1 left primer
cagcgctaccttgtcattca

SEQ ID NO: 2 - DNA, 20 bp, Artificial Sequence
  ErbB1 right primer
tgcactcagagagctcagga

SEQ ID NO: 3 - DNA, 20 bp, Artificial Sequence
  ErbB1 hybridization oligonucleotide
gaatgcatttgccaagtcct

SEQ ID NO: 4 - DNA, 20 bp, Artificial Sequence
  ErbB1 left primer
gggctcacagcaaacttctc

SEQ ID NO: 5 - DNA, 20 bp, Artificial Sequence
  ErbB1 right primer
aagccagactcgctcatgtt

SEQ ID NO: 6 - DNA, 20 bp, Artificial Sequence
  ErbB1 hybridization oligonucleotide
acacacacacacacacaccg

SEQ ID NO: 7 - DNA, 20 bp, Artificial Sequence
  ErbB1 left primer
ggctcacagcaaacttctcc

SEQ ID NO: 8 - DNA, 20 bp, Artificial Sequence
  ErbB1 right primer
aagccagactcgctcatgtt

SEQ ID NO: 9 - DNA, 20 bp, Artificial Sequence
  ErbB1 hybridization oligonucleotide
acacacacacacacacaccg

SEQ ID NO: 10 - DNA, 20 bp, Artificial Sequence
  ErbB1 left primer
acttgacaggggaaacatgc

SEQ ID NO: 11 - DNA, 20 bp, Artificial Sequence
  ErbB1 right primer
caaggtctgggaaccactgt

SEQ ID NO: 12 - DNA, 20 bp, Artificial Sequence
  ErbB1 hybridization oligonucleotide
ttgcacaattccaaccttga

SEQ ID NO: 13 - DNA, 20 bp, Artificial Sequence
  ErbB1 left primer
ggctcacagcaaacttctcc

SEQ ID NO: 14 - DNA, 20 bp, Artificial Sequence
  ErbB1 right primer
gcatgtttcccctgtcaagt

SEQ ID NO: 15 - DNA, 20 bp, Artificial Sequence
  ErbB1 hybridization oligonucleotide
acacacacacacacacaccg

SEQ ID NO: 16 - DNA, 20 bp, Artificial Sequence
  ErbB1 left primer
gggctcacagcaaacttctc

SEQ ID NO: 17 - DNA, 20 bp, Artificial Sequence
  ErbB1 right primer
gcatgtttcccctgtcaagt

SEQ ID NO: 18 - DNA, 20 bp, Artificial Sequence
  ErbB1 hybridization oligonucleotide
acacacacacacacacaccg

SEQ ID NO: 19 - DNA, 20 bp, Artificial Sequence
  ErbB2/HER2 left primer
ccataacacccacctctgct

SEQ ID NO: 20 - DNA, 20 bp, Artificial Sequence
  ErbB2/HER2 right primer
actggctgcagttgacacac

SEQ ID NO: 21 - DNA, 20 bp, Artificial Sequence
  ErbB2/HER2 hybridization oligonucleotide
accaagctctgctccacact

SEQ ID NO: 22 - DNA, 20 bp, Artificial Sequence
  ErbB2/HER2 left primer
acacagcggtgtgagaagtg

SEQ ID NO: 23 - DNA, 14 bp, Artificial Sequence
  ErbB2/HER2 right primer
aggccaggggagag

SEQ ID NO: 24 - DNA, 20 bp, Artificial Sequence
  ErbB2/HER2 hybridization oligonucleotide
tcagaccctcttgggaccta

SEQ ID NO: 25 - DNA, 20 bp, Artificial Sequence
  ErbB2/HER2 left primer
gcctccacttcaaccacagt

SEQ ID NO: 26 - DNA, 17 bp, Artificial Sequence
  ErbB2/HER2 right primer
cccacgtccgaaaggta

SEQ ID NO: 27 - DNA, 20 bp, Artificial Sequence
  ErbB2/HER2 hybridization oligonucleotide
tgtgactgcctgtccctaca

SEQ ID NO: 28 - DNA, 20 bp, Artificial Sequence
  ErbB2/HER2 left primer
cccagctctttgaggacaac

SEQ ID NO: 29 - DNA, 20 bp, Artificial Sequence
  ErbB2/HER2 right primer
agccagctggttgttcttgt

SEQ ID NO: 30 - DNA, 20 bp, Artificial Sequence
  ErbB2/HER2 hybridization oligonucleotide
agcttcgaagcctcacagag

SEQ ID NO: 31 - DNA, 20 bp, Artificial Sequence
  ErbB2/HER2 left primer
tggggagagagttctgagga

SEQ ID NO: 32 - DNA, 20 bp, Artificial Sequence
  ErbB2/HER2 right primer
acagatgccactgtggttga

SEQ ID NO: 33 - DNA, 19 bp, Artificial Sequence
  ErbB2/HER2 hybridization oligonucleotide
gactgctgccatgagcagt

SEQ ID NO: 34 - DNA, 20 bp, Artificial Sequence
  ErbB2/HER2 left primer
cccagctctttgaggacaac

SEQ ID NO: 35 - DNA, 20 bp, Artificial Sequence
  ErbB2/HER2 right primer
ggatcaagacccctcctttc

SEQ ID NO: 36 - DNA, 20 bp, Artificial Sequence
  ErbB2/HER2 hybridization oligonucleotide
agcttcgaagcctcacagag

SEQ ID NO: 37 - DNA, 20 bp, Artificial Sequence
  ErbB2/HER2 left primer
ccataacacccacctctgct

SEQ ID NO: 38 - DNA, 20 bp, Artificial Sequence
  ErbB2/HER2 right primer
actggctgcagttgacacac

SEQ ID NO: 39 - DNA, 20 bp, Artificial Sequence
  ErbB2/HER2 hybridization oligonucleotide
accaagctctgctccacact

SEQ ID NO: 40 - DNA, 20 bp, Artificial Sequence
  ErbB2/HER2 left primer
ccatctgcaccattgatgtc

SEQ ID NO: 41 - DNA, 17 bp, Artificial Sequence
  HER2 right primer
gagcggaaggtgctgtc

SEQ ID NO: 42 - DNA, 20 bp, Artificial Sequence
  HER2 hybridization oligonucleotide
cgggagttggtgtctgaatt

SEQ ID NO: 43 - DNA, 20 bp, Artificial Sequence
  ErbB2/HER2 left primer
ccctcatccaccataacacc

SEQ ID NO: 44 - DNA, 20 bp, Artificial Sequence
  ErbB2/HER2 right primer
actggctgcagttgacacac

SEQ ID NO: 45 - DNA, 20 bp, Artificial Sequence
  ErbB2/HER2 hybridization oligonucleotide
accaagctctgctccacact

SEQ ID NO: 46 - DNA, 20 bp, Artificial Sequence
  ErbB2/HER2 left primer
cgcttttggcacagtctaca

SEQ ID NO: 47 - DNA, 20 bp, Artificial Sequence
  ErbB2/HER2 right primer
tcccggacatggtctaagag

SEQ ID NO: 48 - DNA, 20 bp, Artificial Sequence
  ErbB2/HER2 hybridization oligonucleotide
aattccagtggccatcaaag

SEQ ID NO: 49 - DNA, 20 bp, Artificial Sequence
  ErbB2/HER2 left primer
aattccagtggccatcaaag

SEQ ID NO: 50 - DNA, 20 bp, Artificial Sequence
  ErbB2/HER2 right primer
tcccggacatggtctaagag

SEQ ID NO: 51 - DNA, 20 bp, Artificial Sequence
  ErbB2/HER2 hybridization oligonucleotide
ggtgacacagcttatgccct

SEQ ID NO: 52 - DNA, 20 bp, Artificial Sequence
  ErbB2/HER2 left primer
ccataacacccacctctgct

SEQ ID NO: 53 - DNA, 20 bp, Artificial Sequence
  ErbB2/HER2 right primer
actggctgcagttgacacac

SEQ ID NO: 54 - DNA, 20 bp, Artificial Sequence
  ErbB2/HER2 hybridization oligonucleotide
accaagctctgctccacact

SEQ ID NO: 55 - DNA, 20 bp, Artificial Sequence
  ErbB2/HER2 left primer
ccctcatccaccataacacc

SEQ ID NO: 56 - DNA, 20 bp, Artificial Sequence
  ErbB2/HER2 right primer
actggctgcagttgacacac

SEQ ID NO: 57 - DNA, 20 bp, Artificial Sequence
  ErbB2/HER2 hybridization oligonucleotide
accaagctctgctccacact

SEQ ID NO: 58 - DNA, 20 bp, Artificial Sequence
  ErbB2/HER2 left primer
cgcttttggcacagtctaca

SEQ ID NO: 59 - DNA, 20 bp, Artificial Sequence
  ErbB2/HER2 right primer
tcccggacatggtctaagag

SEQ ID NO: 60 - DNA, 20 bp, Artificial Sequence
  ErbB2/HER2 hybridization oligonucleotide
aattccagtggccatcaaag

SEQ ID NO: 61 - DNA, 20 bp, Artificial Sequence
  ErbB2/HER2 left primer
aattccagtggccatcaaag

SEQ ID NO: 62 - DNA, 20 bp, Artificial Sequence
  ErbB2/HER2 right primer
tcccggacatggtctaagag

SEQ ID NO: 63 - DNA, 20 bp, Artificial Sequence
  ErbB2/HER2 hybridization oligonucleotide
ggtgacacagcttatgccct

SEQ ID NO: 64 - DNA, 20 bp, Artificial Sequence
  ErbB2/HER2 left primer
aattccagtggccatcaaag

SEQ ID NO: 65 - DNA, 20 bp, Artificial Sequence
  ErbB2/HER2 right primer
tttcccggacatggtctaag

SEQ ID NO: 66 - DNA, 20 bp, Artificial Sequence
  ErbB2/HER2 hybridization oligonucleotide
ggtgacacagcttatgccct

SEQ ID NO: 67 - DNA, 19 bp, Artificial Sequence
  ErbB3 left primer
gagcccagaggagaagact

SEQ ID NO: 68 - DNA, 20 bp, Artificial Sequence
  ErbB3 right primer
tctgatgcgacagacactcc

SEQ ID NO: 69 - DNA, 20 bp, Artificial Sequence
  ErbB3 hybridization oligonucleotide
gagtctgagtgttcggaggg

SEQ ID NO: 70 - DNA, 20 bp, Artificial Sequence
  ErbB3 left primer
aattgactggagggacatcg

SEQ ID NO: 71 - DNA, 20 bp, Artificial Sequence
  ErbB3 right primer
ggagcacagatggtcttggt

SEQ ID NO: 72 - DNA, 20 bp, Artificial Sequence
  ErbB3 hybridization oligonucleotide
aggacaatggcagaagctgt

SEQ ID NO: 73 - DNA, 20 bp, Artificial Sequence
  ErbB3 left primer
aattgactggagggacatcg

SEQ ID NO: 74 - DNA, 20 bp, Artificial Sequence
  ErbB3 right primer
aggagcacagatggtcttgg

SEQ ID NO: 75 - DNA, 20 bp, Artificial Sequence
  ErbB3 hybridization oligonucleotide
aggacaatggcagaagctgt

SEQ ID NO: 76 - DNA, 20 bp, Artificial Sequence
  ErbB3 left primer
aggacaatggcagaagctgt

SEQ ID NO: 77 - DNA, 20 bp, Artificial Sequence
  ErbB3 right primer
cgaggtacacaggctccact

SEQ ID NO: 78 - DNA, 20 bp, Artificial Sequence
  ErbB3 hybridization oligonucleotide
accaagaccatctgtgctcc

SEQ ID NO: 79 - DNA, 20 bp, Artificial Sequence
  ErbB3 left primer
ggaagtttgccatcttcgtc

SEQ ID NO: 80 - DNA, 20 bp, Artificial Sequence
  ErbB3 right primer
acagcttctgccattgtcct

SEQ ID NO: 81 - DNA, 20 bp, Artificial Sequence
  ErbB3 hybridization oligonucleotide
aattgactggagggacatcg

SEQ ID NO: 82 - DNA, 20 bp, Artificial Sequence
  ErbB3 left primer
gagggacccaggtctacgat

SEQ ID NO: 83 - DNA, 20 bp, Artificial Sequence
  ErbB3 right primer
acagcttctgccattgtcct

SEQ ID NO: 84 - DNA, 20 bp, Artificial Sequence
  ErbB3 hybridization oligonucleotide
aattgactggagggacatcg

SEQ ID NO: 85 - DNA, 20 bp, Artificial Sequence
  ErbB4 left primer
tttcgggagtttgagaatgg

SEQ ID NO: 86 - DNA, 20 bp, Artificial Sequence
  ErbB4 right primer
gaaactgtttgccccctgta

SEQ ID NO: 87 - DNA, 20 bp, Artificial Sequence
  ErbB4 hybridization oligonucleotide
aagatggaagatggcctcct

SEQ ID NO: 88 - DNA, 20 bp, Artificial Sequence
  ErbB4 left primer
tttcgggagtttgagaatgg

SEQ ID NO: 89 - DNA, 20 bp, Artificial Sequence
  ErbB4 right primer
gaaactgtttgccccctgta

SEQ ID NO: 90 - DNA, 20 bp, Artificial Sequence
  ErbB4 hybridization oligonucleotide
aagatggaagatggcctcct

SEQ ID NO: 91 - DNA, 20 bp, Artificial Sequence
  ErbB4 left primer
ggtgaatttcgggagtttga

SEQ ID NO: 92 - DNA, 20 bp, Artificial Sequence
  ErbB4 right primer
gaaactgtttgccccctgta

SEQ ID NO: 93 - DNA, 20 bp, Artificial Sequence
  ErbB4 hybridization oligonucleotide
aagatggaagatggcctcct

SEQ ID NO: 94 - DNA, 20 bp, Artificial Sequence
  ErbB4 left primer
ggtgcttttggaacggttta

SEQ ID NO: 95 - DNA, 17 bp, Artificial Sequence
  ErbB4 right primer
aaccggacgtgtggatg

SEQ ID NO: 96 - DNA, 20 bp, Artificial Sequence
  ErbB4 hybridization oligonucleotide
caaggcaaatgtggagttca

SEQ ID NO: 97 - DNA, 20 bp, Artificial Sequence
  ErbB4 left primer
ggtgcttttggaacggttta

SEQ ID NO: 98 - DNA, 17 bp, Artificial Sequence
  ErbB4 right primer
caaccggacgtgtggat

SEQ ID NO: 99 - DNA, 20 bp, Artificial Sequence
  ErbB4 hybridization oligonucleotide
caaggcaaatgtggagttca

SEQ ID NO: 100 - DNA, 20 bp, Artificial Sequence
  ErbB4 left primer
ccagaccaatgtctgtcgtg

SEQ ID NO: 101 - DNA, 20 bp, Artificial Sequence
  ErbB4 right primer
aggaggccatcttccatctt

SEQ ID NO: 102 - DNA, 20 bp, Artificial Sequence
  ErbB4 hybridization oligonucleotide
tttcgggagtttgagaatgg

SEQ ID NO: 103 - DNA, 51 bp, Artificial Sequence
  H1-Forward primer for CDR1
gaggaggaggaggaggaggcggggcccaggcggcccaggtgcagctggtgc

SEQ ID NO: 104 - DNA, 57 bp, Artificial Sequence
  H1-Reverse primer for CDR1
gcggacccagctcatttcataakmakmgaaakmgaaakmagaggctgcacaggagag

SEQ ID NO: 105 - DNA, 45 bp, Artificial Sequence
  H2-Forward1 primer for CDR2
gaaatgagctgggtccgccaggctccaggacaasgscttgagtgg

SEQ ID NO: 106 - DNA, 45 bp, Artificial Sequence
  H2-Forward2 primer for CDR2
gaaatgagctgggtccgccaggctccagggaaggccctggagtgg

SEQ ID NO: 107 - DNA, 45 bp, Artificial Sequence
  H2-Forward3 primer for CDR2
  misc_feature 36: n = A,T,C or G
gaaatgagctgggtccgccaggctccagggaagggnctrgagtgg

SEQ ID NO: 108 - DNA, 36 bp, Artificial Sequence
  H2-Reverse1 primer for CDR2
attgtctctggagatggtgaccctkycctgraacty

SEQ ID NO: 109 - DNA, 36 bp, Artificial Sequence
  H2-Reverse2 primer for CDR2
  misc_feature 34: n = A,T,C or G
attgtctctggagatggtgaatcggcccttcacnga

SEQ ID NO: 110 - DNA, 36 bp, Artificial Sequence
  H2-Reverse3 primer for CDR2
attgtctctggagatggtgactmgactcttgaggga

SEQ ID NO: 111 - DNA, 36 bp, Artificial Sequence
  H2-Reverse4 primer for CDR2
attgtctctggagatggtgacstggccttggaagga

SEQ ID NO: 112 - DNA, 36 bp, Artificial Sequence
  H2-Reverse5 primer for CDR2
attgtctctggagatggtaaaccgtcctgtgaagcc

SEQ ID NO: 113 - DNA, 36 bp, Artificial Sequence
  H3-Forward1 primer for CDR3
accctgagagccgaggacacrgcyttrtattactgt

SEQ ID NO: 114 - DNA, 33 bp, Artificial Sequence
  H3-Forward2 primer for CDR3
accctgagagccgaggacacagccayrtattac

SEQ ID NO: 115 - DNA, 36 bp, Artificial Sequence
  H3-Forward3 primer for CDR3
accctgagagccgaggacacrgcygtrtattactgt

SEQ ID NO: 116 - DNA, 34 bp, Artificial Sequence
  H3-Reverse primer for CDR3
gtggccggcctggccacttgaggagacggtgacc

SEQ ID NO: 117 - DNA, 50 bp, Artificial Sequence
  CDR-H1-Forward primer for CDR
ctctggattcaccttcrrttatkmtatgagctgggtccgccaggctccag

SEQ ID NO: 118 - DNA, 88 bp, Artificial Sequence
  CDR-H2-Forward primer for CDR
gggctggagtgggtctcakbgatctmtymtrrtrrtrgtartahatattacgctgattctgtaaaaggtcggttcaccatctccagag

SEQ ID NO: 119 - DNA, 51 bp, Artificial Sequence
  CDR-H3-9-Reverse primer for CDR
  misc_feature 20, 21, 23, 24, 26, 27, 29, 30: n = A,T,C or G
ctggccccagtagtcgaamnnmnnmnnmnntytcgcacagtaatacacggc

SEQ ID NO: 120 - DNA, 66 bp, Artificial Sequence
  CDR-H3-14-Reverse primer for CDR
  misc_feature 20, 21, 23, 24, 26, 27, 29, 30, 32, 33, 35, 36, 38, 39: n = A,T,C or G
ctggccccagtagtcgaamnnmnnmnnmnnmnnmnnmnnavsayctytcgcacagtaatacacggc

SEQ ID NO: 121 - DNA, 84 bp, Artificial Sequence
  CDR-H3-20-Reverse primer for CDR
  misc_feature 35, 36, 38, 39, 41, 42, 44, 45, 47, 48, 50, 51, 53, 54, 62: n = A,T,C or G
ctggccccagacgtccatascathakmakaakamnnmnnmnnmnnmnnmnnmnnambavbanvtytcgcacagtaatacacggc

SEQ ID NO: 122 - DNA, 84 bp, Artificial Sequence
  CDR-H3-20SS-Reverse primer for CDR
  misc_feature 38, 39, 41, 42, 44, 45, 47, 48, 53, 54, 62: n = A,T,C or G
ctggccccagacgtccatascathakmakaakaacamnnmnnmnnmnnacamnnambavbanctytcgcacagtaatacacggc

SEQ ID NO: 123 - DNA, 77 bp, Artificial Sequence
  CDR-L1-Forward primer for CDR
gagggtcaccatctcttgtastggctcttcatctaatattggcartaatdmtgtcwmctggtaccagcagctcccag

SEQ ID NO: 124 - DNA, 57 bp, Artificial Sequence
  CDR-L2-Forward primer for CDR
cccaaactcctcatctatkmtratartmakcggccaagcggggtccctgaccgattc

SEQ ID NO: 125 - DNA, 63 bp, Artificial Sequence
  CDR-L3-Reverse primer for CDR
gaggctgattattactgtgstdcttgggatkmtagcctgartgsttatgtcttcggcggaggc

SEQ ID NO: 126 - DNA, 21 bp, Artificial Sequence
  FR3-Forward primer for FR
accatctccagagacaattcc

SEQ ID NO: 127 - DNA, 19 bp, Artificial Sequence
  FR3-Reverse primer for FR
gtcctcggctctcagggtg

SEQ ID NO: 128 - DNA, 54 bp, Artificial Sequence
  FR-H1-Forward primer for FR
gaggtgcagctgttggagtctgggggaggcttggtacagcctggggggtccctg

SEQ ID NO: 129 - DNA, 54 bp, Artificial Sequence
  FR-H1-Reverse primer for FR
gctaaaggtgaatccagaggctgcacaggagagtctcagggaccccccaggctg

SEQ ID NO: 130 - DNA, 42 bp, Artificial Sequence
  FR-H2-Reverse primer for FR
tgagacccactccagccccttccctggagcctggcggaccca

SEQ ID NO: 131 - DNA, 58 bp, Artificial Sequence
  FR-H3-Reverse primer for FR
ggctgttcatttgcagatacagcgtgttcttggaattgtctctggagatggtgaaccg

SEQ ID NO: 132 - DNA, 55 bp, Artificial Sequence
  FR-H3-Forward primer for FR
gtatctgcaaatgaacagcctgagagccgaggacacggccgtgtattactgtgcg

SEQ ID NO: 133 - DNA, 42 bp, Artificial Sequence
  JH-15-Forward primer for FR
ttcgactactggggccagggtacactggtcaccgtgagctca

SEQ ID NO: 134 - DNA, 42 bp, Artificial Sequence
  JH-6-Forward primer for FR
atggactgctggggccagggtacactggtcaccgtgagctca

SEQ ID NO: 135 - DNA, 42 bp, Artificial Sequence
  FR-L1-Forward primer for FR
cagtctgtgctgactcagccaccctcagcgtctgggaccccc

SEQ ID NO: 136 - DNA, 43 bp, Artificial Sequence
  FR-L1-Reverse primer for FR
acaagagatggtgaccctctgcccgggggtcccagacgctgag

SEQ ID NO: 137 - DNA, 46 bp, Artificial Sequence
  FR-L2-Reverse primer for FR
atagatgaggagtttgggggccgttcctgggagctgctggtaccag

SEQ ID NO: 138 - DNA, 57 bp, Artificial Sequence
  FR-L3-Reverse primer for FR
gatggccagggaggctgaggtgccagacttggagccagagaatcggtcagggacccc

SEQ ID NO: 139 - DNA, 58 bp, Artificial Sequence
  FR-L3-F primer for FR
tcagcctccctggccatcagtgggctccggtccgaggatgaggctgattattactgtg

SEQ ID NO: 140 - DNA, 39 bp, Artificial Sequence
  JL-Forward primer for FR
tatgtcttcggcggaggcaccaagctgacggtcctaggc

SEQ ID NO: 141 - DNA, 19 bp, Artificial Sequence
  FRH3-short-Reverse primer for FR
cgcacagtaatacacggcc

SEQ ID NO: 142 - DNA, 18 bp, Artificial Sequence
  JH15-short-Forward primer for FR
ttcgactactggggccag

SEQ ID NO: 143 - DNA, 27 bp, Artificial Sequence
  JH6-short-Forward primer for FR
atggacgtctggggccagggtacactg

SEQ ID NO: 144 - DNA, 19 bp, Artificial Sequence
  pC3X-Forward primer for FR
gcacgacaggtttcccgac

SEQ ID NO: 145 - DNA, 17 bp, Artificial Sequence
  pC3X-Reverse primer for FR
aaccatcgacagcaccg

SEQ ID NO: 146 - DNA, 19 bp, Artificial Sequence
  H1-Reverse primer for FR
gctaaaggtgaatccagag

SEQ ID NO: 147 - DNA, 20 bp, Artificial Sequence
  H2-Forward primer for FR
ctgggtccgccaggctccag

SEQ ID NO: 148 - DNA, 18 bp, Artificial Sequence
  H2-Reverse primer for FR
tgagacccactccagccc

SEQ ID NO: 149 - DNA, 19 bp, Artificial Sequence
  H3-Forward primer for FR
cggttcaccatctccagag

SEQ ID NO: 150 - DNA, 18 bp, Artificial Sequence
  L1-Reverse primer for FR
caagagatggtgaccctc

SEQ ID NO: 151 - DNA, 20 bp, Artificial Sequence
  L2-Forward primer for FR
ctggtaccagcagctcccag

SEQ ID NO: 152 - DNA, 18 bp, Artificial Sequence
  L2-Reverse primer for FR
atagatgaggagtttggg

SEQ ID NO: 153 - DNA, 18 bp, Artificial Sequence
  L3-Forward primer for FR
ggggtccctgaccgattc

SEQ ID NO: 154 - DNA, 19 bp, Artificial Sequence
  L3-Reverse primer for FR
cacagtaataatcagcctc

SEQ ID NO: 155 - DNA, 18 bp, Artificial Sequence
  JL-short-Forward primer for FR
tatgtcttcggcggaggc

SEQ ID NO: 156 - DNA, 33 bp, Artificial Sequence
  anti-huEGFR H1a consensus sequence
  misc_feature 3, 9, 15, 18, 24: n = A,T,C or G
ggnttywsnttywsnacntayggnatgcaytrr

SEQ ID NO: 157 - DNA, 48 bp, Artificial Sequence
  anti-huEGFR H2a consensus sequence
  misc_feature 3, 18, 21, 36, 42, 45: n = A,T,C or G
gtnathtgggaygayggnwsntayaartayttyggngaywsngtntrr

SEQ ID NO: 158 - DNA, 48 bp, Artificial Sequence; identical to SEQ ID NO: 157
  anti-huEGFR H3a consensus sequence
  misc_feature 3, 18, 21, 36, 42, 45: n = A,T,C or G

SEQ ID NO: 159 - DNA, 33 bp, Artificial Sequence
  anti-huEGFR H1b consensus sequence
  misc_feature 3, 9, 15, 18, 24: n = A,T,C or G
ggnttyacntaywsnacntayggnatgcaytrr

SEQ ID NO: 160 - DNA, 48 bp, Artificial Sequence
  anti-huEGFR H2b consensus sequence
  misc_feature 3, 18, 21, 36, 42, 45: n = A,T,C or G
gtnathtgggargayggnwsntayaartaytayggngaywsngtntrr

SEQ ID NO: 161 - DNA, 51 bp, Artificial Sequence
  anti-huEGFR H3b consensus sequence
  misc_feature 6, 12, 18, 21, 24, 27, 33: n = A,T,C or G
gayggnathwsnatggtnmgngcngtnatgmgngaytayttygayttytrr

SEQ ID NO: 162 - DNA, 33 bp, Artificial Sequence
  anti-huEGFR H1c consensus sequence
  misc_feature 3, 9, 15, 18, 24: n = A,T,C or G
ggnttyacnttywsnacnttygcnatgcaytrr

SEQ ID NO: 163 - DNA, 48 bp, Artificial Sequence
  anti-huEGFR H2c consensus sequence
  misc_feature 3, 18, 21, 36, 42, 45: n = A,T,C or G
gtnathtgggaygayggnwsntayaarttytaygcngarwsngtntrr

SEQ ID NO: 164 - DNA, 51 bp, Artificial Sequence
  anti-huEGFR H3c consensus sequence
  misc_feature 6, 12, 18, 21, 24, 27, 33: n = A,T,C or G
gayggnathacnatggtnmgnggngtnatgmgngaytayttygayttytrr

SEQ ID NO: 165 - DNA, 36 bp, Artificial Sequence
  anti-huEGFR L1a consensus sequence
  misc_feature 3, 6, 9, 21, 24, 27, 30, 33: n = A,T,C or G
mgngcnwsncargayathwsnwsngcnytngtntrr

SEQ ID NO: 166 - DNA, 21 bp, Artificial Sequence
  anti-huEGFR L2a consensus sequence
  misc_feature 6, 9, 12, 15: n = A,T,C or G
gaygcnwsnwsnytngartrr

SEQ ID NO: 167 - DNA, 30 bp, Artificial Sequence
  anti-huEGFR L3a consensus sequence
  misc_feature 15, 21, 24, 27: n = A,T,C or G
carcarttyaaywsntayccnytnacntrr

SEQ ID NO: 168 - DNA, 36 bp, Artificial Sequence
  anti-huEGFR L1b consensus sequence
  misc_feature 3, 6, 9, 21, 24, 27, 30, 33: n = A,T,C or G
mgngcnwsncargarathwsnwsngcnytnytntrr

SEQ ID NO: 169 - DNA, 24 bp, Artificial Sequence
  anti-huEGFR L2b consensus sequence
  misc_feature 6, 9, 12, 15, 21: n = A,T,C or G
gargcnwsnwsnytngaracntrr

SEQ ID NO: 170 - DNA, 30 bp, Artificial Sequence
  anti-huEGFR L3b consensus sequence
  misc_feature 15, 21, 24, 27: n = A,T,C or G
caraayttyaaywsntayccnytnwsntrr

SEQ ID NO: 171 - DNA, 36 bp, Artificial Sequence
  anti-huEGFR L1c consensus sequence
  misc_feature 3, 6, 9, 21, 24, 27, 30, 33: n = A,T,C or G
mgngcnwsncargayathacnwsngcnytnytntrr

SEQ ID NO: 172 - DNA, 21 bp, Artificial Sequence
  anti-huEGFR L2c consensus sequence
  misc_feature 6, 9, 12, 15, 21: n = A,T,C or G
gaygcnwsnwsnytngarwsn

SEQ ID NO: 173 - DNA, 27 bp, Artificial Sequence
  anti-huEGFR L3c consensus sequence
  misc_feature 15, 21, 24, 27: n = A,T,C or G
aaycarttycarwsntayccnytnwsn

SEQ ID NO: 174 - DNA, 33 bp, Artificial Sequence
  anti-huEGFRvIII H1a consensus sequence
  misc_feature 3, 9, 15, 24, 30: n = A,T,C or G
ggnttywsnttymgnaarttyggnatgwsntrr

SEQ ID NO: 175 - DNA, 48 bp, Artificial Sequence
  anti-huEGFRvIII H2a consensus sequence
  misc_feature 3, 9, 12, 15, 18, 27, 36, 45: n = A,T,C or G
wsnathwsnacnggnggntayaaywsntaytaywsngayaaygtntrr

SEQ ID NO: 176 - DNA, 36 bp, Artificial Sequence
  anti-huEGFRvIII H3a consensus sequence
  misc_feature 3, 9, 12, 15, 18, 24: n = A,T,C or G
ggnttywsnwsnacnwsntaygcnatggaytaytrr

SEQ ID NO: 177 - DNA, 33 bp, Artificial Sequence
  anti-huEGFRvIII H1b consensus sequence
  misc_feature 3, 9, 24, 30: n = A,T,C or G
ggnttyacnttyaaraarttyggnatgwsntrr

SEQ ID NO: 178 - DNA, 48 bp, Artificial Sequence
  anti-huEGFRvIII H2b consensus sequence
  misc_feature 3, 9, 12, 15, 18, 27, 36, 45: n = A,T,C or G
wsnathwsnacnggnggnttyaayacntaytaywsngayaaygtntrr

SEQ ID NO: 179 - DNA, 36 bp, Artificial Sequence
  anti-huEGFRvIII H3b consensus sequence
  misc_feature 3, 9, 12, 15, 18, 24: n = A,T,C or G
ggntaywsnwsnacnwsnttyggnatggaytaytrr

SEQ ID NO: 180 - DNA, 33 bp, Artificial Sequence
  anti-huEGFRvIII H1c consensus sequence
  misc_feature 3, 9, 15, 24, 30: n = A,T,C or G
ggntaywsnttymgnaarttyggnatgwsntrr

SEQ ID NO: 181 - DNA, 48 bp, Artificial Sequence
  anti-huEGFRvIII H2c consensus sequence
  misc_feature 3, 9, 12, 15, 18, 27, 36, 45: n = A,T,C or G
wsnathwsnacnggnggntaycaracntaytaywsngayaaygtntrr

SEQ ID NO: 182 - DNA, 36 bp, Artificial Sequence
  anti-huEGFRvIII H3c consensus sequence
  misc_feature 3, 9, 12, 15, 18, 24: n = A,T,C or G
ggntaywsnwsnacnwsntaygcnatggayttytrr

SEQ ID NO: 183 - DNA, 48 bp, Artificial Sequence
  anti-huEGFRvIII L1a consensus sequence
  misc_feature 3, 6, 9, 15, 18, 24, 30, 36: n = A,T,C or G
mgngcnwsncarwsngtncaywsngayggnaayacntayatgcartrr

SEQ ID NO: 184 - DNA, 24 bp, Artificial Sequence
  anti-huEGFRvIII L2a consensus sequence
  misc_feature 3, 6, 9, 15, 21: n = A,T,C or G
gcngcnwsnaaymgnttywsntrr

SEQ ID NO: 185 - DNA, 30 bp, Artificial Sequence
  anti-huEGFRvIII L3a consensus sequence
  misc_feature 9, 12, 18, 21, 24, 27: n = A,T,C or G
carcarggnacncarytnccnmgnacntrr

SEQ ID NO: 186 - DNA, 48 bp, Artificial Sequence
  anti-huEGFRvIII L1b consensus sequence
  misc_feature 3, 6, 9, 15, 18, 24, 30, 36, 42, 45: n = A,T,C or G
mgnwsnwsncarwsngtncaywsngayggnaaywsntayytnwsntrr

SEQ ID NO: 187 - DNA, 24 bp, Artificial Sequence
  anti-huEGFRvIII L2b consensus sequence
  misc_feature 3, 6, 9, 21: n = A,T,C or G
ggngcnwsnaayaarttywsntrr

SEQ ID NO: 188 - DNA, 30 bp, Artificial Sequence; identical to SEQ ID NO: 185
  anti-huEGFRvIII L3b consensus sequence
  misc_feature 9, 12, 18, 21, 24, 27: n = A,T,C or G

SEQ ID NO: 189 - DNA, 48 bp, Artificial Sequence
  anti-huEGFRvIII L1c consensus sequence
  misc_feature 6, 12, 15, 18, 24, 30, 36, 42, 45: n = A,T,C or G
aarwsncarwsnytngtncaywsngayggnaaywsntayytnwsntrr

SEQ ID NO: 190 - DNA, 24 bp, Artificial Sequence
  anti-huEGFRvIII L2c consensus sequence
  misc_feature 3, 9, 15, 21: n = A,T,C or G
mgnathwsnaaymgnttywsntrr

SEQ ID NO: 191 - DNA, 30 bp, Artificial Sequence; identical to SEQ ID NO: 185
  anti-huEGFRvIII L3c consensus sequence
  misc_feature 9, 12, 18, 21, 24, 27: n = A,T,C or G

SEQ ID NO: 192 - DNA, 30 bp, Artificial Sequence
  anti-huTfR H1a consensus sequence
  misc_feature 3, 9, 15, 18: n = A,T,C or G
ggntaywsntaywsnwsntaytggatgtrr

SEQ ID NO: 193 - DNA, 48 bp, Artificial Sequence
  anti-huTfR H2a consensus sequence
  misc_feature 3, 12, 15, 21, 27, 39: n = A,T,C or G
gcnathgayccnmgnaaywsngayacnathtayaayccncarttytrr

SEQ ID NO: 194 - DNA, 21 bp, Artificial Sequence
  anti-huTfR H3a consensus sequence
  misc_feature 3, 18: n = A,T,C or G
ytntaytaytaygaywsntrr

SEQ ID NO: 195 - DNA, 30 bp, Artificial Sequence
  anti-huTfR H1b consensus sequence
  misc_feature 3, 9, 15, 18: n = A,T,C or G
ggntayacnathwsnwsntaytggatgtrr

SEQ ID NO: 196 - DNA, 48 bp, Artificial Sequence
  anti-huTfR H2b consensus sequence
  misc_feature 3, 6, 12, 15, 21, 27, 39: n = A,T,C or G
gcngcngayccnmgnaaywsngayacnathtaycarccncartaytrr

SEQ ID NO: 197 - DNA, 21 bp, Artificial Sequence
  anti-huTfR H3b consensus sequence
  misc_feature 3, 18: n = A,T,C or G
ytntaytayttygaywsntrr

SEQ ID NO: 198 - DNA, 30 bp, Artificial Sequence
  anti-huTfR H1c consensus sequence
  misc_feature 3, 9, 12, 15, 18: n = A,T,C or G
ggntayacngcnacnacntaytggatgtrr

SEQ ID NO: 199 - DNA, 42 bp, Artificial Sequence
  anti-huTfR H2c consensus sequence
  misc_feature 12, 15, 21, 27, 30, 33: n = A,T,C or G
atgathcayccnwsngaywsngargtnmgnytnaaycartrr

SEQ ID NO: 200 - DNA, 21 bp, Artificial Sequence
  anti-huTfR H3c consensus sequence
  misc_feature 3, 18: n = A,T,C or G
ytntaytayttygarwsntrr

SEQ ID NO: 201 - DNA, 24 bp, Artificial Sequence
  anti-huTfR L1a consensus sequence
  misc_feature 18: n = A,T,C or G
gayathaayaaytaygtntgytrr

SEQ ID NO: 202 - DNA, 24 bp, Artificial Sequence
  anti-huTfR L2a consensus sequence
  misc_feature 6, 12, 15, 18: n = A,T,C or G
aargcnaaymgnytngtngaytrr

SEQ ID NO: 203 - DNA, 30 bp, Artificial Sequence
  anti-huTfR L3a consensus sequence
  misc_feature 3, 21, 27: n = A,T,C or G
ytncartaygaygarttyccntayacntrr

SEQ ID NO: 204 - DNA, 24 bp, Artificial Sequence
  anti-huTfR L1b consensus sequence
  misc_feature 18: n = A,T,C or G
garathaayaaytayytntgytrr

SEQ ID NO: 205 - DNA, 24 bp, Artificial Sequence
  anti-huTfR L2b consensus sequence
  misc_feature 3, 6, 15, 18: n = A,T,C or G
mgngcnaayaarytngtngaytrr

SEQ ID NO: 206 - DNA, 30 bp, Artificial Sequence
  anti-huTfR L3b consensus sequence
  misc_feature 3, 21, 27: n = A,T,C or G
ytncartaygaygayttyccntayacntrr

SEQ ID NO: 207 - DNA, 24 bp, Artificial Sequence
  anti-huTfR L1c consensus sequence
  misc_feature 18: n = A,T,C or G
gayathaaycarttyytntgytrr

SEQ ID NO: 208 - DNA, 24 bp, Artificial Sequence
  anti-huTfR L2c consensus sequence
  misc_feature 3, 6, 12, 15, 18: n = A,T,C or G
mgngcnaaymgnytngtngaytrr

SEQ ID NO: 209 - DNA, 30 bp, Artificial Sequence
  anti-huTfR L3c consensus sequence
  misc_feature 3, 21, 27: n = A,T,C or G
gtncartaygaygarttyccntaywsntrr

SEQ ID NO: 210 - DNA, 27 bp, Artificial Sequence
  anti-huEGFR H1a exemplar sequence
ggcttcttcacctatggcatgcattaa

SEQ ID NO: 211 - DNA, 45 bp, Artificial Sequence
  anti-huEGFR H2a exemplar sequence
gtgatttgggatgatggcagctataaatattttggcgacgtgtaa

SEQ ID NO: 212 - DNA, 45 bp, Artificial Sequence; identical to SEQ ID NO: 211
  anti-huEGFR H3a exemplar sequence

SEQ ID NO: 213 - DNA, 30 bp, Artificial Sequence
  anti-huEGFR H1b exemplar sequence
ggctttacctacacctatggcatgcattaa

SEQ ID NO: 214 - DNA, 45 bp, Artificial Sequence
  anti-huEGFR H2b exemplar sequence
gtgatttgggaagatggcagctataaatattatggcgacgtgtaa

SEQ ID NO: 215 - DNA, 48 bp, Artificial Sequence
  anti-huEGFR H3b exemplar sequence
gatggcatcatggtgcgcgcggtgatgcgcgattattttgatttttaa

SEQ ID NO: 216 - DNA, 30 bp, Artificial Sequence
  anti-huEGFR H1c exemplar sequence
ggctttaccttcacctttgcgatgcattaa

SEQ ID NO: 217 - DNA, 48 bp, Artificial Sequence
  anti-huEGFR H2c exemplar sequence
gtgatttgggatgatggcagctataaattttatgcggaaagcgtgtaa

SEQ ID NO: 218 - DNA, 51 bp, Artificial Sequence
  anti-huEGFR H3c exemplar sequence
gatggcattaccatggtgcgcggcgtgatgcgcgattattttgatttttaa

SEQ ID NO: 219 - DNA, 33 bp, Artificial Sequence
  anti-huEGFR L1a exemplar sequence
cgcgcgagccaggatatcagcgcgctggtgtaa

SEQ ID NO: 220 - DNA, 21 bp, Artificial Sequence
  anti-huEGFR L2a exemplar sequence
gatgcgagcagcctggaataa

SEQ ID NO: 221 - DNA, 30 bp, Artificial Sequence
  anti-huEGFR L3a exemplar sequence
cagcagtttaacagctatccgctgacctaa

SEQ ID NO: 222 - DNA, 33 bp, Artificial Sequence
  anti-huEGFR L1b exemplar sequence
cgcgcgagccaggaaatcagcgcgctgctgtaa

SEQ ID NO: 223 - DNA, 24 bp, Artificial Sequence
  anti-huEGFR L2b exemplar sequence
gaagcgagcagcctggaaacctaa

SEQ ID NO: 224 - DNA, 30 bp, Artificial Sequence
  anti-huEGFR L3b exemplar sequence
cagaactttaacagctatccgctgagctaa

SEQ ID NO: 225 - DNA, 36 bp, Artificial Sequence
  anti-huEGFR L1c exemplar sequence
cgcgcgagccaggatattaccagcgcgctgctgtaa

SEQ ID NO: 226 - DNA, 21 bp, Artificial Sequence
  anti-huEGFR L2c exemplar sequence
gatgcgagcagcctggaaagc

SEQ ID NO: 227 - DNA, 27 bp, Artificial Sequence
  anti-huEGFR L3c exemplar sequence
aaccagtttcagagctatccgctgagc

SEQ ID NO: 228 - DNA, 30 bp, Artificial Sequence
  anti-huEGFRvIII H1a exemplar sequence
ggcttctttcgcaaatttggcatgagctaa

SEQ ID NO: 229 - DNA, 42 bp, Artificial Sequence
  anti-huEGFRvIII H2a exemplar sequence
agcatcaccggcggctataacagctattacgataacgtgtaa

SEQ ID NO: 230 - DNA, 33 bp, Artificial Sequence
  anti-huEGFRvIII H3a exemplar sequence
ggcttcagcaccagctatgcgatggattattaa

SEQ ID NO: 231 - DNA, 33 bp, Artificial Sequence
  anti-huEGFRvIII H1b exemplar sequence
ggctttacctttaaaaaatttggcatgagctaa

SEQ ID NO: 232 - DNA, 42 bp, Artificial Sequence
  anti-huEGFRvIII H2b exemplar sequence
agcatcaccggcggctttaacacctattacgataacgtgtaa

SEQ ID NO: 233 - DNA, 33 bp, Artificial Sequence
  anti-huEGFRvIII H3b exemplar sequence
ggctacagcaccagctttggcatggattattaa

SEQ ID NO: 234 - DNA, 30 bp, Artificial Sequence
  anti-huEGFRvIII H1c exemplar sequence
ggctactttcgcaaatttggcatgagctaa

SEQ ID NO: 235 - DNA, 42 bp, Artificial Sequence
  anti-huEGFRvIII H2c exemplar sequence
agcatcaccggcggctatcagacctattacgataacgtgtaa

SEQ ID NO: 236 - DNA, 33 bp, Artificial Sequence
  anti-huEGFRvIII H3c exemplar sequence
ggctacagcaccagctatgcgatggatttttaa

SEQ ID NO: 237 - DNA, 45 bp, Artificial Sequence
  anti-huEGFRvIII L1a exemplar sequence
cgcgcgagccagagcgtgcacgatggcaacacctatatgcagtaa

SEQ ID NO: 238 - DNA, 21 bp, Artificial Sequence
  anti-huEGFRvIII L2a exemplar sequence
gcggcgagcaaccgcttctaa

SEQ ID NO: 239 - DNA, 30 bp, Artificial Sequence
  anti-huEGFRvIII L3a exemplar sequence
cagcagggcacccagctgccgcgcacctaa

SEQ ID NO: 240 - DNA, 45 bp, Artificial Sequence
  anti-huEGFRvIII L1b exemplar sequence
cgcagcagccagagcgtgcacgatggcaacagctatctgagctaa

SEQ ID NO: 241 - DNA, 21 bp, Artificial Sequence
  anti-huEGFRvIII L2b exemplar sequence
ggcgcgagcaacaaattctaa

SEQ ID NO: 242 - DNA, 30 bp, Artificial Sequence; identical to SEQ ID NO: 239
  anti-huEGFRvIII L3b exemplar sequence

SEQ ID NO: 243 - DNA, 45 bp, Artificial Sequence
  anti-huEGFRvIII L1c exemplar sequence
aaaagccagagcctggtgcacgatggcaacagctatctgagctaa

SEQ ID NO: 244 - DNA, 18 bp, Artificial Sequence
  anti-huEGFRvIII L2c exemplar sequence
cgcatcaaccgcttctaa

SEQ ID NO: 245 - DNA, 30 bp, Artificial Sequence; identical to SEQ ID NO: 239
  anti-huEGFRvIII L3c exemplar sequence

SEQ ID NO: 246 - DNA, 24 bp, Artificial Sequence
  anti-huTfR H1a exemplar sequence
ggctactacagctattggatgtaa

SEQ ID NO: 247 - DNA, 48 bp, Artificial Sequence
  anti-huTfR H2a exemplar sequence
gcgattgatccgcgcaacagcgataccatttataacccgcagttttaa

SEQ ID NO: 248 - DNA, 18 bp, Artificial Sequence
  anti-huTfR H3a exemplar sequence
ctgtattattatgactaa

SEQ ID NO: 249 - DNA, 27 bp, Artificial Sequence
  anti-huTfR H1b exemplar sequence
ggctataccatcagctattggatgtaa

SEQ ID NO: 250 - DNA, 48 bp, Artificial Sequence
  anti-huTfR H2b exemplar sequence
gcggcggatccgcgcaacagcgataccatttatcagccgcagtattaa

SEQ ID NO: 251 - DNA, 18 bp, Artificial Sequence
  anti-huTfR H3b exemplar sequence
ctgtattattttgactaa

SEQ ID NO: 252 - DNA, 30 bp, Artificial Sequence
  anti-huTfR H1c exemplar sequence
ggctataccgcgaccacctattggatgtaa

SEQ ID NO: 253 - DNA, 39 bp, Artificial Sequence
  anti-huTfR H2c exemplar sequence
atgattcatccgagcgacgaagtgcgcctgaaccagtaa

SEQ ID NO: 254 - DNA, 21 bp, Artificial Sequence
  anti-huTfR H3c exemplar sequence
ctgtattattttgaaagctaa

SEQ ID NO: 255 - DNA, 24 bp, Artificial Sequence
  anti-huTfR L1a exemplar sequence
gatattaacaactatgtgtgctaa

SEQ ID NO: 256 - DNA, 24 bp, Artificial Sequence
  anti-huTfR L2a exemplar sequence
aaagcgaaccgcctggtggattaa

SEQ ID NO: 257 - DNA, 30 bp, Artificial Sequence
  anti-huTfR L3a exemplar sequence
ctgcagtatgatgaatttccgtatacctaa

SEQ ID NO: 258 - DNA, 24 bp, Artificial Sequence
  anti-huTfR L1b exemplar sequence
gaaattaacaactatctgtgctaa

SEQ ID NO: 259 - DNA, 24 bp, Artificial Sequence
  anti-huTfR L2b exemplar sequence
cgcgcgaacaaactggtggattaa

SEQ ID NO: 260 - DNA, 30 bp, Artificial Sequence
  anti-huTfR L3b exemplar sequence
ctgcagtatgatgattttccgtatacctaa

SEQ ID NO: 261 - DNA, 24 bp, Artificial Sequence
  anti-huTfR L1c exemplar sequence
gatattaaccagtttctgtgctaa

SEQ ID NO: 262 - DNA, 24 bp, Artificial Sequence
  anti-huTfR L2c exemplar sequence
cgcgcgaaccgcctggtggattaa

SEQ ID NO: 263 - DNA, 27 bp, Artificial Sequence
  anti-huTfR L3c exemplar sequence
gtgcagtatgatgaatttccgtactaa

SEQ ID NO: 264 - protein, 10 aa, Artificial Sequence
  anti-huEGFR H1a translation
GFSFSTYGMH

SEQ ID NO: 265 - protein, 15 aa, Artificial Sequence
  anti-huEGFR H2a translation
VIWDDGSYKYFGDSV

SEQ ID NO: 266 - protein, 16 aa, Artificial Sequence
  anti-huEGFR H3a translation
DAITMVRGVMKEYFDY

SEQ ID NO: 267 - protein, 10 aa, Artificial Sequence
  anti-huEGFR H1b translation
GFTYSTYGMH

SEQ ID NO: 268 - protein, 15 aa, Artificial Sequence
  anti-huEGFR H2b translation
VIWEDGSYKYYGDSV

SEQ ID NO: 269 - protein, 16 aa, Artificial Sequence
  anti-huEGFR H3b translation
DGISMVRAVMRDYFDF

SEQ ID NO: 270 - protein, 10 aa, Artificial Sequence
  anti-huEGFR H1c translation
GFTFSTFAMH

SEQ ID NO: 271 - protein, 15 aa, Artificial Sequence
  anti-huEGFR H2c translation
VIWDDGSYKFYAESV

SEQ ID NO: 272 - protein, 16 aa, Artificial Sequence
  anti-huEGFR H3c translation
DGITMVRGVMRDYFDF

SEQ ID NO: 273 - protein, 11 aa, Artificial Sequence
  anti-huEGFR L1a translation
RASQDISSALV

SEQ ID NO: 274 - protein, 6 aa, Artificial Sequence
  anti-huEGFR L2a translation
DASSLE

SEQ ID NO: 275 - protein, 9 aa, Artificial Sequence
  anti-huEGFR L3a translation
QQFNSYPLT

SEQ ID NO: 276 - protein, 11 aa, Artificial Sequence
  anti-huEGFR L1b translation
RASQEISSALL

SEQ ID NO: 277 - protein, 7 aa, Artificial Sequence
  anti-huEGFR L2b translation
EASSLET

SEQ ID NO: 278 - protein, 9 aa, Artificial Sequence
  anti-huEGFR L3b translation
QNFNSYPLS

SEQ ID NO: 279 - protein, 11 aa, Artificial Sequence
  anti-huEGFR L1c translation
RASQDITSALL

SEQ ID NO: 280 - protein, 7 aa, Artificial Sequence
  anti-huEGFR L2c translation
DASSLES

SEQ ID NO: 281 - protein, 9 aa, Artificial Sequence
  anti-huEGFR L3c translation
NQFQSYPLS

SEQ ID NO: 282 - protein, 10 aa, Artificial Sequence
  anti-huEGFRvIII H1a translation
GFSFRKFGMS

SEQ ID NO: 283 - protein, 15 aa, Artificial Sequence
  anti-huEGFRvIII H2a translation
SISTGGYNSYYSDNV

SEQ ID NO: 284 - protein, 11 aa, Artificial Sequence
  anti-huEGFRvIII H3a translation
GFSSTSYAMDY

SEQ ID NO: 285 - protein, 10 aa, Artificial Sequence
  anti-huEGFRvIII H1b translation
GFTFKKFGMS

SEQ ID NO: 286 - protein, 15 aa, Artificial Sequence
  anti-huEGFRvIII H2b translation
SISTGGFNTYYSDNV

SEQ ID NO: 287 - protein, 11 aa, Artificial Sequence
  anti-huEGFRvIII H3b translation
GYSSTSFGMDY

SEQ ID NO: 288 - protein, 10 aa, Artificial Sequence
  anti-huEGFRvIII H1c translation
GYSFRKFGMS

SEQ ID NO: 289 - protein, 15 aa, Artificial Sequence
  anti-huEGFRvIII H2c translation
SISTGGYQTYYSDNV

SEQ ID NO: 290 - protein, 11 aa, Artificial Sequence
  anti-huEGFRvIII H3c translation
GYSSTSYAMDF

SEQ ID NO: 291 - protein, 15 aa, Artificial Sequence
  anti-huEGFRvIII L1a translation
RASQSVHSDGNTYMQ

SEQ ID NO: 292 - protein, 7 aa, Artificial Sequence
  anti-huEGFRvIII L2a translation
AASNRFS

SEQ ID NO: 293 - protein, 9 aa, Artificial Sequence
  anti-huEGFRvIII L3a translation
QQGTQLPRT

SEQ ID NO: 294 - protein, 15 aa, Artificial Sequence
  anti-huEGFRvIII L1b translation
RSSQSVHSDGNSYLS

SEQ ID NO: 295 - protein, 7 aa, Artificial Sequence
  anti-huEGFRvIII L2b translation
GASNKFS

SEQ ID NO: 296 - protein, 9 aa, Artificial Sequence
  anti-huEGFRvIII L3b translation
QQGTQLPRT

SEQ ID NO: 297 - protein, 15 aa, Artificial Sequence
  anti-huEGFRvIII L1c translation
KSQSLVHSDGNSYLS

SEQ ID NO: 298 - protein, 7 aa, Artificial Sequence
  anti-huEGFRvIII L2c translation
RISNRFS

SEQ ID NO: 299 - protein, 9 aa, Artificial Sequence
  anti-huEGFRvIII L3c translation
QQGTQLPRT

SEQ ID NO: 300 - protein, 9 aa, Artificial Sequence
  anti-huTfR H1a translation
GYSYSSYWM

SEQ ID NO: 301 - protein, 15 aa, Artificial Sequence
  anti-huTfR H2a translation
AIDPRNSDTIYNPQF

SEQ ID NO: 302 - protein, 6 aa, Artificial Sequence
  anti-huTfR H3a translation
LYYYDS

SEQ ID NO: 303 - protein, 9 aa, Artificial Sequence
  anti-huTfR H1b translation
GYTISSYWM

SEQ ID NO: 304 - protein, 15 aa, Artificial Sequence
  anti-huTfR H2b translation
AADPRNSDTIYQPQY

SEQ ID NO: 305 - protein, 6 aa, Artificial Sequence
  anti-huTfR H3b translation
LYYFDS

SEQ ID NO: 306 - protein, 9 aa, Artificial Sequence
  anti-huTfR H1c translation
GYTATTYWM

SEQ ID NO: 307 - protein, 13 aa, Artificial Sequence
  anti-huTfR H2c translation
MIHPSDSEVRLNQ

SEQ ID NO: 308 - protein, 6 aa, Artificial Sequence
  anti-huTfR H3c translation
LYYFES

SEQ ID NO: 309 - protein, 7 aa, Artificial Sequence
  anti-huTfR L1a translation
DINNYVC

SEQ ID NO: 310 - protein, 7 aa, Artificial Sequence
  anti-huTfR L2a translation
KANRLVD

SEQ ID NO: 311 - protein, 9 aa, Artificial Sequence
  anti-huTfR L3a translation
LQYDEFPYT

SEQ ID NO: 312 - protein, 7 aa, Artificial Sequence
  anti-huTfR L1b translation
EINNYLC

SEQ ID NO: 313 - protein, 7 aa, Artificial Sequence
  anti-huTfR L2b translation
RANKLVD

SEQ ID NO: 314 - protein, 9 aa, Artificial Sequence
  anti-huTfR L3b translation
LQYDDFPYT

SEQ ID NO: 315 - protein, 7 aa, Artificial Sequence
  anti-huTfR L1c translation
DINQFLC

SEQ ID NO: 316 - protein, 7 aa, Artificial Sequence
  anti-huTfR L2c translation
RANRLVD

SEQ ID NO: 317 - protein, 9 aa, Artificial Sequence
  anti-huTfR L3c translation
VQYDEFPYS

SEQ ID NO: 318 - protein, 450 aa, Artificial Sequence
  Trastuzumab Humanized H-GAMMA-1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 214 aa, Artificial Sequence
  Trastuzumab Humanized L-KAPPA (V-KAPPA)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 449 aa, Artificial Sequence
  Cetuximab Chimeric H-GAMMA-1
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 214 aa, Artificial Sequence
  Cetuximab Chimeric L-KAPPA (V-KAPPA)
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 448 aa, Artificial Sequence
  Pertuzumab Humanized H
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 214 aa, Artificial Sequence
  Pertuzumab Humanized H
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 451 aa, Artificial Sequence
  Rituximab Chimeric H
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 213 aa, Artificial Sequence
  Rituximab Chimeric L
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 451 aa, Artificial Sequence
  Alemtuzumab Humanized H-GAMMA-1
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 214 aa, Artificial Sequence
  Alemtuzumab Humanized L-KAPPA
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 226 aa, Hepatitis B virus
  ref <308>: DATABASE ACCESSION NUMBER: GenBank J02205.1
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 329 - DNA, 892 bp, Hepatitis B virus
  ref <308>: DATABASE ACCESSION NUMBER: GenBank J02205.1
ggatcccagagtcaggggtctgtatcttcctgctggtggctccagttcaggaacagtaaaccctgctccgaatattgcctctcacatctcgtcaatctccgcgaggactggggaccctgtgacgaacatggagaacatcacatcaggattcctaggacccctgctcgtgttacaggcggggtttttcttgttgacaagaatcctcacaataccgcagagtctagactcgtggtggacttctctcaattttctagggggatctcccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatgggctacataattggaagttggggaactttgccacaggatc

SEQ ID NO: 330 - DNA, 65 bp, Artificial Sequence
  Synthetic construct
  ref <308>: DATABASE ACCESSION NUMBER: GenBank U34135.1
agcatacaatcaactatctggaattcccgaggactggggaccctgtgacgaacatggagaacatc

SEQ ID NO: 331 - protein, 151 aa, Artificial Sequence
  Artificially synthesized sequence
  ref <308>: DATABASE ACCESSION NUMBER: GenBank U34135.1
MFQDPQERPGKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 332 - DNA, 456 bp, Artificial Sequence
  Artificially synthesized sequence
  ref <308>: DATABASE ACCESSION NUMBER: GenBank M14923.1
atgtttcaggacccacaggagcgacccggaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 333 - protein, 179 aa, Artificial Sequence
  Artificially synthesized sequence
  ref <308>: DATABASE ACCESSION NUMBER: GenBank M14923.1
VNGPLFDHSTHSFAQPPNTALYYSVENVGLLPHLKEELARFIMGAGGSGADWAVSEFQKFYCFDGVSGITPTQRAAWRYIRELIIATTLFASVYRCGELELRRPDCSRPTSEGLYRYPPGVYLTYNSDCPLVAIVESGPDGCIGPRPVVVYDRDVFSILYSVLQHLAPRLAGGGTDAPP

SEQ ID NO: 334 - protein, 904 aa, Artificial Sequence
  Human alphaherpesvirus 2
  ref <308>: DATABASE ACCESSION NUMBER: GenBank AAA66440.1
MRGGGLICALVVGALVAAVASAAPAAPAAPRASGGVAATVAANGGPASRPPPVPSPATTKARKRKTKKPPKRPEATPPPDANATVAAGHATVRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYQATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVATCVPVAPDNVIVQNSMRVSSRPGTCYSRPLVSFRYEDQGPLIEGQLGENNDVRLTRDALEPCTVGHRGYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 335 - DNA, 3324 bp, Artificial Sequence
  Artificially synthesized sequence
gtcaacgggcccctgttcgaccactccacccacagcttcgcccagccccccaacaccgcgctgtactacagcgtcgagaacgtggggctcctgccgcacctcaaggaggaactcgcccgcttcatcatgggcgcggggggttcgggcgctgattgggccgtcagcgagtttcaaaagttctactgttttgacggtgtttccggaatcacgcccacccagcgcgccgcctggcgatatattcgcgagctcattatcgccaccacactctttgcgtcggtctaccggtgcggggagcttgagttgcgccgccccgactgcagccgcccgacctccgaaggtctgtaccgctacccgccgggcgtgtacctcacgtacaactccgactgtccgctggtggccatcgtcgagagcggccccgacggctgcatcggaccccgtccggtcgtggtttacgaccgagacgttttttccatcctctactcggtcctgcagcacctcgcccccagactagcgggcggcgggacggacgcgcccccgtaggcccgccatgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccaccgtgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccacgtacgccgccgaccgcttcaagcaggtcgacggcttctacgcgcgcgacctcaccacgaaggcccgggccacgtcgccgacgacccgcaacctgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactaccaggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtcgccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgacgtgcgcctcacccgcgacgcgctcgagccgtgcaccgtgggccaccgcggctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaagggaggggaggggagctgggcttgtgtataaataaaaagacaccgatgttcaaaaatacaca

SEQ ID NO: 336 - protein, 3 aa, Artificial Sequence
  Binding domain
  VARIANT 2: A natural number from 1-1000
GXC

SEQ ID NO: 337 - protein, 4 aa, Artificial Sequence
  Binding domain
  VARIANT 3: A natural number from 1-1000
GRXC

SEQ ID NO: 338 - protein, 4 aa, Artificial Sequence
  Binding domain
  VARIANT 3: A natural number from 1-1000
GKXC

SEQ ID NO: 339 - protein, 6 aa, Artificial Sequence
  Binding domain
  VARIANT 5: A natural number from 1-1000
GDGRXC

SEQ ID NO: 340 - protein, 6 aa, Artificial Sequence
  Binding domain
  VARIANT 5: A natural number from 1-1000
GEGRXC

SEQ ID NO: 341 - protein, 6 aa, Artificial Sequence
  Binding domain
  VARIANT 5: A natural number from 1-1000
GDGKXC

SEQ ID NO: 342 - protein, 6 aa, Artificial Sequence
  Binding domain
  VARIANT 5: A natural number from 1-1000
GEGKXC

SEQ ID NO: 343 - protein, 6 aa, Artificial Sequence
  Binding domain
  VARIANT 6: A natural number from 1-1000
EEEEEX

SEQ ID NO: 344 - protein, 7 aa, Artificial Sequence
  Binding domain
  VARIANT 7: A natural number from 1-1000
EEEEEEX

SEQ ID NO: 345 - protein, 6 aa, Artificial Sequence
  Binding domain
  VARIANT 6: A natural number from 1-1000
DDDDDX

SEQ ID NO: 346 - protein, 7 aa, Artificial Sequence
  Binding domain
  VARIANT 7: A natural number from 1-1000
DDDDDDX

SEQ ID NO: 347 - protein, 11 aa, Artificial Sequence
  Binding domain
FHCPYDLCHIL

SEQ ID NO: 348 - protein, 6 aa, Artificial Sequence
  His containing molecule
  VARIANT 5: A natural number from 1-1000
GDGRXH

SEQ ID NO: 349 - protein, 6 aa, Artificial Sequence
  His containing molecule
  VARIANT 5: A natural number from 1-1000
GEGRXH

SEQ ID NO: 350 - protein, 6 aa, Artificial Sequence
  His containing molecule
  VARIANT 5: A natural number from 1-1000
GDGKXH

SEQ ID NO: 351 - protein, 6 aa, Artificial Sequence
  His containing molecule
  VARIANT 5: A natural number from 1-1000
GEGKXH

SEQ ID NO: 352 - protein, 4 aa, Artificial Sequence
  His containing molecule
  VARIANT 3: A natural number from 1-1000
GRXH

SEQ ID NO: 353 - protein, 4 aa, Artificial Sequence
  His containing molecule
  VARIANT 3: Xaa = Any amino acid
GKXH

(19) United States
(12) Patent Application Publication
(10) Pub. No.: US 2018/0126001 A1
(43) Pub. Date: May 10, 2018

(54) ANTIBODY-VACCINE ENGINEERED CONSTRUCTS (AVEC)

(71) Applicant: Phoenix Biomolecular Engineering Foundation, San Francisco, CA (US)

(72) Inventors: Marek Malecki, San Francisco, CA (US); Roman Malecki, San Francisco, CA (US)

(21) Appl. No.: 15/620,679

(22) Filed: Jun. 12, 2017

Related U.S. Application Data
(60) Provisional application No. 62/349,394, filed on Jun. 13, 2016.

(51) Int. Cl. A61K 47/68; A61K 39/29; A61K 51/12; C07K 16/32; A61K 47/69; C07K 16/30

(52) U.S. Cl. CPC A61K 47/6851; A61K 39/292; A61K 51/1241; C07K 16/32; A61K 47/6923; C07K 16/30; A61K 2039/505; C07K 2317/54; C07K 2317/622; C07K 2317/569; C12N 2730/10123; C12N 2730/10134; C07K 2317/55

(57) ABSTRACT

Hereby, we disclose and claim, the concept, designs, enabling technologies, and utility for therapy of patients suffering from cancer, of a novel class of biomolecularly engineered, synthetic molecules: antibody-vaccine engineered constructs (AVEC). They comprise the main functional domains (antibodies and vaccines) and the supporting domains (linkers and reporters). Their mechanisms of actions rely upon antibody dependent redirecting, accelerating, and amplifying of the prophylactic or natural vaccination induced immune response (ADRAAVIR) from the initially elicited by vaccination towards the finally aimed by therapies. The routes of administration to the patients, pharmacokinetics, pharmacodynamics, pharmacogenomics, and therapeutic efficacies are resultant of those of the pertinent antibodies and vaccines assembled within AVEC.

### PRIORITY STATEMENT

This application claims priority to U.S. Provisional Patent Application No. 62/349,394, filed on 13 Jun. 2016, all of which are hereby incorporated herein by reference in their entirety as if fully set forth herein.

### REFERENCE TO A SEQUENCE LISTING

The present application incorporates by reference a Sequence Listing in electronic format submitted via EFS-Web. The Sequence Listing is provided as a file entitled PBEF-001A-Sequence-Listing created Oct. 25, 2017 which is 118,784 bytes in size. The information in the electronic format of the Sequence Listing is incorporated herein by reference in its entirety.

### BACKGROUND OF THE INVENTION

More than 1.5 million people will be newly diagnosed in the USA in 2017 [1]. Almost 600,000 people will die of cancer in the USA in 2017. Millions of people all over the world harbor early stage cancer without knowing it. Cancer is the number one killer for people under 85 [1].

Bleak survival statistics exist for many types of cancer. In 2010, the National Cancer Institute estimates that in the United States nearly 200,000 women will be diagnosed with breast cancer and 40,910 women will die of breast cancer. The 5 year survival for women diagnosed with stage I ovarian cancer (limited to ovaries) reaches 90%, but for women diagnosed with stage IV ovarian cancer (metastasized to distant organs) 5 year survival falls below 5%. [1] Prostate and lung cancer also have bleak survival statistics for patients with metastatic disease. Nearly 100% of patients diagnosed with stage I prostate cancer survive 5 years. However, as soon as the prostate cancer reaches stage III, the 5 year survival drops to 50%. The 5 year survival rate for stage I lung cancer patients is 50%, but stage IV patients have a 95% mortality rate over 5 years. These tragic statistics are largely a result of late diagnoses and inefficient, charged with iatrogenic adverse effects, of systemic therapeutics.

First line therapies of cancer include surgery, radiation therapy, and chemotherapy. Such therapies are aimed at reducing volumes of cancer cells, but they also inflict injuries to the healthy cells. Success of these therapies is dependent on early detection and aggressiveness of therapy, while limiting and/or providing remedies for the adverse effects. Hence, a very delicate balance between killing cancer cells and hurting the patients exists. That is monitored by the ratio between beneficial and iatrogenic effects of a therapy and by adjusting the therapeutic doses and regimens accordingly

Determining the appropriate therapy for a particular patients suffering from a particular cancer is very difficult. The patient's pharmacogenomics profile, as well as the therapeutics' Routes of Administration, Doses, Regimens (RDR), Absorption, Distribution, Metabolism, Excretion (Clearance) (ADME), Mechanism of action, Adverse effects, Toxicity, Resistance (MATR), Drug-Drug Interactions (DDI), Drug-Protein Interactions (DPI), Drug-Gene Interactions (DGI) all determine the success or failure of the designed and administered therapy. Hence, the vigorous studies conducted under the umbrella of personalized, precision medicine are of particular importance.

In this realm advances in prophylaxis, early diagnosis, and immune and gene therapy are of particular significance. Advances in therapy of cancer are the great examples of those.

Almost 30% of breast cancers overexpress genes Erbb-B2 aka HER-2; thus diagnosed as HER-2+ breast cancers. These cancers are associated with shorter times to relapses, as well as, shorter overall survivals, than those that did not overexpress Erb-B2 (HER-2). These data strongly support administering immunotherapy with antibodies against HER-2. [2-5]. Two domains of the HER-2 receptors are targeted by antibodies currently approved by the FDA: trastuzumab (Herceptin) and pertuzumab (Perjeta) and its modification pertuzumab-emtansine. [6, 7]. In combination with the M-phase specific systemic therapeutic-docetaxel, they result in an overall survival of more than 4.5 years, compared with 1.5 years achieved 14 years ago [8]. Mechanisms of action include: (I) inhibition of growth by steric inhibition of receptors' dimerization; (II) antibody-dependent cell-mediated cytotoxicity (ADCC); (III) complement dependent cytotoxicity (CDC) [8-11]. Therefore, efficacy of this immuno-therapy relies heavily upon engaging the patients' own immune system, as well as repressing resistance [12].

Ideally, the most effective way to reduce such a high incidence of breast cancers would be vaccination. Unfortunately, there are no breast cancer vaccines that are approved by the FDA. The clinical trials with various anti-cancer vaccines resulted in the overall efficacy in the range of 2.6% so far [13-14]. This is nowhere near the great efficacy of anti-viral and anti-bacterial vaccines, which are approved by the FDA and recommended by the CDC [15].

Vaccination against various microbes is the greatest achievement of the modern medicine. In particular, the vaccines against hepatitis B virus (HBV) are approved by the FDA and recommended by the CDC: Engerix B and Recombivax [16-18]. Measure of the immune system readiness is production of antibodies by immune cells at the titers strongly above 10.0 mIU/ml. If the antibody titer falls below that aforementioned value, the booster dose quickly reinvigorates the effective immunity.

Thanks to this program in the USA, incidence of Hepatitis B declined 82% over 17 years, i.e., from 8.5 cases per 100,000 population in 1990 to 1.5 cases per 100,000 population in 2007.

In clinical practice, we realized presence of a strange paradox. On one hand we have populations of patients, who are having their entire active adaptive immune system, enhanced due to the FDA approved prophylactic HBV vaccine, remaining on the stand-by. On the other hand, we have populations of patients, who have been diagnosed with cancers expressing and displaying qualitatively and/or quantitatively cancer specific molecules (e.g., HER-2, EGFR, EGFRvIII, CD20, CD52, CD44, CDv6, EpCAM, PSMA, etc)—as potential vaccination targets and who would greatly benefit from therapeutic vaccines, but who have not been provided and/or treated with prophylactic and/or therapeutic vaccines.

The aforementioned realization prompted our concept of designing of a class of artificial molecules (AVEC), which would be capable for functioning as a switch, so that vaccination induced immunity ready to eliminate invading microbes would be redirected to eliminate cancer cells.

Hereby, we disclose and claim the concept, designs, enabling technologies, and utility for therapy of cancer patients, of a novel class of biomolecularly engineered, artificial molecules: antibody-vaccine engineered constructs (AVEC), which do not occur in nature, were never designed/manufactured/published before, and which are not obvious.

Briefly, in practical molecular terms, we design and engineer: antibody-vaccine engineered constructs (AVEC) (e.g., comprising antibody anti-HER2 and vaccine against HBV and supporting molecules and linkers and reporters). AVEC are administered to the patients, who have become vaccinated/immunized against specific microbes (e.g., vaccines against HBV, HAV, HPV, HSV, CMV, EBV, etc) by means of medical vaccination and/or natural infection, but who have also developed cancers (e.g., HER-2+ Breast cancer, EGFR+ Colorectal cancer, CD20+ Lymphoma and Leukemia, PSMA+ Prostate cancer, CD52+ Leukemia, etc). Upon the AVEC administration to these patients, AVEC attach to the surfaces of cancer cells through the AVEC antibody domains (e.g., the anti-HER-2 antibody domain of AVEC guide AVEC molecules to HER-2+ Breast cancer cells). The patients' immune system does recognize, through the AVEC vaccine domains (e.g., vaccine against HBV, HPV, HSV, etc), the entire cancer-cell-AVEC complexes, as the insulting microbes (e.g., as the infecting HBV). This triggers antibody dependent redirected, accelerated, and amplified response (ADRAAVIR), which very efficiently and selectively eliminates cancer cells tagged with AVEC.

### BRIEF SUMMARY OF THE INVENTION

The antibody-vaccine engineered constructs (AVEC) are engineered to consist of the main functional domains (antibodies—guiding the AVEC to the targeted molecules and vaccines—eliciting innate and/or acquired immune response) and the supporting domains (linkers and reporters).

In one embodiment, an AVEC antibody main functional domains are provided—being AVEC guiding domains comprising cancer targeting antibodies including, but not limiting to an IgG, IgM, IgA, IgE, IgD classes of antibodies, Fab fragments, (Fab)2 fragments, single chain variable fragments (scFv), dual chain variable fragment (dcFv), single domain variable fragment (sdFv), single complementary regions (CDR), affibodies, aptamers including, but not limited to those against ErbB1-4, EGFRvIII, CD20, CD52, CD44, CD44v6, PSMA, EpCAM, TRA-1-60, TRA-1-81, SSEA-3, SSEA-4, CTLA4, etc.

In some embodiments, an AVEC vaccine main functional domains are provided—being AVEC immune response eliciting domain comprising all prophylactic and natural infection induced innate and adaptive immunity vaccines including, but not limiting: HBV, HPV, HCV, HAV, HEV, VZV, Polio virus, Mumps virus, Rubella virus, Measles virus, Rota Virus, Herpes simplex virus, Cytomegalovirus, Influenza virus, Rabies, Yellow fever virus, Dengue fever virus, Haemophilus influenzae, Diphteria, Pertusis, Tetanus, Tuberculosis, Cholera, Anthrax, Botulism, etc.

In some embodiments, an AVEC linking domains are provided—being a AVEC chemical bifunctional linker providing reactions, including, but not limited through the COOH, NH2 termini, lysine, cysteine, serine, threonine, tyrosine, aldehydes, ketones, azides, phosphines, phosphazides, thiazolidines, including, but not limited to employing SMBS, SMCC, EDC, PTAD, BMC, EDC, NHS esters, isothiocyanates, benzoyl fluorides, maleimides, iodoacetamides, thiopyridines, arylopropiolonitrile, diazonia, etc

In some embodiments, the AVEC linking domain is provided—being the amino acid (AA) sequence used to join antibody and vaccine through transgenic expression or chemical reaction involving bifunctional linkers to create integrated AVEC functional molecules.

In some embodiments, the AVEC linking domain is provided—being the DNA coding the AA sequences between antibody and vaccine domains, while/when expressed as the recombinant, fusion proteins or used as linkers.

In some embodiments, the AVEC reporters are provided—being the fluorochromes, magnets, bubbles, radionuclides, in the form of ions and/or nanoparticles, which serve as molecular lanterns facilitating localization with fluorescence, magnetic resonance, ultrasonography, positron emission tomography, gamma scintigraphy, computed tomography, of AVEC in vitro.

In some embodiments, the AVEC reporters are provided—being the fluorochromes, magnets, bubbles, radionuclides, in the form of ions and/or nanoparticles, which serve as molecular lanterns facilitating localization with fluorescence, magnetic resonance, ultrasonography, positron emission tomography, gamma scintigraphy, computed tomography, of AVEC in vivo in the patients' bodies.

In some embodiments, as required by the law, successful passing of the effects of AVEC upon two species, the AVEC are studied on humans. The two types of studies are conducted: in vitro ex vivo and in vivo.

In vitro ex vivo studies are conducted by establishing tissue cultures as models of in vivo tissues being encountered by AVEC. As reported above, in the peer-reviewed articles, Examples, and References, these human artery and vein endothelial cells, human normal breast tissues, human normal ovary surface cells, human normal prostate tissue, white blood cells, erythrocytes, platelets, etc. For the biopsies of these cells from the specific patients, the results of these studies can be included into the diagnostic process, while designing the dose and regimens of therapy, as the precision, personalized medicine.

In vivo studies involve procedures considered to be minimally invasive surgery: subcutaneous injections and intravenous infusions. They are always conducted by the physician.

### BRIEF DESCRIPTION OF SEVERAL VIEWS OF THE DRAWINGS

FIG. 1. Functional, molecular architecture of an antibody-vaccine engineered is illustrated. The main components of said AVEC are marked in capital letters: ANTIBODY, VACCINE, LINKER 1, LINKER 2, REPORTER. It is the front page view.

Said AVEC is comprising of said ANTIBODY: exemplified by an anti-HER-2; said VACCINE: an HBsAg; said REPORTER: an Apc (Allophycocyanin); said LINKER 1 (Gly4Ser1) and LINKER 2 (Gly4).

Said AVEC comprises of an ANTIBODY. Said ANTIBODY comprises of an anti-HER-2 FcHC-CDRs, anti-HER-2 LC-CDRs, C1q-DD, FcR-BD. Said anti-HER-2 HC CDR comprises a heavy chains' complementarity regions. Said CDR anti-HER-2 LC-CDRs comprises light chains' complementarity regions. Said C1q-DD comprises a complement systems' 1q docking domains. Said FcR-BD comprises a constant fragments' receptor binding domains.

Said AVEC comprises of VACCINE. Said VACCINE is exemplified by an HBsAg (illustrated by dark circle). Said HBsAg comprises of a human hepatitis B virus surface antigen virus like particle (VLP). Its purpose is to engage a native and to stimulate adaptive arms of immunity induced by the CDC required and the FDA approved common vaccination or raised after infection with an HBV. Both arms work as lightning rods for the entire immune system ascended down onto cancer cells, when assembled into AVEC.

Said LINKER 1 comprises of an integrating molecule (illustrated by bright lines between ANTIBODY and VACCINE) that joins said ANTIBODY with said VACCINE. Said LINKER 2 comprises an integrating molecule that joins said ANTIBODY with said REPORTER. Said 1 LINKER 1 may be either the same or different than said LINKER 2. Said LINKER 1 and LINKER 2 attain their functions by either a chemical conjugation or a genomic recombination. Said LINKER may be entirely absent in said AVEC expressed as a fusion protein.

Said REPORTER comprises: a fluorochrome, a superparamagnet, a nanoparticle, a radionuclide, a nanobubble (illustrated by a bright octagonal star). Said fluorochrome comprises of a fluorescent moiety facilitating its visibility (e.g., B phycoerythrin: Bpe); thus assessing pharmacokinetics of AVEC in vitro and in vivo. Said REPORTER may be entirely absent in said AVEC in absence of need for pharmacokinetic, pharmacodynamics, or imaging information.

FIG. 2A. Sensitivity and specificity of AVEC: anti-HER-2×HBsAg determined by nuclear magnetic resonance (NMR). The breast cancer cells were labeled with trastuzumab, anti-HER-2_{—001}×HBsAg, anti-HER-2_{—004}×HBsAg, anti-HBsAg (anti-HBsAg) and relevant isotype antibodies (iso) as indicated, followed by secondary superparamagentic antibodies. Measured changes in relaxivities reflect specificity and sensitivity of labeling. All experiments were conducted three times. The data presented are representative for all.

The SK-BR-3 [19] and the patients' breast cancer cells (BC001—the data for the patient 001 are representative to all 10 patients) cells were heavily labeled with trastuzumab, anti-HER-2 antibodies, and AVEC: anti-HER-2_{—001}×HBsAg, anti-HER-2_{—004}×HBsAg constructs.

The MCF-7 [20] and the patients' human breast epithelial=cells (HBE) were not labeled at the statistically significant range to cause relaxivity change. The isotype antibodies did not label the breast cancer and healthy cells.

FIG. 2B. Sensitivity and specificity of AVEC: anti-HER-2×HBsAg determined by immunoprecipitation and electrophoresis. Sera of patients with high titers of anti-HBsAg antibodies were rapidly cryoimmobilized, crushed, thawed, immunoprecipitated with superparamagnetic molecular baits as indicated, released into electrophoresis, and immunoblotted. Lanes' of superparamagnetic immunoprecipitation labels. 1. Immuno-naïve patient, not immunized, not infected, tested with the clinical diagnostics. 2. Vaccinated patient tested with AVEC: anti-HER-2_{—001}×HBsAg; 3 Vaccinated patient tested with isotype antibody; 4. Vaccinated patient tested with AVEC anti-HER-2_{—004}×HBsAg; 5. Vaccinated patient tested with isotype antibody; 6. Vaccinated patient tested with Engerix; 7. Vaccinated patient tested with isotype antibody; 8. Vaccinated patient tested with Recombivax; 9. Vaccinated patient tested with isotype antibody; 11. Vaccinated patient tested with anti-HBsAg isotype antibody 10, 12 molecular weight standards. Three experiments provided the same data. All molecular baits used in this study were pulling out the same anti-HBsAg molecule as determined by Hepatitis B clinical diagnostic assays.

FIG. 2C. Sensitivity and specificity of AVEC: anti-HER-2×HBsAg determined by immunoblotting. The blots from FIG. 2B were quantified at high sensitivity and revealed only anti-HBsAg antibodies in the patients' sera and no other molecules immunoprecipitated and labeled.

FIG. 2D. Sensitivity and specificity of trastuzumab determined by flow cytometry. The HER-2+ breast cancer cells were labeled with trastuzumab, tagged with fluorochrome (Tb) (intensity on X, count on Y in 1000/div.).

FIG. 2E. Sensitivity and specificity of AVEC: anti-HER-2_{—004}×HBsAg determined by flow cytometry. The HER-2+ breast cancer cells were labeled with AVEC: anti-HER-2_{—004}×HBsAg tagged with fluorochrome (Eu) (intensity on X, count on Y in 1000/div.).

FIG. 2F. Sensitivity and specificity of trastuzumab and AVEC: anti-HER-2×HBsAg determined by flow cytometry. The HER-2+ breast cancer cells were labeled with mix of both AVEC: anti-HER-2_{—004}×HBsAg (on X) and trastuzumab (on Y) tagged with different flurochromes outlined in FIG. 2D and FIG. 2E.

FIG. 2G. Sensitivity and specificity of anti-HER-2_{—001}×HBsAg determined by flow cytometry. The HER-2+ breast cancer cells were labeled with AVEC: anti-HER-2_{—001}×HBsAg followed by fluorescent anti-HBsAg.

FIG. 2H. Sensitivity and specificity of AVEC: anti-HER-2_{—001} determined by flow cytometry. The HER-2+ breast cancer cells were labeled with AVEC: anti-HER-2_{—001}×HBsAg followed by anti-HBsAg fluorescent antibodies on Y) and after initial labeling with AVEC: anti-HER-2_{—001}×HBsAg, the cells were labeled with the FITC fluorescent trastuzumab on X.

FIG. 2I. Sensitivity and specificity of AVEC: anti-HER-2_{—001}×HBsAg determined by flow cytometry. After initial labeling with AVEC: anti-HER-2_{—001}×HBsAg, the cells were labeled with the FITC fluorescent trastuzumab (on X) for the cell count (on Y).

FIG. 2J. Sensitivity and specificity of trastuzumab determined by flow cytometry. The HER-2+ breast cancer cells were labeled with isotype antibodies for trastuzumab.

FIG. 2K. Sensitivity and specificity of AVEC: anti-HER-2_{—001}×HBsAg determined by flow cytometry. The HER-2+ breast cancer cells were labeled with isotype antibodies for isotype antibodies for anti-HER-2_{—001}.

FIG. 2L. Sensitivity and specificity of AVEC: anti-HER-2_{—004}×HBsAg determined by flow cytometry. The HER-2+ breast cancer cells were labeled with isotype antibodies for isotype antibodies for anti-HER-2_{—004}.

FIG. 2M. Sensitivity and specificity of AVEC: anti-HER-2×HBsAg determined by flow cytometry. The HER-2+ breast cancer cells were labeled with isotype antibodies for isotype antibodies for isotype antibodies for anti-HBsAg.

FIG. 3A. Mechanism of action of AVEC: anti-HER-2×HBsAg. determined by thymidine incorporation. SK-BR-3 breast cancer cells were labeled for 1-7 h at 37° C. with 0.3 mg/ml trastuzumab or anti-HER-2×HBsAg in full human sera from the HBV-vaccinated patients keeping strongly above 100.0 IU/ml anti-HBV. That was followed by studying growth inhibition through thymidine incorporation.

FIG. 3B. Mechanism of action of AVEC: anti-HER-2×HBsAg determined by immunolabeling and fluorescence microscopy. The breast cancer cells from the patient with metastatic HER-2+ breast cancer were treated with fluorescent AVEC: anti-HER-2_{—001}×HBsAg. These tests were repeated three times. This route of MOA is representative for all 10 patients' breast cancer cells.

FIG. 3C. Mechanism of action of AVEC: anti-HER-2_{—001}×HBsAg determined by labeling with anti-phosphatidylserine (anti-PS) to detect apoptosis. The breast cancer cells from the patient with metastatic HER-2+ breast cancer were treated with fluorescent anti-phosphatidylserine (anti-PS). These tests were repeated three times. This route of MOA is representative for all 10 patients' breast cancer cells. After 1 h some cells were slipping into apoptosis as shown by the PS flip.

FIG. 3D. Mechanism of action of AVEC: anti-HER-2_{—001}×HBsAg determined by labeling with propidium iodide (PI) to detect necrosis. The breast cancer cells from the patient with metastatic HER-2+ breast cancer were treated with PI. These tests were repeated three times. This route of MOA is representative for all 10 patients' breast cancer cells. After 1 h the cells were showing collapse of chromatin.

FIG. 3E. Mechanism of action of AVEC: anti-HER-2_{—001}×HBsAg determined by immunoassay. After 6 h most of the cells, which were marked as apoptotic by anti-PS. Almost 40% of the cancer cells were apoptotic due to treatment. That efficacy more than doubled after the treatment with anti-HER-2₀₀₁×HBsAg or anti-HER-2₀₀₄×HBsAg. Apoptotic cells: AC.

FIG. 3F. Mechanism of action of AVEC: anti-HER-2_{—001}×HBsAg determined by immunoassay. Nearly 10% were determined necrotic as the result of the treatment with trastuzumab. The percentage of the necrotic cells after the treatment with anti-HER-2₀₀₁×HBsAg or anti-HER-2₀₀₄×HBsAg more than tripled over that attained with trastuzumab. Necrotic Cells: NC.

FIG. 4A. Factors affecting immunotherapeutic efficacy of AVEC: anti-HER-2×HBsAg.

SK-BR-3, MCF-7, and the patients' HER-2+ breast cancer cells were treated with trastuzumab, biosimilar anti-HER-2, anti-HBV, AVEC: anti-HER-2₀₀₁×HBsAg, and AVEC: anti-HER-2_{—0041}×HBsAg in erythrocyte-free blood, in which concentrations of the complement system were adjusted according to measuring of C1q and C3 at 37° C. The experiments were concluded by labeling of the cells with propidium iodide. Necrotic cells were counted by flow cytometry. The experiments were repeated three times. Increasing concentrations of complement system components resulted in increased efficacy of the breast cancer cells killing. The novel immunotherapeutics AVEC: anti-HER-2₀₀₄×HBsAg and AVEC: anti-HER-2₀₀₁×HBsAg more than doubled the efficacy trastuzumab and anti-HER-2 biosimilars via the CDC. KC: killed cells. CC: C1/C3 complement concentration ratios.

FIG. 4B Factors affecting immunotherapeutic efficacy of AVEC: anti-HER-2₀₀₄×HBsAg and AVEC: anti-HER-2₀₀₁×HBsAg. SK-BR-3, MCF-7, and the patients' breast cancer cells were treated with trastuzumab, anti-HER-2, anti-HBV, and AVEC: anti-HER-2₀₀₄×HBsAg and AVEC: anti-HER-2₀₀₁×HBsAg in erythrocyte-free blood, in which the number of the immune cells was adjusted in relation to the number of breast cancer cells. Measurements were pursued as in FIG. 3A. Increasing the ratio of the effector immune cells to target cancer cells resulted in proportional increase in efficacy of the breast cancer cells' killing. The novel clusters AVEC: anti-HER-2₀₀₄×HBsAg and AVEC: anti-HER-2₀₀₁×HBsAg more than tripled the efficacy of the experimental therapy via ADCC over trastuzumab and anti-HER-2 biosimilars. KC: killed cells. CC: effector cells/cancer cells concentration ratios.

FIG. 5A. Sensitivity and specificity of AVEC: anti-HER-2_{—001}×HBsAg in targeting ovarian cancer cells determined by nuclear magnetic resonance. The ovarian cancer cells from cultures (OV90, TOV112, SK-OV-3), human ovary surface (HOSE) cells were labeled with trastuzumab, AVEC: anti-HER 2_{—001}×anti-HBsAg, AVEC: anti-HER-2_{—004}×anti-HBsAg, and relevant isotype antibodies rendered superparamagnetic. Antibodies labeling the cells were changing the cells' magnetic properties, which were measured with nuclear magnetic resonance (NMR). The assays were repeated four times. The data presented are representative for all acquired. The changes in the length of T1 resulted from the changes in magnetic resonance, which were altered by presence of superparamagnetic antibodies and were proportional to the numbers of antibodies attached to the labeled cells. With the same number of cells in each batch, the relaxivity changes were directly proportional to the numbers of cell receptors displayed by the cells. Therefore, they facilitated comparisons of sensitivity of labeling between the cells, while being pursued with different antibodies. The OV90, TOV112, and SKOV3 cells were labeled with trastuzumab, anti-HER-2 biosimilars, AVEC: anti-HER-2_{—001}×anti-HBsAg and AVEC: anti-HER-2_{—004}×anti-HBsAg at the statistically significant superiority over those labeled with the isotype antibodies and isotype-based AVEC. The control HOSE cells were labeled at the same negligible levels as the isotype antibodies. (i: isotype antibody).

FIG. 5B Sensitivity and specificity of AVEC: anti-HER-2×HBsAg in targeting ovarian cancer patients determined by nuclear magnetic resonance. The ovarian cancer ascites cells from the patients (Patient 1-Patient 3) were labeled with the same superparamagnetic antibodies, followed by the measurement of relaxivities, as outlined for cultured cells in FIG. 5A. The ovarian epithelial cancer ascites cells were all showing high numbers of the HER-2 receptors specifically labeled by the tested antibodies. Measurements of the relaxivities demonstrated statistically significant difference in the numbers of receptors on the ovarian epithelial cancer ascites cells over HOSE. Measurements of the relaxivities demonstrated statistically significant difference attained by labeling with the anti-HER-2 antibodies and AVEC over the relevant isotypes. The assays were repeated four times. The data presented are representative for all acquired (i isotype antibody).

FIG. 6A. Sensitivity and specificity of trastuzumab to attract anti-Fc antibodies. The ovarian cancer cells from cultures (OV90) were labeled initially with trastuzumab followed by the anti-FcR-BD fluorescent antibody (N: cell count on Y; intensity on X).

FIG. 6B Sensitivity and specificity of trastuzumab to attract anti-HBV antibodies. The ovarian cancer cells from cultures (OV90) were labeled initially with trastuzumab followed by the anti-HBV fluorescent antibody (F2: on X) and trastuzumab isotype followed by anti-Fc antibody (F1: on Y). None of them resulted in labeling of HER-2+ ovarian cancer cells.

FIG. 6C Sensitivity and specificity of AVEC: anti-HER-2_{—001}×HBsAg to attract anti-Fc antibodies. The ovarian cancer cells from cultures (OV90) were labeled initially with AVEC: anti-HER-2_{—001}×HBsAg followed by the anti-FcR-BD fluorescent antibody (N: cell count on Y; intensity on X).

FIG. 6D. Sensitivity and specificity of AVEC: anti-HER-2_{—001}×HBsAg to attract anti-HBV antibodies. The ovarian cancer cells from cultures (OV90) were labeled initially with AVEC: anti-HER-2_{—001}×HBsAg followed by THE anti-HBsAg fluorescent antibody. The assays were repeated four times. The data presented are representative for all acquired. These cells revealed having high number of HER-2 receptors (>2×106/cell) were effectively labeled with AVEC: anti-HER-2_{—001}×HBsAg and anti-HBV antibodies. However, only the HER2+ ovarian cancer cells labeled with AVEC attracted the anti-HBsAg antibody onto the ovarian cancer cells, but the cells labeled with trastuzumab did not not attract anti-HBV antibodies.

FIG. 6E. Sensitivity and specificity of AVEC: anti-HER-2_{—001}×HBsAg to attract anti-HBV antibodies. Blood from the patients (1-3) vaccinated against HBV were depleted of erythrocytes, magnetic AVEC's: anti-HER-2₀₀₁×HBsAg were used to pull out anti-HBsAg, while the concentrations were adjusted to 10.0 mIU/ml and electrophoresed. Strong bands corresponding to molecular weight of anti-HBsAg antibodies are clear.

FIG. 6F. Sensitivity and specificity of AVEC: anti-HER-2_{—001}×HBsAg to attract anti-HBV antibodies. The gels from FIG. 6E were quantified. These data show very specific and sensitive affinity of the patients' anti-HBsAg towards AVEC: anti-HER-2_{—001}×HBsAg. Some of the antibodies were undergoing some degree of chain separation showing up as faster bands.

FIG. 6G. Sensitivity and specificity of AVEC: anti-HER-2_{—001}×HBsAg to attract anti-HBV antibodies. Blood from the patients (1-8) vaccinated against HBV were depleted of erythrocytes, magnetic AVECs: anti-HER-2_{—001}×HBsAg were used to pull out anti-HBsAg and electrophoresed without adjusting the concentrations.

FIG. 6H. Sensitivity and specificity of AVEC: anti-HER-2_{—001}×HBsAg to attract anti-HBV antibodies. The gels from FIG. 6G were quantified. The quantifications revealed different concentrations of the anti-HBsAg in different patients and different degree dissociation of the chains. The assays were repeated four times. The data presented are representative for all acquired.

FIG. 7A. Mechanism of action of AVEC: anti-HER-2_{—001}×HBsAg in ovarian cancer cells' killing determined by immunofluorescence. The patients' ovarian cancer cells were labeled for 6 h at 37° C. with AVEC: anti-HER-2_{—001} in full erythrocytes' free blood from the HBV-vaccinated patients with the anti-HBV adjusted to 10.0 IU/ml. That was followed by labeling with anti-phosphatidylserine (anti-PS). Most of the cells demonstrate a flip of phosphatidylserine; thus advancing apoptosis. The assays were repeated four times. The data presented are representative for all acquired.

FIG. 7B. Mechanism of action of AVEC: anti-HER-2_{—001}×HBsAg in ovarian cancer cells' killing determined by immunofluorescence. The patients' ovarian cancer cells were labeled for 6 h at 37° C. with AVEC: anti-HER-2_{—001}×HBsAg in full erythrocytes' free blood from the HBV-vaccinated patients with the anti-HBV adjusted to 10.0 IU/ml. That was followed by labeling with the fluorescent anti-genomic DNA anti-gDNA). The labeling is indicative of advancing necrosis. The assays were repeated four times. The data presented are representative for all acquired.

FIG. 7C. Mechanism of action of AVEC: anti-HER-2_{—001}×HBsAg and anti-HER-2_{—004}×HBsAg in repressing the ovarian cancer cells' growth was studied by radiolabeling. That was accomplished by incorporation of tritium tagged thymidine. AVEC inflicted statistically significant higher impact upon the cells' growth, when compared to isotype antibodies. AVEC had negligible effects upon HAE and HOSE cells.

FIG. 7D. Mechanism of action of AVEC: anti-HER-2_{—001}×HBsAg byinduced apoptosis was evaluated by labeling with anti-phosphatidylserine (anti-PS) superparamagnetic antibodies and measuring relaxivity in NMR. Anti-HER-2 naked antibodies resulted in approximately 40% of cells being apoptotic. Treatment with the AVEC resulted in the number of apoptotic cells more than doubled. Labeling of HOSE cells was negligible. Labeling of cells with isotype antibodies (i) was negligible.

FIG. 7E. Mechanism of action of AVEC: anti-HER-2_{—001}×HBsAg by induced necrosis was evaluated by labeling with anti-genomic DNA (anti-gDNA) superparamagnetic antibodies and measuring relaxivities in NMR. Anti-HER-2 naked antibodies resulted in approximately 10% of cells being necrotic. Treatment with the AVEC: anti-HER-2_{—001}×HBsAg resulted in the number of necrotic cells nearly tripled over anti-HER2 biosimilar. These assays were repeated four times. The data presented are representative for all acquired.

FIG. 8A. Factors affecting immunotherapeutic efficacy of AVEC: anti-HER-2_{—001}×HBsAg.

As the controls, blood of every healthy volunteer and HBV infected and vaccinated patients was depleted of erythrocytes and analyzed. Three main populations of cells were revealed by forward (FS) and side (S) scattering. The data presented are representative for all acquired.

FIG. 8B. Factors affecting immunotherapeutic efficacy of AVEC: anti-HER-2_{—001}×HBsAg. As the controls, blood of the every healthy volunteer and HBV infected and vaccinated patients was depleted of erythrocytes and analyzed. Various cells fractions (I) were determined through cell counts.

FIG. 8C. Factors affecting immunotherapeutic efficacy of AVEC: anti-HER-2_{—001}×HBsAg. As the controls for the numbers of NKC and CTL effector cells, various fractions of white blood cells (WBC) labeled with anti-FcR were sorted and counted, so that the number of the effector cells and cancer cells to effector cells inter-fractions' ratios could be adjusted in the forthcoming experiments and personalizing therapy.

FIG. 8D. Factors affecting immunotherapeutic efficacy of AVEC: anti-HER-2_{—001}×HBsAg promoting ADCC. The OV90 cells were labeled with AVEC: anti-HER-2_{—001}×HBsAg followed by anti-Fc-R-BD on Y as F1 and anti-HBsAg (on X as F4). These assays were repeated four times. The data presented are representative for all performed.

FIG. 8E Factors affecting immunotherapeutic efficacy of AVEC: anti-HER-2_{—001}×HBsAg via complement system (CS). are OV90 cells were treated at 37° C. with trastuzumab, biosimilar anti-HER-2, anti-HBV, AVEC: anti-HER-2_{—004} and AVEC: anti-HER-2_{—001}×HBsAg in erythrocyte-free blood from the HBV vaccinated patients, while the concentrations of the complement system (CS) were adjusted for C1q and C3 as indicated on the diagram. Increasing concentrations of complement system components resulted in increased efficacy of the ovarian cancer cells killing counts (KC) as complement dependent cytotoxicity (CDC). Importantly, treatment with AVEC at nearly three times lower concentrations of C1q and C3 resulted in nearly the same therapeutic efficacy as naked anti-HER-2 antibodies at three times higher concentrations. This efficacy was at significantly higher statistical rate than with the relevant isotype antibodies. This impact onto cancer cells was also of statistical significance difference over that onto human ovary surface epithelial (HOSE) and human artery endothelial HAE cells. These assays were repeated four times. The data presented are representative for all performed. KC: killed cells. CC: C1/C3 complement concentration ratios.

FIG. 8F. Factors affecting immunotherapeutic efficacy of AVEC: anti-HER-2_{—004} and AVEC: anti-HER-2_{—001}×HBsAg via effector cells. OV90 cells were treated at 37° C. with trastuzumab, biosimilar anti-HER-2, anti-HBV, and AVEC:anti-HER-2₀₀₄ and AVEC: anti-HER-2_{—001}×HBsAg in erythrocyte-free blood from the HBV vaccinated patients, while the ratios between cytotoxic effector cells (EF) and the killed ovarian cancer cells (KC) were adjusted as indicated on the diagram. Increasing the ratio of the effector cytotoxic cells to ovarian cancer cells clearly increased efficacy of killing cancer cells by antibody dependent cytotoxic cells (ADCC). Importantly, ratios of effector to cancer cells, when AVEC were administered, resulted in the same immunotherapeutic efficacy as compared to higher ratios when naked antibodies were administered. In other words less cytotoxic cells were needed for AVEC to deliver the same therapeutic effect as more cells when naked antibodies were administered. This feature is critically important, when the patients are immunocompromised after the rounds of systemic therapy and the patients' ability grow the immune cells is annihilated by intended to suppress proliferation of cancer cells, but as side effects universally suppressing proliferation of all patients' cells. The AVEC's efficacy was at statistically significant advance over the relevant isotype antibodies. The AVEC's impact onto cancer cells was also of statistically significant difference over that onto HOSE and HAE cells. These assays were repeated four times. The data presented herein are representative for all acquired. KC: killed cells. EF: effector cells: cancer cell numbers' ratios.

### DETAILED DESCRIPTION OF THE PREFERRED EMBODIMENT

### Antibody-Vaccine Engineered Constructs (AVEC)

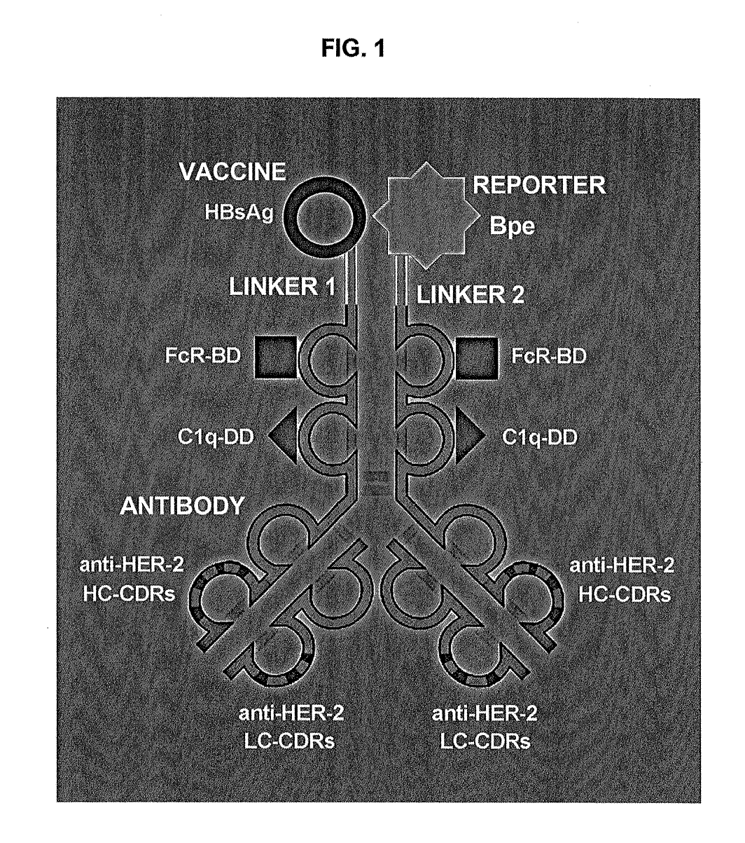

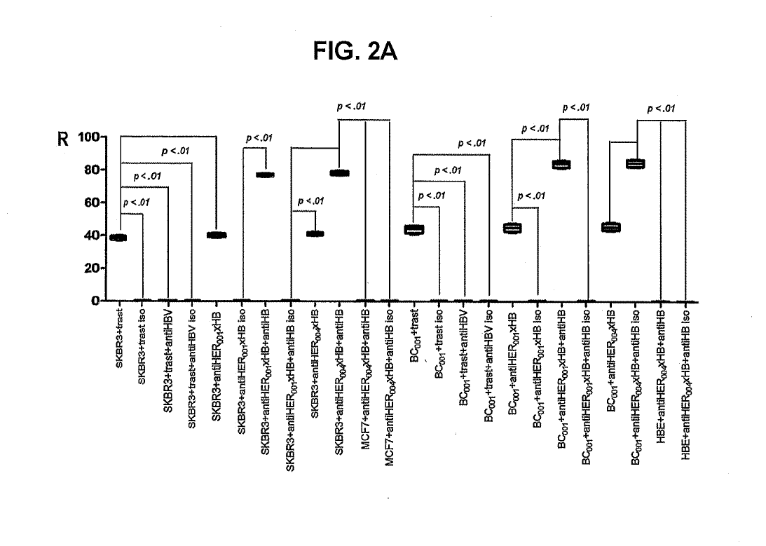

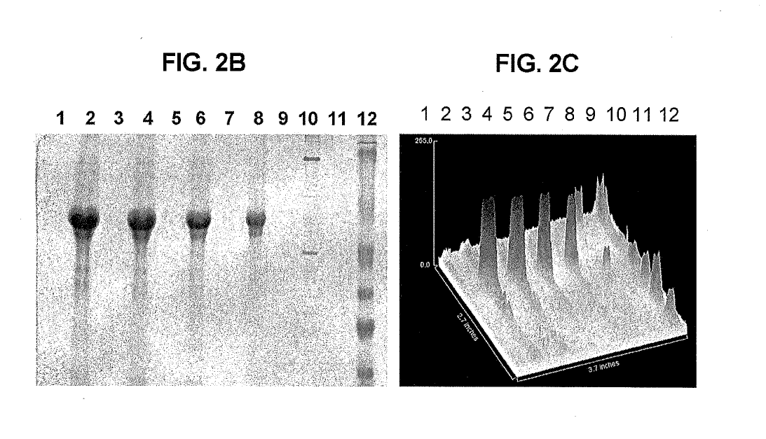

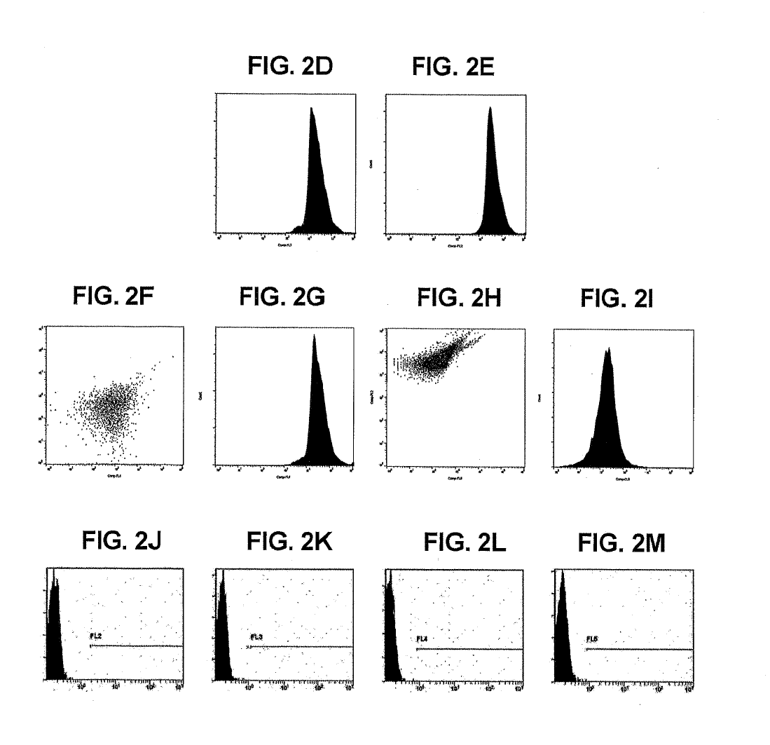

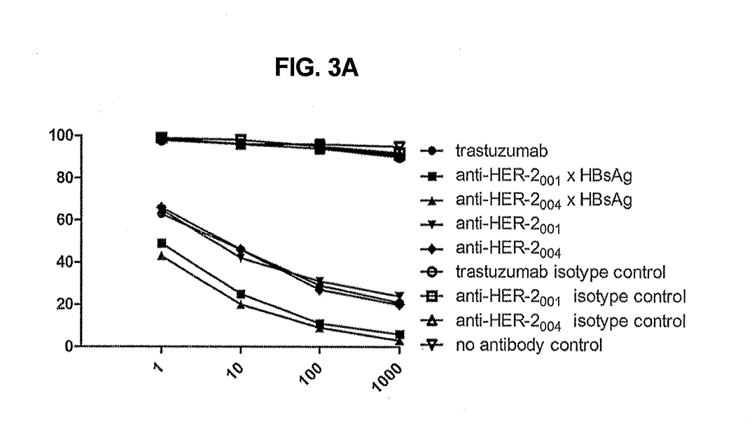

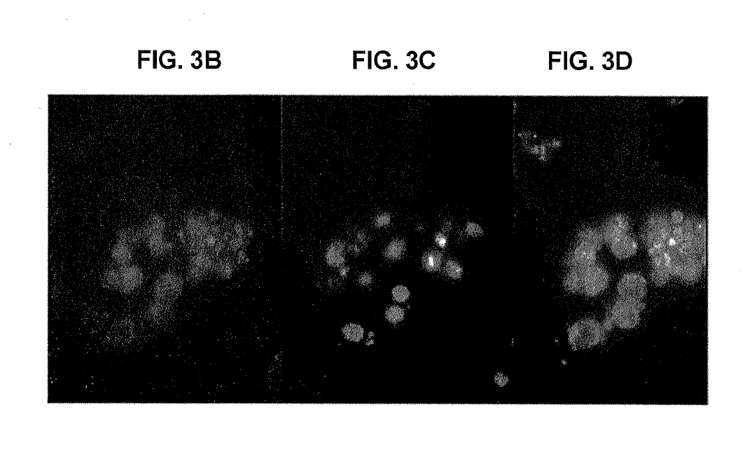

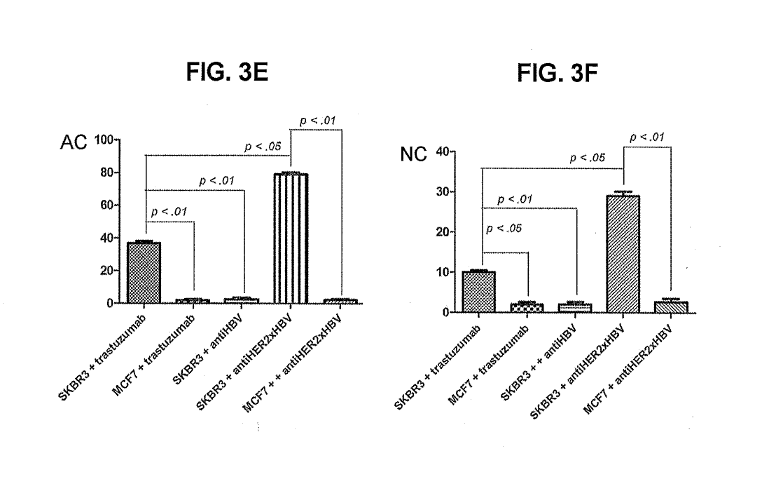

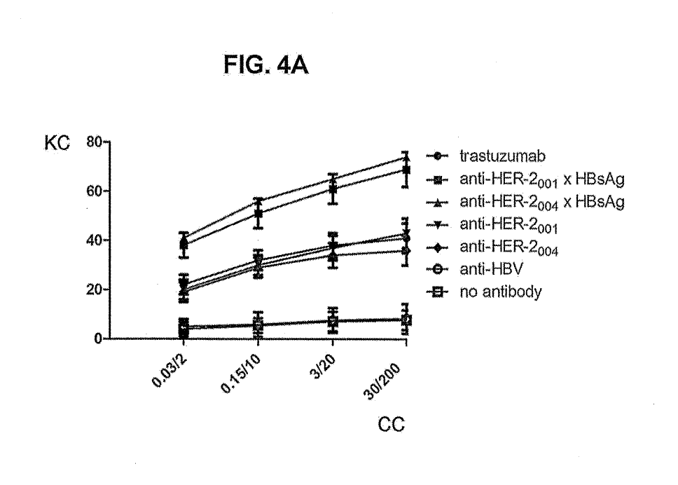

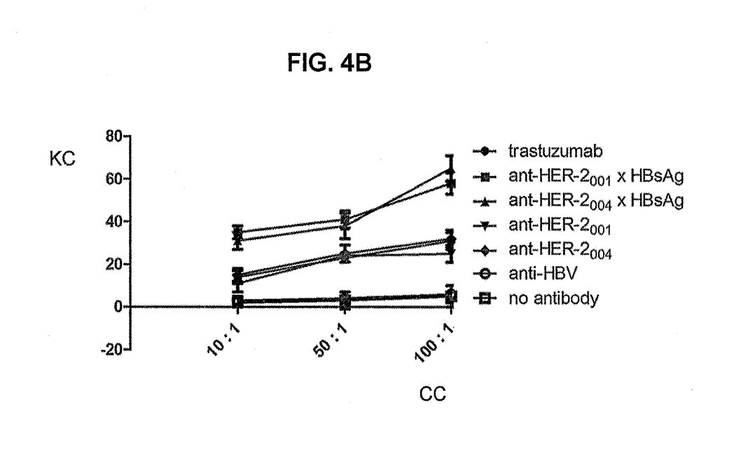

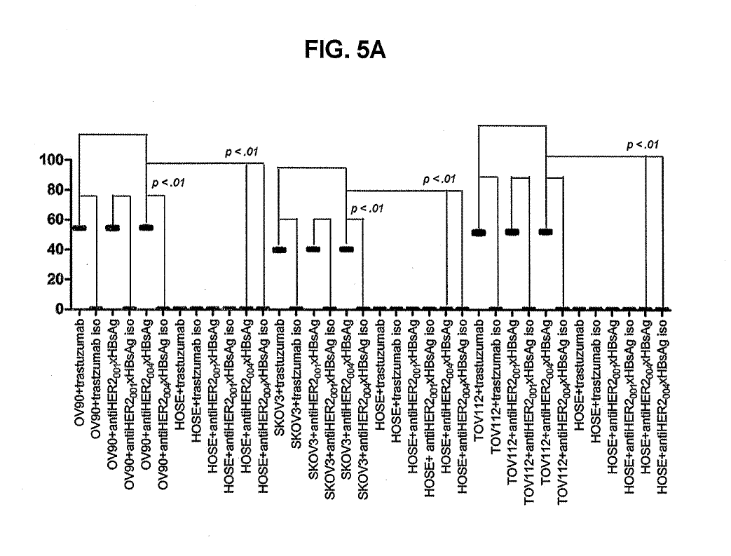

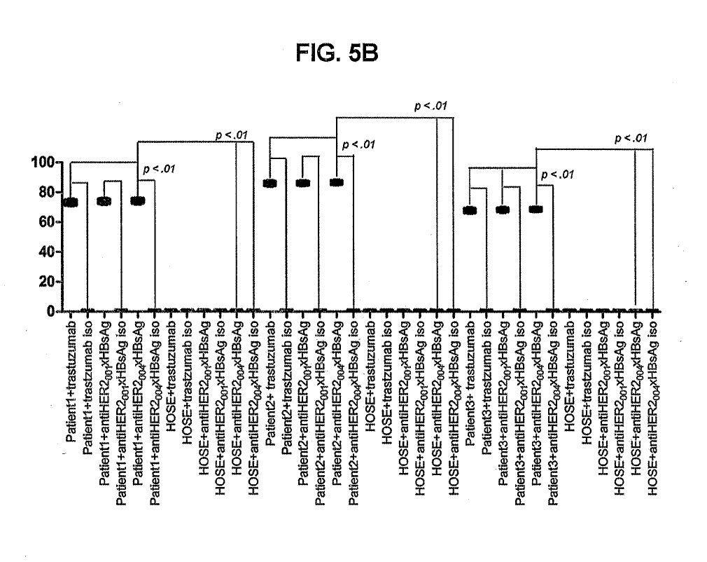

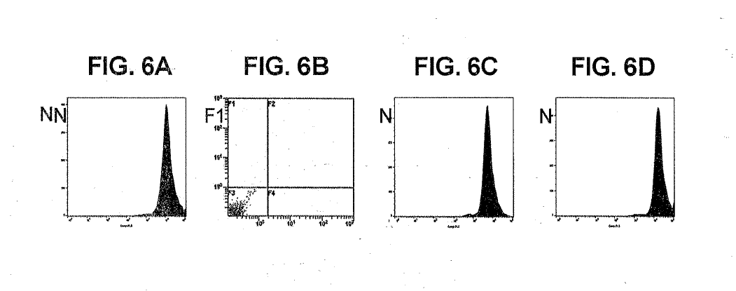

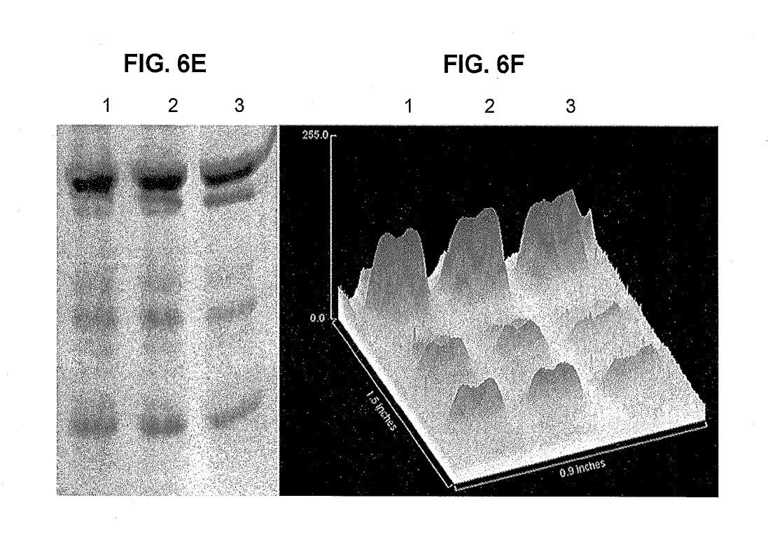

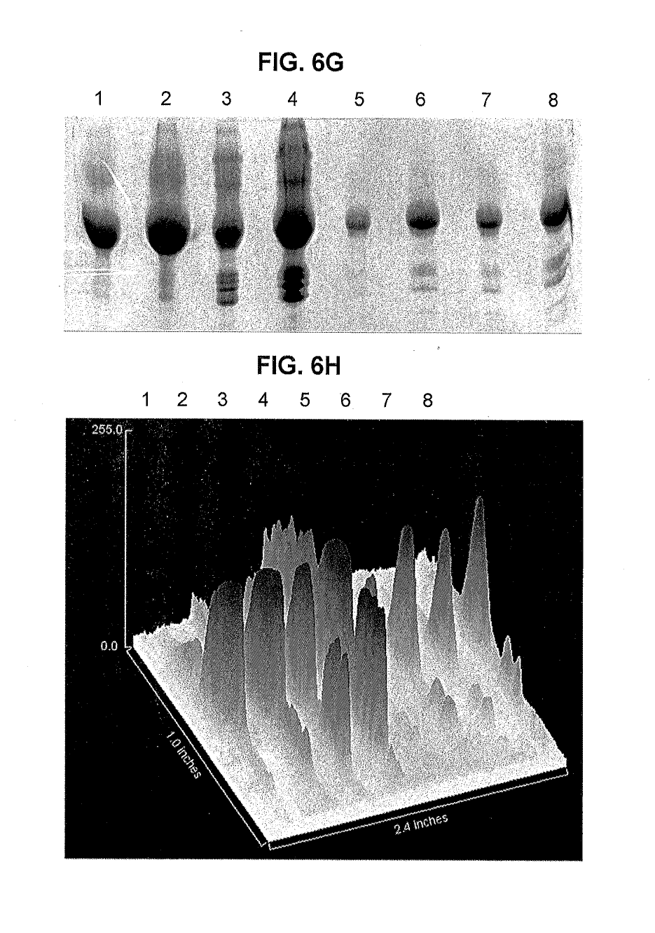

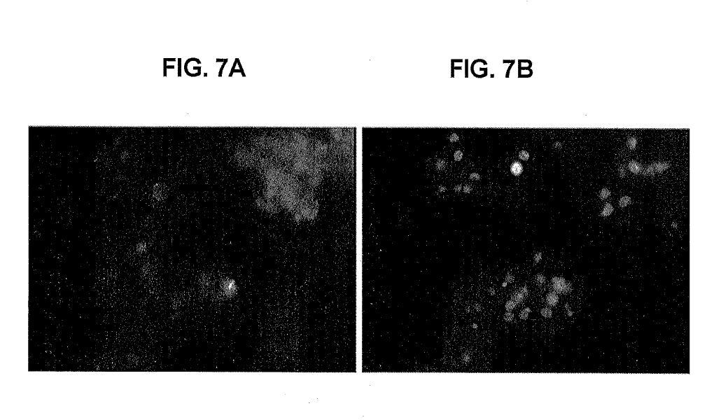

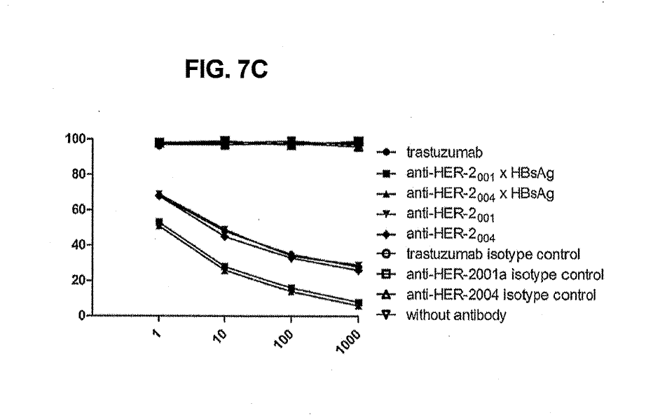

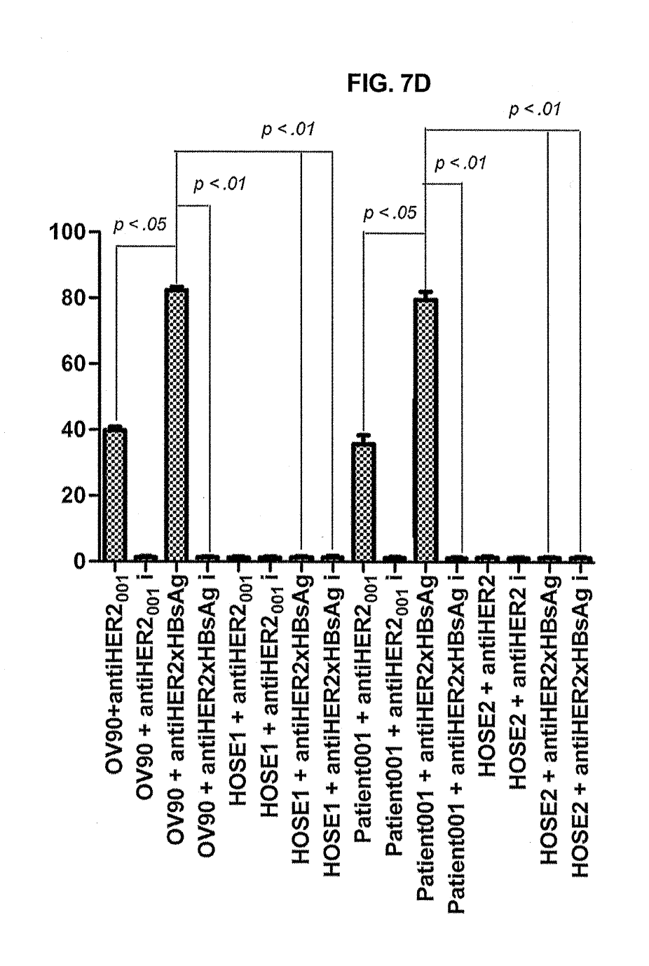

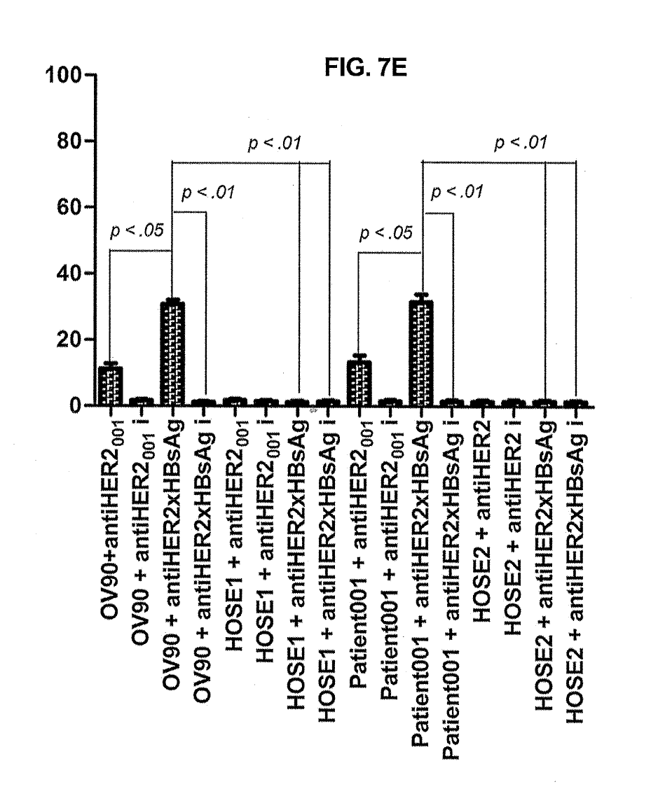

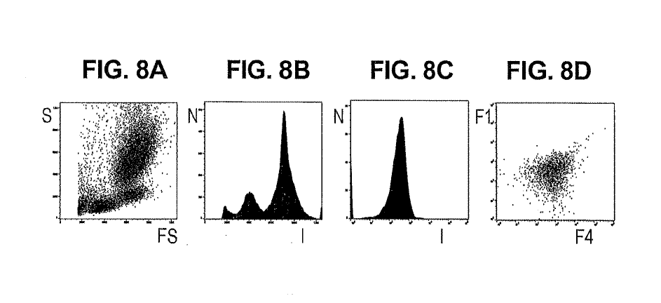

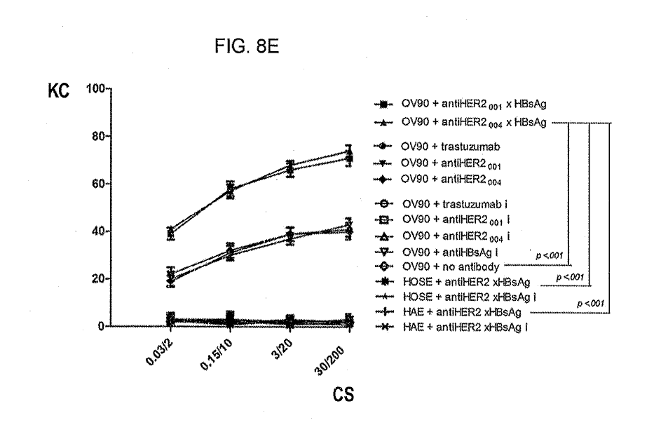

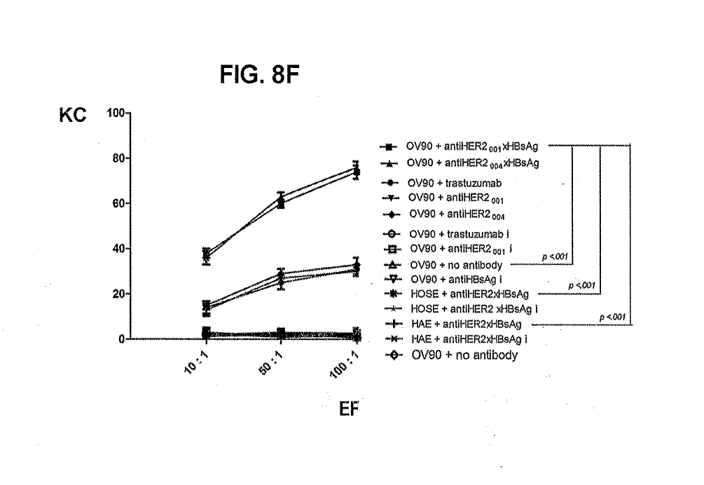

An antibody-vaccine engineered construct (AVEC) comprising of an ANTIBODY, a VACCINE, a LINKER, a REPORTER is illustrated in FIG. 1. Furthermore, the methods for designing and manufacturing of AVEC, as in claim 1, are provided herein. Moreover, the utility of said AVEC, as in claim 1, for therapy of cancer is provided herein. Finally, the methods for determination of their pharmacokinetics, pharmacodynamics, and pharmacogenomics of said AVEC, as in claim 1, as pertinent to determining the personalized, precision medicine designing of effective routes of administration, doses and regimens, as well as, verifying their clinical efficacy, are provided herein.

AVEC described herein, as designed, generated, and tested, as described in the Examples below and References have a very high qualitative and/or quantitative specificity towards biomarkers on tumor cells or cancer cells in vivo or in vitro, while in blood, serum, or any other physiological solution, fluid and/or tissue. While having the very high binding specificity and sensitivity to tumor cell or cancer cell biomarkers, AVEC have almost no specificity and sensitivity, toxicity towards the healthy cells. They are entirely bio-compatible. They have very long shelf-lives.

Their mechanisms of actions, as in the details described and analyzed in the Examples below, rely upon antibody dependent redirecting, accelerating, and amplifying of the prophylactic or natural vaccination induced immune response (ADRAAVIR) from that initially elicited by vaccination towards this finally aimed by therapies=redirecting immunity from microbes against cancers.

Their route of administration to the patients, pharmacokinetics, pharmacodynamics, and pharmacogenomics, as described in the Examples below, are resultant of those of the pertinent antibodies and vaccines assembled within AVEC.

Therefore, we disclose an entirely new and a very broad and entirely novel paradigm for immunotherapy of patients suffering from cancer and autoimmune diseases, which has become viable through biomolecular engineering of artificial molecules, which are capable for interfacing preventive and therapeutic immunities.

Development of two immunotherapeutics: trastuzumab and pertuzumab, against the same target HER-2, is de facto an attempt of reconstructing the response of the natural immune system involving polyclonal antibodies. Herein, we present a way to by-pass the need for developing multiple clones of antibodies against HER-2, but rather we use HBsAg, which serves as a lightning rod for redirecting all the clones of antibodies and cytokines generated by the FDA approved and the CDC recommended prophylactic immunization. Therefore, it amplifies the therapeutic efficacy of the single clone of anti-HER-2 to the level equivalent to eliciting polyclonal antibodies, plethora of cells, and multiple cytokines.

If ever developed, any new anti-cancer vaccines would have to be introduced against multiple cancers and assembled into a program, as now by the CDC for microbes: viruses and bacteria. Otherwise, development of the active, strong immune response by a patient suffering from a cancer and undergoing cytocidal and toxic systemic therapies, whose immune system is greatly compromised by the first line systemic chemotherapies, is hard. Systemic therapies are intended to kill all proliferating cancer cells, but as the omnipresent adverse effect, they result in immunocompromised patients, as the proliferating cells of the immune system are killed.

Moreover, multiple vaccinations are multiple immunizations; thus each additional one of them may increase risks of cross reactivity.

In some embodiments, as outlined in one of Examples provided below, AVEC engage both arms of the patients' immune system: innate and adaptive—acquired through vaccination against e.g., hepatitis B virus. HBsAg is the epitope uniquely different from all epitopes displayed on healthy human cells. Furthermore, this therapy—ADRAAVIR does not rely upon time consuming development of a new therapeutic response, when in fighting of cancer, the time is of essence; acceleration of therapy is of essence, as every day the cancer progresses, if not stopped. Moreover, ADRAAVIR relies upon existing readiness of immune system of the patients. As such, ADRAAVIR presents the least iatrogenic, fastest, most efficient option for immunotherapy of cancer patients.

### Antibody Domains:

Efficacy and safety of therapy with AVEC rely upon specificity and sensitivity of their antibody domains. The very specific pharmacogenomics tests are required by the FDA to be performed prior to administration of the immunotherapy employing naked antibodies, antibody drug conjugates (ADC), and antibody toxin conjugates (ATC). These tests are aimed to determine, if receptors being the potential targets of antibodies are overexpressed on cancer cells. This is accomplished by PCR, FISH, and/or IHC. Accordingly, we have assembled the panel of probes and recombinant isolated receptors for measuring the level of expression of the therapeutic targets for AVEC. Some of them including, but not limiting, to those shown in the table below.

TABLE
PRIMERS AND PROBES TO ERBB 1-4.

| | OLIGO | len | tm | gc% | any | 3 | seq |
|---|---|---|---|---|---|---|---|
| ErbB1 | LEFT PRIMER | 20 | 60.01 | 50.00 | 6.00 | 1.00 | Cagcgctaccttgtcattca (SEQ ID |
| | RIGHT PRIMER | 20 | 60.00 | 55.00 | 7.00 | 2.00 | NO: 1) Tgcactcagagagctcagga |
| | HYB OLIGO | 20 | 60.08 | 45.00 | 8.00 | 3.00 | (SEQ ID NO: 2) |
| | | | | | | | gaatgcatttgccaagtcct (SEQ ID |
| | | | | | | | NO: 3) |
| ErbB1 | LEFT PRIMER | 20 | 60.00 | 55.00 | 3.00 | 1.00 | gggctcacagcaaacttctc (SEQ ID |
| | RIGHT PRIMER | 20 | 60.02 | 50.00 | 7.00 | 0.00 | NO: 4) aagccagactcgctcatgtt |
| | HYB OLIGO | 20 | 60.00 | 55.00 | 2.00 | 2.00 | (SEQ ID NO: 5) |
| | | | | | | | acacacacacacacacaccg (SEQ ID |
| | | | | | | | NO: 6) |
| ErbB1 | LEFT PRIMER | 20 | 60.00 | 55.00 | 3.00 | 1.00 | ggctcacagcaaacttctcc (SEQ ID |
| | RIGHT PRIMER | 20 | 60.02 | 50.00 | 7.00 | 0.00 | NO: 7) aagccagactcgctcatgtt |
| | HYB OLIGO | 20 | 60.00 | 55.00 | 2.00 | 2.00 | (SEQ ID NO: 8) |
| | | | | | | | acacacacacacacacaccg (SEQ ID |
| | | | | | | | NO: 9) |
| ErbB1 | LEFT PRIMER | 20 | 59.97 | 50.00 | 4.00 | 2.00 | acttgacaggggaaacatgc (SEQ ID |
| | RIGHT PRIMER | 20 | 60.00 | 55.00 | 3.00 | 3.00 | NO: 10) caaggtctgggaaccactgt |
| | HYB OLIGO | 20 | 60.09 | 40.00 | 4.00 | 2.00 | (SEQ ID NO: 11) |
| | | | | | | | ttgcacaattccaaccttga (SEQ ID |
| | | | | | | | NO: 12) |
| ErbB1 | LEFT PRIMER | 20 | 60.00 | 55.00 | 3.00 | 1.00 | ggctcacagcaaacttctcc (SEQ ID |
| | RIGHT PRIMER | 20 | 59.97 | 50.00 | 4.00 | 1.00 | NO: 13) gcatgtttcccctgtcaagt |
| | HYB OLIGO | 20 | 60.00 | 55.00 | 2.00 | 2.00 | (SEQ ID NO: 14) |
| | | | | | | | acacacacacacacacaccg (SEQ ID |
| | | | | | | | NO: 15) |
| ErbB1 | LEFT PRIMER | 20 | 60.00 | 55.00 | 3.00 | 1.00 | gggctcacagcaaacttctc (SEQ ID |
| | RIGHT PRIMER | 20 | 59.97 | 50.00 | 4.00 | 1.00 | NO: 16) gcatgtttcccctgtcaagt |
| | HYB OLIGO | 20 | 60.00 | 55.00 | 2.00 | 2.00 | (SEQ ID NO: 17) |
| | | | | | | | acacacacacacacacaccg (SEQ ID |
| | | | | | | | NO: 18) |
| ErbB2/ | LEFT PRIMER | 20 | 59.99 | 55.00 | 2.00 | 0.00 | ccataacacccacctctgct (SEQ ID |
| HER2 | RIGHT PRIMER | 20 | 59.95 | 55.00 | 6.00 | 3.00 | NO: 19) actggctgcagttgacacac |
| | HYB OLIGO | 20 | 60.06 | 55.00 | 4.00 | 1.00 | (SEQ ID NO: 20) |
| ErbB2/ | LEFT PRIMER | 20 | 59.94 | 55.00 | 8.00 | 0.00 | acacagcggtgtgagaagtg (SEQ ID |
| HER2 | RIGHT PRIMER | 20 | 60.09 | 65.00 | 4.00 | 0.00 | NO: 22) aggccaggggagag (SEQ |
| | HYB OLIGO | 20 | 59.65 | 55.00 | 3.00 | 3.00 | ID NO: 23) tcagaccctcttgggaccta |
| | | | | | | | (SEQ ID NO: 24) |
| ErbB2/ | LEFT PRIMER | 20 | 60.16 | 55.00 | 3.00 | 3.00 | gcctccacttcaaccacagt (SEQ ID |
| HER2 | RIGHT PRIMER | 20 | 59.99 | 55.00 | 4.00 | 2.00 | NO: 25) cccacgtccgaaaggta |
| | HYB OLIGO | 20 | 60.31 | 55.00 | 5.00 | 2.00 | (SEQ ID NO: 26) |
| | | | | | | | tgtgactgcctgtccctaca (SEQ ID |
| | | | | | | | NO: 27) |
| ErbB2/ | LEFT PRIMER | 20 | 59.84 | 55.00 | 4.00 | 0.00 | cccagctctttgaggacaac (SEQ ID |
| HER2 | RIGHT PRIMER | 20 | 59.91 | 50.00 | 8.00 | 0.00 | NO: 28) agccagctggttgttcttgt |
| | HYB OLIGO | 20 | 59.89 | 55.00 | 10.00 | 3.00 | (SEQ ID NO: 29) |
| | | | | | | | agcttcgaagcctcacagag (SEQ ID |
| | | | | | | | NO: 30) |
| ErbB2/ | LEFT PRIMER | 20 | 59.91 | 55.00 | 4.00 | 3.00 | tggggagagagttctgagga (SEQ ID |
| HER2 | RIGHT PRIMER | 20 | 60.16 | 50.00 | 7.00 | 1.00 | NO: 31) acagatgccactgtggttga |
| | HYB OLIGO | 20 | 60.16 | 57.89 | 8.00 | 8.00 | (SEQ ID NO: 32) |
| | | | | | | | gactgctgccatgagcagt (SEQ ID |
| | | | | | | | NO: 33) |
| ErbB2/ | LEFT PRIMER | 20 | 59.84 | 55.00 | 4.00 | 0.00 | cccagctctttgaggacaac (SEQ ID |
| HER2 | RIGHT PRIMER | 20 | 59.87 | 55.00 | 4.00 | 0.00 | NO: 34) ggatcaagacccctcctttc |
| | HYB OLIGO | 20 | 59.89 | 55.00 | 10.00 | 3.00 | (SEQ ID NO: 35) |
| | | | | | | | agcttcgaagcctcacagag (SEQ ID |
| | | | | | | | NO: 36) |
| ErbB2/ | LEFT PRIMER | 20 | 59.99 | 55.00 | 2.00 | 0.00 | ccataacacccacctctgct (SEQ ID |
| HER2 | RIGHT PRIMER | 20 | 59.95 | 55.00 | 6.00 | 3.00 | NO: 37) actggctgcagttgacacac |
| | HYB OLIGO | 20 | 60.06 | 55.00 | 4.00 | 1.00 | (SEQ ID NO: 38) |
| | | | | | | | accaagctctgctccacact (SEQ ID |
| | | | | | | | NO: 39) |
| ErbB2/ | LEFT PRIMER | 20 | 59.93 | 50.00 | 5.00 | 3.00 | ccatctgcaccattgatgtc (SEQ ID NO: 40) |
| HER2 | RIGHT PRIMER | 20 | 60.02 | 60.00 | 3.00 | 1.00 | gagcggaaggtgctgtc (SEQ ID |
| | HYB OLIGO | 20 | 59.97 | 50.00 | 4.00 | 4.00 | NO: 41) cgggagttggtgtctgaatt |
| | | | | | | | (SEQ ID NO: 42) |
| ErbB2/ | LEFT PRIMER | 20 | 60.05 | 55.00 | 2.00 | 0.00 | ccctcatccaccataacacc (SEQ ID |
| HER2 | RIGHT PRIMER | 20 | 59.95 | 55.00 | 6.00 | 3.00 | NO: 43) actggctgcagttgacacac |
| | HYB OLIGO | 20 | 60.06 | 55.00 | 4.00 | 1.00 | (SEQ ID NO: 44) |
| | | | | | | | accaagctctgctccacact (SEQ ID |
| | | | | | | | NO: 45) |
| ErbB2/ | LEFT PRIMER | 20 | 60.05 | 50.00 | 3.00 | 2.00 | cgcttttggcacagtctaca (SEQ ID |
| HER2 | RIGHT PRIMER | 20 | 60.07 | 55.00 | 5.00 | 3.00 | NO: 46) tcccggacatggtctaagag |
| | HYB OLIGO | 20 | 59.93 | 45.00 | 6.00 | 2.00 | (SEQ ID NO: 47) |
| | | | | | | | aattccagtggccatcaaag (SEQ ID |
| | | | | | | | NO: 48) |
| ErbB2/ | LEFT PRIMER | 20 | 59.93 | 45.00 | 6.00 | 2.00 | aattccagtggccatcaaag (SEQ ID |
| HER2 | RIGHT PRIMER | 20 | 60.07 | 55.00 | 5.00 | 3.00 | NO: 49) tcccggacatggtctaagag |
| | HYB OLIGO | 20 | 60.14 | 55.00 | 5.00 | 2.00 | (SEQ ID NO: 50) |
| | | | | | | | ggtgacacagcttatgccct (SEQ ID |
| | | | | | | | NO: 51) |
| ErbB2/ | LEFT PRIMER | 20 | 59.99 | 55.00 | 2.00 | 0.00 | ccataacacccacctctgct (SEQ ID |
| HER2 | RIGHT PRIMER | 20 | 59.95 | 55.00 | 6.00 | 3.00 | NO: 52) actggctgcagttgacacac |
| | HYB OLIGO | 20 | 60.06 | 55.00 | 4.00 | 1.00 | (SEQ ID NO: 53) |
| | | | | | | | accaagctctgctccacact (SEQ ID |
| | | | | | | | NO: 54) |
| ErbB2/ | LEFT PRIMER | 20 | 60.05 | 55.00 | 2.00 | 0.00 | ccctcatccaccataacacc (SEQ ID |
| HER2 | RIGHT PRIMER | 20 | 59.95 | 55.00 | 6.00 | 3.00 | NO: 55) actggctgcagttgacacac |
| | HYB OLIGO | 20 | 60.06 | 55.00 | 4.00 | 1.00 | (SEQ ID NO: 56) |
| | | | | | | | accaagctctgctccacact (SEQ ID |
| | | | | | | | NO: 57) |
| ErbB2/ | LEFT PRIMER | 20 | 60.05 | 50.00 | 3.00 | 2.00 | cgcttttggcacagtctaca (SEQ ID |
| HER2 | RIGHT PRIMER | 20 | 60.07 | 55.00 | 5.00 | 3.00 | NO: 58) tcccggacatggtctaagag |
| | HYB OLIGO | 20 | 59.93 | 45.00 | 6.00 | 2.00 | (SEQ ID NO: 59) |
| | | | | | | | aattccagtggccatcaaag (SEQ ID |
| | | | | | | | NO: 60) |
| ErbB2/ | LEFT PRIMER | 20 | 59.93 | 45.00 | 6.00 | 2.00 | aattccagtggccatcaaag (SEQ ID |
| HER2 | RIGHT PRIMER | 20 | 60.07 | 55.00 | 5.00 | 3.00 | NO: 61) tcccggacatggtctaagag |
| | HYB OLIGO | 20 | 60.14 | 55.00 | 5.00 | 2.00 | (SEQ ID NO: 62) |
| | | | | | | | ggtgacacagcttatgccct (SEQ ID |
| | | | | | | | NO: 63) |
| ErbB2/ | LEFT PRIMER | 20 | 59.93 | 45.00 | 6.00 | 2.00 | aattccagtggccatcaaag (SEQ ID |
| HER2 | RIGHT PRIMER | 20 | 59.93 | 50.00 | 5.00 | 3.00 | NO: 64) tttcccggacatggtctaag |
| | HYB OLIGO | 20 | 60.14 | 55.00 | 5.00 | 2.00 | (SEQ ID NO: 65) |
| | | | | | | | ggtgacacagcttatgccct (SEQ ID |
| | | | | | | | NO: 66) |
| ErbB3 | LEFT PRIMER | 20 | 59.95 | 60.00 | 3.00 | 3.00 | gagcccagaggagaagact (SEQ ID |
| | RIGHT PRIMER | 20 | 59.99 | 55.00 | 6.00 | 0.00 | NO: 67) tctgatgcgacagacactcc |
| | HYB OLIGO | 20 | 59.83 | 60.00 | 3.00 | 0.00 | (SEQ ID NO: 68) |
| | | | | | | | gagtctgagtgttcggaggg (SEQ ID |
| | | | | | | | NO: 69) |
| ErbB3 | LEFT PRIMER | 20 | 59.93 | 50.00 | 4.00 | 2.00 | aattgactggagggacatcg (SEQ ID |
| | RIGHT PRIMER | 20 | 60.12 | 55.00 | 3.00 | 3.00 | NO: 70) ggagcacagatggtcttggt |
| | HYB OLIGO | 20 | 59.87 | 50.00 | 4.00 | 2.00 | (SEQ ID NO: 71) |
| | | | | | | | aggacaatggcagaagctgt (SEQ ID |
| | | | | | | | NO: 72) |
| ErbB3 | LEFT PRIMER | 20 | 59.93 | 50.00 | 4.00 | 2.00 | aattgactggagggacatcg (SEQ ID |
| | RIGHT PRIMER | 20 | 60.26 | 55.00 | 3.00 | 1.00 | NO: 73) aggagcacagatggtcttgg |
| | HYB OLIGO | 20 | 59.87 | 50.00 | 4.00 | 2.00 | (SEQ ID NO: 74) |
| | | | | | | | aggacaatggcagaagctgt (SEQ ID |
| | | | | | | | NO: 75) |
| ErbB3 | LEFT PRIMER | 20 | 59.87 | 50.00 | 4.00 | 2.00 | aggacaatggcagaagctgt (SEQ ID |
| | RIGHT PRIMER | 20 | 60.32 | 60.00 | 4.00 | 1.00 | NO: 76) cgaggtacacaggctccact |
| | HYB OLIGO | 20 | 60.12 | 55.00 | 3.00 | 2.00 | (SEQ ID NO: 77) |
| | | | | | | | accaagaccatctgtgctcc (SEQ ID |
| | | | | | | | NO: 78) |
| ErbB3 | LEFT PRIMER | 20 | 59.68 | 50.00 | 8.00 | 2.00 | ggaagtttgccatcttcgtc (SEQ ID |
| | RIGHT PRIMER | 20 | 59.87 | 50.00 | 4.00 | 0.00 | NO: 79) acagcttctgccattgtcct |
| | HYB OLIGO | 20 | 59.93 | 50.00 | 4.00 | 2.00 | (SEQ ID NO: 80) |
| | | | | | | | aattgactggagggacatcg (SEQ ID |
| | | | | | | | NO: 81) |
| ErbB3 | LEFT PRIMER | 20 | 60.34 | 60.00 | 6.00 | 2.00 | gagggacccaggtctacgat (SEQ ID |
| | RIGHT PRIMER | 20 | 59.87 | 50.00 | 4.00 | 0.00 | NO: 82) acagcttctgccattgtcct |
| | HYB OLIGO | 20 | 59.93 | 50.00 | 4.00 | 2.00 | (SEQ ID NO: 83) |
| | | | | | | | aattgactggagggacatcg (SEQ ID |
| | | | | | | | NO: 84) |
| ErbB4 | LEFT PRIMER | 20 | 60.04 | 45.00 | 3.00 | 0.00 | tttcgggagtttgagaatgg (SEQ ID |
| | RIGHT PRIMER | 20 | 59.97 | 50.00 | 7.00 | 2.00 | NO: 85) gaaactgtttgccccctgta |
| | HYB OLIGO | 20 | 60.04 | 50.00 | 4.00 | 4.00 | (SEQ ID NO: 86) |
| | | | | | | | aagatggaagatggcctcct (SEQ ID |
| | | | | | | | NO: 87) |
| ErbB4 | LEFT PRIMER | 20 | 60.04 | 45.00 | 3.00 | 0.00 | tttcgggagtttgagaatgg (SEQ ID |
| | RIGHT PRIMER | 20 | 59.97 | 50.00 | 7.00 | 2.00 | NO: 88) gaaactgtttgccccctgta |
| | HYB OLIGO | 20 | 60.04 | 50.00 | 4.00 | 4.00 | (SEQ ID NO: 89) |
| | | | | | | | aagatggaagatggcctcct (SEQ ID |
| | | | | | | | NO: 90) |
| ErbB4 | LEFT PRIMER | 20 | 59.91 | 45.00 | 5.00 | 2.00 | ggtgaatttcgggagtttga (SEQ ID |
| | RIGHT PRIMER | 20 | 59.97 | 50.00 | 7.00 | 2.00 | NO: 91) gaaactgtttgccccctgta |
| | HYB OLIGO | 20 | 60.04 | 50.00 | 4.00 | 4.00 | (SEQ ID NO: 92) |
| | | | | | | | aagatggaagatggcctcct (SEQ ID |
| | | | | | | | NO: 93) |
| ErbB4 | LEFT PRIMER | 20 | 59.97 | 45.00 | 5.00 | 2.00 | ggtgcttttggaacggttta (SEQ ID |
| | RIGHT PRIMER | 20 | 59.84 | 55.00 | 4.00 | 0.00 | NO: 94) aaccggacgtgtggatg |
| | HYB OLIGO | 20 | 59.69 | 45.00 | 4.00 | 3.00 | (SEQ ID NO: 95) |
| | | | | | | | caaggcaaatgtggagttca (SEQ ID |
| | | | | | | | NO: 96) |
| ErbB4 | LEFT PRIMER | 20 | 59.97 | 45.00 | 5.00 | 2.00 | ggtgcttttggaacggttta (SEQ ID |
| | RIGHT PRIMER | 20 | 59.84 | 55.00 | 4.00 | 2.00 | NO: 97) caaccggacgtgtggat |
| | HYB OLIGO | 20 | 59.69 | 45.00 | 4.00 | 3.00 | (SEQ ID NO: 98) |
| | | | | | | | caaggcaaatgtggagttca (SEQ ID |
| | | | | | | | NO: 99) |
| ErbB4 | LEFT PRIMER | 20 | 60.15 | 55.00 | 7.00 | 2.00 | ccagaccaatgtctgtcgtg (SEQ ID |
| | RIGHT PRIMER | 20 | 60.04 | 50.00 | 4.00 | 0.00 | NO: 100) aggaggccatcttccatctt |
| | HYB OLIGO | 20 | 60.04 | 45.00 | 3.00 | 0.00 | (SEQ ID NO: 101) |
| | | | | | | | tttcgggagtttgagaatgg (SEQ ID |
| | | | | | | | NO: 102) |

The AVEC antibody domains had three sources. The antibodies were designed and manufactured as human antibodies according to the methods described [21, 22]. Alternatively they were synthesized, they were manufactured as biosimilars of the antibodies approved by the FDA: trastuzumab, pertuzumab, cetuximab, panitumumab, alemtuzumab, rituximab based upon importing their sequences from IMGT and expressing in human cells. Finally, the antibodies approved by the FDA were incorporated into AVEC: trastuzumab, pertuzumab, cetuximab, panitumumab, alemtuzumab, rituximab. For designing of the DNA plasmid constructs for the aforementioned biosimilar antibodies, the coding sequences were imported from the ImMunoGeneTics antibody sequences' bank (IMGT, Paris, F, EU) [23, 24].

For generating of anti-HBsAg, anti-HBcAg, anti-HBeAg antibodies the B cells were acquired from the patients suffering from the Acute and/or Chronic Hepatitis B. The protocol was identical to that published. HBV isolated from the patients' blood by PEG gradients precipitation or from liver biopsies by CsCl gradient centrifugations, were rapidly frozen, lyophilized and stored [25].

HBsAg were produced in human hepatoma cells transfected with plasmid DNA. Prior to selection, during in vitro evolution, they were reconstituted with buffer and served as the molecular baits. They also served as the controls for anti-HBsAg antibodies [26-32].

For generating of anti-HSVgB and anti-HSVgD antibodies the B cell were acquired from the patients suffering from infections with the Herpes Simplex Viruses. The protocol was identical to that described above.

For generating of anti-HPV-VP 16 and 18 antibodies the B cell were acquired from the patients suffering from the infections with Human Papilloma Viruses. The protocol was identical to that described above.

The above aforementioned original and biosimilar antibodies IgG, scFv, dsFvs, sdFvs, and CDRs were expressed from the DNA constructs assembled through the amplicons driven by the CDR primers listed below [31,32]. The primers were selected after analysis of sequences data base. They were expressed from combinatorial libraries using HEK293 cell and mRNA displays according to published protocols [21, 22, 31, 32]

Primers including, but not limiting to the ones outlined below:

Primers for CDR1:
H1-Forward:
(SEQ ID NO: 103)
5′-GAG GAG GAG GAG GAG GAG GCG GGG CCC AGG CGG
CCC AGG TGC AGC TGG TGC-3′;
H1-Reverse:
(SEQ ID NO: 104)
5′-GCG GAC CCA GCT CAT TTC ATA AKM AKM GAA AKM
GAA AKM AGA GGC TGC ACA GGA GAG-3′
Primers for CDR2:
H2-Forward1:
(SEQ ID NO: 105)
5′-GAA ATG AGC TGG GTC CGC CAG GCT CCA GGA CAA
SGS CTT GAG TGG-3′;
H2-Forward2:
(SEQ ID NO: 106)
5′-GAA ATG AGC TGG GTC CGC CAG GCT CCA GGG AAG
GCC CTG GAG TGG-3′;
H2-Forward3:
(SEQ ID NO: 107)
5′-GAA ATG AGC TGG GTC CGC CAG GCT CCA GGG AAG
GGN CTR GAG TGG-3′;
H2-Reverse1:
(SEQ ID NO: 108)
5′-ATT GTC TCT GGA GAT GGT GAC CCT KYC CTG RAA
CTY3′;
H2-Reverse2:
(SEQ ID NO: 109)
5′-ATT GTC TCT GGA GAT GGT GAA TCG GCC CTT CAC
NGA-3′;
H2-Reverse3:
(SEQ ID NO: 110)
5′-ATT GTC TCT GGA GAT GGT GAC TMG ACT CTT GAG
GGA-3′;
H2-Reverse4:
(SEQ ID NO: 111)
5′-ATT GTC TCT GGA GAT GGT GAC STG GCC TTG GAA
GGA-3′;
H2-Reverse5:
(SEQ ID NO: 112)
5′-ATT GTC TCT GGA GAT GGT AAA CCG TCC TGT GAA
GCC3′;
Primers for CDR3:
H3-Forward1:
(SEQ ID NO: 113)
5′-ACC CTG AGA GCC GAG GAC ACR GCY TTR TAT TAC
TGT3′;
H3-Forward2:
(SEQ ID NO: 114)
5′-ACC CTG AGA GCC GAG GAC ACA GCC AYR TAT TAC
TGT-3′;
H3-Forward3:
(SEQ ID NO: 115)
5′-ACC CTG AGA GCC GAG GAC ACR GCY GTR TAT TAC
TGT-3′;
H3-Reverse:
(SEQ ID NO: 116)
5′-GTG GCC GGC CTG GCC ACT TGA GGA GAC GGT GAC
C-3′
Primers for other CDRs
CDR-H1-Forward
(SEQ ID NO: 117)
5′-CTC TGG ATT CAC CTT CRR TTA TKM TAT GAG CTG
GGT CCG CCA GGC TCC AG-3′
CDR-H2-Forward
(SEQ ID NO: 118)
5′-GGG CTG GAG TGG GTC TCA KBG ATC TMT YMT RRT
RRT RGT ART AHA TAT TAC GCT GAT TCT GTA AAA GGT
CGG TTC ACC ATC TCC AGA G-3′
CDR-H3-9-Reverse
(SEQ ID NO: 119)
5′-CTG GCC CCA GTA GTC GAA MNN MNN MNN MNN TYT
CGC ACA GTA ATA CAC GGC-3′
CDR-H3-14-Reverse
(SEQ ID NO: 120)
5′-CTG GCC CCA GTA GTC GAA MNN MNN MNN MNN MNN
MNN MNN AVS AYC TYT CGC ACA GTA ATA CAC GGC-3′
CDR-H3-20-Reverse
(SEQ ID NO: 121)
5′-CTG GCC CCA GAC GTC CAT ASC ATH AKM AKA AKA
MNN MNN MNN MNN MNN MNN MNN AMB AVB ANV TYT CGC
ACA GTA ATA CAC GGC-3′
CDR-H3-2055-Reverse
(SEQ ID NO: 122)
5′-CTG GCC CCA GAC GTC CAT ASC ATH AKM AKA AKA
ACA MNN MNN MNN MNN ACA MNN AMB AVB ANC TYT CGC
ACA GTA ATA CAC GGC-3′
CDR-L1-Forward
(SEQ ID NO: 123)
5′-GAG GGT CAC CAT CTC TTG TAS TGG CTC TTC ATC
TAA TAT TGG CAR TAA TDM TGT CWM CTG GTA CCA GCA
GCT CCC AG-3′
CDR-L2-Forward
(SEQ ID NO: 124)
5′-CCC AAA CTC CTC ATC TAT KMT RAT ART MAK CGG
CCA AGC GGG GTC CCT GAC CGA TTC-3′
CDR-L3-Reverse
(SEQ ID NO: 125)
5′-GAG GCT GAT TAT TAC TGT GST DCT TGG GAT KMT
AGC CTG ART GST TAT GTC TTC GGC GGA GGC-3′
Primers for FRs
FR3-Forward:
(SEQ ID NO: 126)
5′-ACC ATC TCC AGA GAC AAT TCC-3′
FR3-Reverse:
(SEQ ID NO: 127)
5′-GTC CTC GGC TCT CAG GGT G-3′
FR-H1-Forward:
(SEQ ID NO: 128)
5′-GAG GTG CAG CTG TTG GAG TCT GGG GGA GGC TTG
GTA CAG CCT GGG GGG TCC CTG-3′
FR-H1-Reverse:
(SEQ ID NO: 129)
5′-GCT AAA GGT GAA TCC AGA GGC TGC ACA GGA GAG
TCT CAG GGA CCC CCC AGG CTG-3′
FR-H2-Reverse:
(SEQ ID NO: 130)
5′-TGA GAC CCA CTC CAG CCC CTT CCC TGG AGC CTG GCG
GAC CCA-3′
FR-H3-Reverse:
(SEQ ID NO: 131)
5′-GGC TGT TCA TTT GCA GAT ACA GCG TGT TCT TGG AAT
TGT CTC TGG AGA TGG TGA ACC G-3′
FR-H3-Forward:
(SEQ ID NO: 132)
5′-GTA TCT GCA AAT GAA CAG CCT GAG AGC CGA GGA CAC
GGC CGT GTA TTA CTG TGC G-3′
JH-15-Forward:
(SEQ ID NO: 133)
5′-T-TCG ACT ACT GGG GCC AGG GTA CAC TGG TCA CCG
TGA GCT CA-3′
JH-6-Forward:
(SEQ ID NO: 134)
5′-ATG GAC TGC TGG GGC CAG GGT ACA CTG GTC ACC GTG
AGC TCA-3′
FR-L1-Forward:
(SEQ ID NO: 135)
5′-CAG TCT GTG CTG ACT CAG CCA CCC TCA GCG TCT GGG
ACC CCC-3′
FR-L1 Reverse:
(SEQ ID NO: 136)
5′-ACA AGA GAT GGT GAC CCT CTG CCC GGG GGT CCC AGA
CGC TGA G-3′
FR-L2-Reverse:
(SEQ ID NO: 137)
5′-ATA GAT GAG GAG TTT GGG GGC CGT TCC TGG GAG CTG
CTG GTA CCA G-3′
FR-L3-Reverse:
(SEQ ID NO: 138)
5′-GAT GGC CAG GGA GGC TGA GGT GCC AGA CTT GGA GCC
AGA GAA TCG GTC AGG GAC CCC-3′
FR-L3-F
(SEQ ID NO: 139)
5′-TCA GCC TCC CTG GCC ATC AGT GGG CTC CGG TCC GAG
GAT GAG GCT GAT TAT TAC TGT G-3′
JL-Forward:
(SEQ ID NO: 140)
5′-TAT GTC TTC GGC GGA GGC ACC AAG CTG ACG GTC CTA
GGC-3′
FRH3-short-Reverse:
(SEQ ID NO: 141)
5′-CGC ACA GTA ATA CAC GGC C-3′
JH15-short-Forward:
(SEQ ID NO: 142)
5′-TTC GAC TAC TGG GGC CAG-3′
JH6-short-Forward:
(SEQ ID NO: 143)
5′-ATG GAC GTC TGG GGC CAG GGT ACA CTG-3′
pC3X-Forward:
(SEQ ID NO: 144)
5′-GCA CGA CAG GTT TCC CGA C-3′
pC3X-Reverse:
(SEQ ID NO: 145)
5′-AAC CAT CGA CAG CAC CG-3′
H1-Reverse:
(SEQ ID NO: 146)
5′-GCT AAA GGT GAA TCC AGA G-3′
H2-Forward:
(SEQ ID NO: 147)
5′-CTG GGT CCG CCA GGC TCC AG-3′
H2-Reverse:
(SEQ ID NO: 148)
5′-TGA GAC CCA CTC CAG CCC-3′
H3-Forward:
(SEQ ID NO: 149)
5′-CGG TTC ACC ATC TCC AGA G-3′
L1-Reverse:
(SEQ ID NO: 150)
5′-CAA GAG ATG GTG ACC CTC-3′
L2-Forward:
(SEQ ID NO: 151)
5′-CTG GTA CCA GCA GCT CCC AG-3′
L2-Reverse:
(SEQ ID NO: 152)
5′-ATA GAT GAG GAG TTT GGG-3′
L3-Forward:
(SEQ ID NO: 153)
5′-GGG GTC CCT GAC CGA TTC-3′
L3-Reverse:
(SEQ ID NO: 154)
5′-CAC AGT AAT AAT CAG CCT C-3′
JL-short-Forward:
(SEQ ID NO: 155)
5′-TAT GTC TTC GGC GGA GGC-3′

The original generated antibodies and/or their fragments, which were developed according to the procedures outlined above and in Examples, while relying upon the aforementioned CDRs and tested with the recombinant receptors outlined above, were incorporated as the AVEC antibody domains. The sequences including, but not limited to those, which are listed below.

TABLE
Sequences of CDRs including, but not limiting to the ones outlined below.

| Receptor | | Exemplar Sequence (nucleic | Translation (amino |
|---|---|---|---|
| Target | Consensus Sequence (5′→3′) | acid sequence) (5′→3′) | acid sequence) (5′→3′) |
| anti-huEGFR H1ₐ | ggnttywsnttywsnacntayggnatgcaytrr | ggcttcttcacctatggcatgcatta | GFSFSTYGMH |
| | (SEQ ID NO: 156) | a (SEQ ID NO: 210) | (SEQ ID NO: 264) |
| anti-huEGFR H2ₐ | gtnathtgggaygayggnwsntayaartayttyg | gtgatttgggatgatggcagctataaatattttgg | VIWDDGSYKYFGD |
| | gngaywsngtntrr (SEQ ID NO: 157) | c gacgtgtaa (SEQ ID NO: 211) | S V (SEQ ID |
| | | | NO: 265) |
| anti-huEGFR H3ₐ | gtnathtgggaygayggnwsntayaartayttyg | gtgatttgggatgatggcagctataaatattttgg | DAITMVRGVMKEYF |
| | gngaywsngtntrr (SEQ ID NO: 158) | c gacgtgtaa (SEQ ID NO: 212) | DY (SEQ ID |
| | | | NO: 266) |
| anti-huEGFR H1_{b} | ggnttyacntaywsnacntayggnatgcaytrr | ggctttacctacacctatggcatgcattaa | GFTYSTYGMH |
| | (SEQ ID NO: 159) | (SEQ ID NO: 213) | (SEQ ID NO: 267) |
| anti-huEGFR H2_{b} | gtnathtgggargayggnwsntayaartaytay | gtgatttgggaagatggcagctataaatattatg | VIWEDGSYKYYGD |
| | ggngaywsngtntrr (SEQ ID NO: 160) | g cgacgtgtaa (SEQ ID NO: 214) | S V (SEQ ID |
| | | | NO: 268) |
| anti-huEGFR H3_{b} | gayggnathwsnatggtnmgngcngtnatg | gatggcatcatggtgcgcgcggtgatgcgcgatt | DGISMVRAVMRDY |
| | m gngaytayttygayttytrr | attttgatttttaa (SEQ ID NO: 215) | F DF (SEQ ID |
| | (SEQ ID NO: 161) | | NO: 269) |
| anti-huEGFR H1_{c} | ggnttyacnttywsnacnttygcnatgcaytrr | ggctttaccttcacctttgcgatgcattaa | GFTFSTFAMH |
| | (SEQ ID NO: 162) | (SEQ ID NO: 216) | (SEQ ID NO: 270) |
| anti-huEGFR H2_{c} | gtnathtgggaygayggnwsntayaarttytayg | gtgatttgggatgatggcagctataaattttatgc | VIWDDGSYKFYAE |
| | cngarwsngtntrr (SEQ ID NO: 163) | g gaaagcgtgtaa (SEQ ID NO: 217) | S V (SEQ ID |
| | | | NO: 271) |
| anti-huEGFR H3_{c} | gayggnathacnatggtnmgnggngtnatg | gatggcattaccatggtgcgcggcgtgatgcgcg | DGITMVRGVMRDYF |
| | m gngaytayttygayttytrr | attattttgatttttaa (SEQ ID NO: 218) | DF (SEQ ID NO: 272) |
| | (SEQ ID NO: 164) | | |
| anti-huEGFR L1ₐ | mgngcnwsncargayathwsnwsngcnytn | cgcgcgagccaggatatcagcgcgctggtgtaa | RASQDISSALV |
| | gtntrr (SEQ ID NO: 165) | (SEQ ID NO: 219) | (SEQ ID NO: 273) |
| anti-huEGFR L2ₐ | gaygcnwsnwsnytngartrr | gatgcgagcagcctggaata | DASSLE |
| | (SEQ ID NO: 166) | a (SEQ ID NO: 220) | (SEQ ID NO: 274) |
| anti-huEGFR L3ₐ | carcarttyaaywsntayccnytnacntr | cagcagtttaacagctatccgctgaccta | QQFNSYPLT |
| | r (SEQ ID NO: 167) | a (SEQ ID NO: 221) | (SEQ ID NO: 275) |
| anti-huEGFR L1_{b} | mgngcnwsncargarathwsnwsngcnytny | cgcgcgagccaggaaatcagcgcgctgctgta | RASQEISSALL |
| | tntrr (SEQ ID NO: 168) | a (SEQ ID NO: 222) | (SEQ ID NO: 276) |
| anti-huEGFR L2_{b} | gargcnwsnwsnytngaracntrr | gaagcgagcagcctggaaaccta | EASSLET |
| | (SEQ ID NO: 169) | a (SEQ ID NO: 223) | (SEQ ID NO: 277) |
| anti-huEGFR L3_{b} | caraayttyaaywsntayccnytnwsntr | cagaactttaacagctatccgctgagcta | QNFNSYPLS |
| | r (SEQ ID NO: 170) | a (SEQ ID NO: 224) | (SEQ ID NO: 278) |
| anti-huEGFR L1_{c} | mgngcnwsncargayathacnwsngcnytny | cgcgcgagccaggatattaccagcgcgctgctgt | RASQDITSALL |
| | tntrr (SEQ ID NO: 171) | aa (SEQ ID NO: 225) | (SEQ ID NO: 279) |
| anti-huEGFR L2_{c} | gaygcnwsnwsnytngarwsn | gatgcgagcagcctggaaag | DASSLES |
| | (SEQ ID NO: 172) | c (SEQ ID NO: 226) | (SEQ ID NO: 280) |
| anti-huEGFR L3_{c} | aaycarttycarwsntayccnytnwsn | aaccagtttcagagctatccgctgag | NQFQSYPLS |
| | (SEQ ID NO: 173) | c (SEQ ID NO: 227) | (SEQ ID NO: 281) |
| anti-huEGFRVIII H1ₐ | ggnttywsnttymgnaarttyggnatgwsntr | ggcttctttcgcaaatttggcatgagcta | GFSFRKFGMS |
| | r (SEQ ID NO: 174) | a (SEQ ID NO: 228) | (SEQ ID NO: 282) |
| anti-huEGFRvIII H2ₐ | wsnathwsnacnggnggntayaaywsntayt | agcatcaccggcggctataacagctattacgata | SISTGGYNSYYSD |
| | aywsngayaaygtntrr (SEQ ID NO: 175) | acgtgtaa (SEQ ID NO: 229) | N V (SEQ ID |
| | | | NO: 283) |
| anti-huEGFRvIII H3ₐ | ggnttywsnwsnacnwsntaygcnatggayta | ggcttcagcaccagctatgcgatggattattaa | GFSSTSYAMDY |
| | ytrr (SEQ ID NO: 176) | (SEQ ID NO: 230) | (SEQ ID NO: 284) |
| anti-huEGFRvIII H1_{b} | ggnttyacnttyaaraarttyggnatgwsntr | ggctttacctttaaaaaatttggcatgagcta | GFTFKKFGMS |
| | r (SEQ ID NO: 177) | a (SEQ ID NO: 231) | (SEQ ID NO: 285) |
| anti-huEGFRvIII H2_{b} | wsnathwsnacnggnggnttyaayacntayt | agcatcaccggcggctttaacacctattacgata | SISTGGFNTYYSDN |
| | a ywsngayaaygtntrr (SEQ ID | acgtgtaa (SEQ ID NO: 232) | V (SEQ ID NO: 286) |
| | NO: 178) | | |
| anti-huEGFRvIII H3_{b} | ggntaywsnwsnacnwsnttyggnatggayta | ggctacagcaccagctttggcatggattattaa | GYSSTSFGMDY |
| | ytrr (SEQ ID NO: 179) | (SEQ ID NO: 233) | (SEQ ID NO: 287) |
| anti-huEGFRvIII H1_{c} | ggntaywsnttymgnaarttyggnatgwsntr | ggctactttcgcaaatttggcatgagcta | GYSFRKFGMS |
| | r (SEQ ID NO: 180) | a (SEQ ID NO: 234) | (SEQ ID NO: 288) |
| anti-huEGFRvIII H2_{c} | wsnathwsnacnggnggntaycaracntayta | agcatcaccggcggctatcagacctattacgata | SISTGGYQTYYSD |
| | ywsngayaaygtntrr (SEQ ID NO: 181) | acgtgtaa (SEQ ID NO: 235) | N V (SEQ ID |
| | | | NO: 289) |
| anti-huEGFRvIII H3_{c} | ggntaywsnwsnacnwsntaygcnatggaytt | ggctacagcaccagctatgcgatggatttttaa | GYSSTSYAMDF |
| | ytrr (SEQ ID NO: 182) | (SEQ ID NO: 236) | (SEQ ID NO: 290) |
| anti-huEGFRvIII L1ₐ | mgngcnwsncarwsngtncaywsngayggn | cgcgcgagccagagcgtgcacgatggcaacac | RASQSVHSDGNTY |
| | aayacntayatgcartrr (SEQ ID NO: 183) | ctatatgcagtaa (SEQ ID NO: 237) | M Q (SEQ ID |
| | | | NO: 291) |
| anti-huEGFRvIII L2ₐ | gcngcnwsnaaymgnttywsntr | gcggcgagcaaccgcttcta | AASNRFS |
| | r (SEQ ID NO: 184) | a (SEQ ID NO: 238) | (SEQ ID NO: 292) |
| anti-huEGFRvIII L3ₐ | carcarggnacncarytnccnmgnacntr | cagcagggcacccagctgccgcgcacctaa | QQGTQLPRT |
| | r (SEQ ID NO: 185) | (SEQ ID NO: 239) | (SEQ ID NO: 293) |
| anti-huEGFRvIII L1_{b} | mgnwsnwsncarwsngtncaywsngaygg | Cgcagcagccagagcgtgcacgatggcaaca | RSSQSVHSDGNSY |
| | naaywsntayytnwsntrr | gctatctgagctaa (SEQ ID NO: 240) | L S (SEQ ID |
| | (SEQ ID NO: 186) | | NO: 294) |
| anti-huEGFRvIII L2_{b} | ggngcnwsnaayaarttywsntrr | ggcgcgagcaacaaattcta | GASNKFS |
| | (SEQ ID NO: 187) | a (SEQ ID NO: 241) | (SEQ ID NO: 295) |
| anti-huEGFRvIII L3_{b} | carcarggnacncarytnccnmgnacntr | Cagcagggcacccagctgccgcgcacctaa | QQGTQLPRT |
| | r (SEQ ID NO: 188) | (SEQ ID NO: 242) | (SEQ ID NO: 296) |
| anti-huEGFRvIII L1_{c} | aarwsncarwsnytngtncaywsngayggna | aaaagccagagcctggtgcacgatggcaaca | KSQSLVHSDGNSY |
| | aywsntayytnwsntrr (SEQ ID NO: 189) | g ctatctgagctaa (SEQ ID NO: 243) | L S (SEQ ID |
| | | | NO: 297) |
| anti-huEGFRvIII L2_{c} | mgnathwsnaaymgnttywsntr | cgcatcaaccgcttctaa (SEQ ID NO: 244) | RISNRFS |
| | r (SEQ ID NO: 190) | | (SEQ ID NO: 298) |
| anti-huEGFRvIII L3_{c} | carcarggnacncarytnccnmgnacntr | cagcagggcacccagctgccgcgcacctaa | QQGTQLPRT |
| | r (SEQ ID NO: 191) | (SEQ ID NO: 245) | (SEQ ID NO: 299) |
| anti-huTfR H1ₐ | ggntaywsntaywsnwsntaytggatgtrr | ggctactacagctattggatgta | GYSYSSYWM |
| | (SEQ ID NO: 192) | a (SEQ ID NO: 246) | (SEQ ID NO: 300) |
| anti-huTfR H2ₐ | gcnathgayccnmgnaaywsngayacnath | gcgattgatccgcgcaacagcgataccatttata | AIDPRNSDTIYNPQ |
| | t ayaayccncarttytrr | acccgcagttttaa (SEQ ID NO: 247) | F (SEQ ID NO: 301) |
| | (SEQ ID NO: 193) | | |
| anti-huTfR H3ₐ | ytntaytaytaygaywsntrr | ctgtattattatgactaa | LYYYDS |
| | (SEQ ID NO: 194) | (SEQ ID NO: 248) | (SEQ ID NO: 302) |
| anti-huTfR H1_{b} | ggntayacnathwsnwsntaytggatgtrr | ggctataccatcagctattggatgta | GYTISSYWM |
| | (SEQ ID NO: 195) | a (SEQ ID NO: 249) | (SEQ ID NO: 303) |
| anti-huTfR H2_{b} | gcngcngayccnmgnaaywsngayacnath | gcggcggatccgcgcaacagcgataccatttatc | AADPRNSDTIYQP |
| | t aycarccncartaytrr | agccgcagtattaa | Q Y (SEQ ID |
| | (SEQ ID NO: 196) | (SEQ ID NO: 250) | NO: 304) |
| anti-huTfR H3_{b} | ytntaytayttygaywsntrr | ctgtattattttgactaa (SEQ ID NO: 251) | LYYFDS |
| | (SEQ ID NO: 197) | | (SEQ ID NO: 305) |
| anti-huTfR H1_{c} | ggntayacngcnacnacntaytggatgtr | ggctataccgcgaccacctattggatgtaa | GYTATTYWM |
| | r (SEQ ID NO: 198) | (SEQ ID NO: 252) | (SEQ ID NO: 306) |
| anti-huTfR H2_{c} | atgathcayccnwsngaywsngargtnmgnyt | atgattcatccgagcgacgaagtgcgcctgaac | MIHPSDSEVRLN |
| | naaycartrr | cagtaa | Q (SEQ ID |
| | (SEQ ID NO: 199) | SEQ ID NO: 253) | NO: 307) |
| anti-huTfR H3_{c} | ytntaytayttygarwsntrr | ctgtattattttgaaagcta | LYYFES |
| | (SEQ ID NO: 200) | a (SEQ ID NO: 254) | (SEQ ID NO: 308) |
| anti-huTfR L1ₐ | gayathaayaaytaygtntgytrr | gatattaacaactatgtgtgcta | DINNYVC |
| | (SEQ ID NO: 201) | a (SEQ ID NO: 255) | (SEQ ID NO: 309) |
| anti-huTfR L2ₐ | aargcnaaymgnytngtngaytrr | aaagcgaaccgcctggtggatta | KANRLVD |
| | (SEQ ID NO: 202) | a (SEQ ID NO: 256) | (SEQ ID NO: 310) |
| anti-huTfR L3ₐ | ytncartaygaygarttyccntayacntr | ctgcagtatgatgaatttccgtatacctaa | LQYDEFPYT |
| | r (SEQ ID NO: 203) | (SEQ ID NO: 257) | (SEQ ID NO: 311) |
| anti-huTfR L1_{b} | garathaayaaytayytntgytrr | gaaattaacaactatctgtgcta | EINNYLC |
| | (SEQ ID NO: 204) | a (SEQ ID NO: 258) | (SEQ ID NO: 312) |
| anti-huTfR L2_{b} | mgngcnaayaarytngtngaytr | cgcgcgaacaaactggtggatta | RANKLVD |
| | r (SEQ ID NO: 205) | a (SEQ ID NO: 259) | (SEQ ID NO: 313) |
| anti-huTfR L3_{b} | ytncartaygaygayttyccntayacntr | ctgcagtatgatgattttccgtataccta | LQYDDFPYT |
| | r (SEQ ID NO: 206) | a (SEQ ID NO: 260) | (SEQ ID NO: 314) |
| anti-huTfR L1c | gayathaaycarttyytntgytrr | gatattaaccagtttctgtgcta | DINQFLC |
| | (SEQ ID NO: 207) | a (SEQ ID NO: 261) | (SEQ ID NO: 315) |
| anti-huTfR L2_{c} | mgngcnaaymgnytngtngaytr | cgcgcgaaccgcctggtggatta | RANRLVD |
| | r (SEQ ID NO: 208) | a (SEQ ID NO: 262) | (SEQ ID NO: 316) |
| anti-huTfR L3_{c} | gtncartaygaygarttyccntaywsntr | gtgcagtatgatgaatttccgtacta | VQYDEFPYS |
| | r (SEQ ID NO: 209) | a (SEQ ID NO: 263) | (SEQ ID NO: 317) |

*The consensus sequences are degeneracy sequences which follow the standard IUPAC symbols for DNA (R = A or G; Y =C or T; M = A or C; W = A or T; S = C or G; B = C, G or T; D = A, G or T; H = A, C or T; V = A, C or G; and N is any nucleotide (A, C G or T)).

In some embodiments, the biosimilar antibodies and/or their fragments were developed according to the procedures outlined above and in Examples and in References, while relying upon the DNA constructs designed and prepared based upon the sequences imported from the IMGT antibody sequences bank and tested with the recombinant receptors outlined above. They were incorporated as the AVEC antibody domains. Their sequences are listed below.

TABLE
Sequences of biosimilar antibodies′ Fvs used herein including, but not limiting to
the ones outlined below.

Trastuzumab biosimilar-anti-HER2
trastuzumab|Humanized||H-GAMMA-1

| | |
|---|---|
| EVQLVESGGGLVQPGGSLRLSCAASGFNIKDTYIHWVRQAPGKGLEWVARIYPTNGYTRY | 60 |
| ADSVKGRFTISADTSKNTAYLQMNSLRAEDTAVYYCSRWGGDGFYAMDYWGQGTLVTVSS | 120 |
| ASTKGPSVFPLAPSSKSTSGGTAALGCLVKDYFPEPVTVSWNSGALTSGVHTFPAVLQSS | 180 |
| GLYSLSSVVTVPSSSLGTQTYICNVNHKPSNTKVDKKVEPKSCDKTHTCPPCPAPELLGG | 240 |
| PSVFLFPPKPKDTLMISRTPEVTCVVVDVSHEDPEVKFNWYVDGVEVHNAKTKPREEQYN | 300 |
| STYRVVSVLTVLHQDWLNGKEYKCKVSNKALPAPIEKTISKAKGQPREPQVYTLPPSREE | 360 |
| MTKNQVSLTCLVKGFYPSDIAVEWESNGQPENNYKTTPPVLDSDGSFFLYSKLTVDKSRW | 420 |
| QQGNVFSCSVMHEALHNHYTQKSLSLSPGK | 450 (SEQ ID NO: 318) |
| trastuzumab\|Humanized\|\|L-KAPPA (V-KAPPA) | |
| DIQMTQSPSSLSASVGDRVTITCRASQDVNTAVAWYQQKPGKAPKLLIYSASFLYSGVPS | 60 |
| RFSGSRSGTDFTLTISSLQPEDFATYYCQQHYTTPPTFGQGTKVEIKRTVAAPSVFIFPP | 120 |
| SDEQLKSGTASVVCLLNNFYPREAKVQWKVDNALQSGNSQESVTEQDSKDSTYSLSSTLT | 180 |
| LSKADYEKHKVYACEVTHQGLSSPVTKSFNRGEC | 214 (SEQ ID NO: 319) |
| Cetuximab biosimilar-anti-EGFR | |
| cetuximab\|Chimeric\|\|H-GAMMA-1 | |
| QVQLKQSGPGLVQPSQSLSITCTVSGFSLTNYGVHWVRQSPGKGLEWLGVIWSGGNTDYN | 60 |
| TPFTSRLSINKDNSKSQVFFKMNSLQSNDTAIYYCARALTYYDYEFAYWGQGTLVTVSAA | 120 |
| STKGPSVFPLAPSSKSTSGGTAALGCLVKDYFPEPVTVSWNSGALTSGVHTFPAVLQSSG | 180 |
| LYSLSSVVTVPSSSLGTQTYICNVNHKPSNTKVDKRVEPKSCDKTHTCPPCPAPELLGGP | 240 |
| SVFLFPPKPKDTLMISRTPEVTCVVVDVSHEDPEVKFNWYVDGVEVHNAKTKPREEQYNS | 300 |
| TYRVVSVLTVLHQDWLNGKEYKCKVSNKALPAPIEKTISKAKGQPREPQVYTLPPSREEM | 360 |
| TKNQVSLTCLVKGFYPSDIAVEWESNGQPENNYKTTPPVLDSDGSFFLYSKLTVDKSRWQ | 420 |
| QGNVFSCSVMHEALHNHYTQKSLSLSPGK | 449 (SEQ ID NO: 320) |
| cetuximab\|Chimeric\|\|L-KAPPA (V-KAPPA) | |
| DILLTQSPVILSVSPGERVSFSCRASQSIGTNIHWYQQRTNGSPRLLIKYASESISGIPS | 60 |
| RFSGSGSGTDFTLSINSVESEDIADYYCQQNNNWPTTFGAGTKLELKRTVAAPSVFIFPP | 120 |
| SDEQLKSGTASVVOLLNNFYPREAKVQWKVDNALQSGNSQESVTEQDSKDSTYSLSSTLT | 180 |
| LSKADYEKHKVYACEVTHQGLSSPVTKSFNRGEC | 214 (SEQ ID NO: 321) |

Pertuzumab biosimilar-anti-HER2
pertuzumab|Humanized||H
EVQLVESGGGLVQPGGSLRLSCAASGFTFTDYTMDWVRQAPGKGLEWVADVNPNSGGSIY
NQRFKGRFTLSVDRSKNTLYLQMNSLRAEDTAVYYCARNLGPSFYFDYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPG (SEQ ID NO: 322)
pertuzumab|Humanized||H
DIQMTQSPSSLSASVGDRVTITCKASQDVSIGVAWYQQKPGKAPKLLIYSASYRYTGVPS
RFSGSGSGTDFTLTISSLQPEDFATYYCQQYYIYPYTFGQGTKVEIKRTVAAPSVFIFPP
SDEQLKSGTASVVOLLNNFYPREAKVQWKVDNALQSGNSQESVTEQDSKDSTYSLSSTLT
LSKADYEKHKVYACEVTHQGLSSPVTKSFNRGEC (SEQ ID NO: 323)
Rituximab biosimilar-anti-CD20
>Rituximab|chimeric|H
QVQLQQPGAELVKPGASVKMSCKASGYTFTSYNMHWVKQTPGRGLEWIGAIYPGNGDTSY
NQKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKAEPKSCDKTHTCPPCPAPE
LLG
GPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSR
D
ELTKNQVSLTCLVKGFYPSDIAVEWESNGQPENNYKTTPPVLDSDGSFFLYSKLTVDKS
RWQQGNVFSCSVMHEALHNHYTQKSLSLSPGK (SEQ ID NO: 324)
>Rituximab ′Chimeric||L
QIVLSQSPAILSASPGEKVTMTCRASSSVSYIHWFQQKPGSSPKPWIYATSNLASGVPVR
FSGSGSGTSYSLTISRVEAEDAATYYCQQWTSNPPTFGGGTKLEIKRTVAAPSVFIFPPS
DEQLKSGTASVVOLLNNFYPREAKVQWKVDNALQSGNSQESVTEQDSKDSTYSLSSTLTL
SKADYEKHKVYACEVTHQGLSSPVTKSFNRGEC (SEQ ID NO: 325)
Alemtuzumab biosimilar-anti-CD52
alemtuzumab|Humanized||H-GAMMA-1

| | |
|---|---|
| QVQLQESGPGLVRPSQTLSLTCTVSGFTFTDFYMNWVRQPPGRGLEWIGFIRDKAKGYTT | 60 |
| EYNPSVKGRVTMLVDTSKNQFSLRLSSVTAADTAVYYCAREGHTAAPFDYWGQGSLVTVS | 120 |
| SASTKGPSVFPLAPSSKSTSGGTAALGCLVKDYFPEPVTVSWNSGALTSGVHTFPAVLQS | 180 |
| SGLYSLSSVVTVPSSSLGTQTYICNVNHKPSNTKVDKKVEPKSCDKTHTCPPCPAPELLG | 240 |
| GPSVFLFPPKPKDTLMISRTPEVTCVVVDVSHEDPEVKFNWYVDGVEVHNAKTKPREEQY | 300 |
| NSTYRVVSVLTVLHQDWLNGKEYKCKVSNKALPAPIEKTISKAKGQPREPQVYTLPPSRD | 360 |
| ELTKNQVSLTCLVKGFYPSDIAVEWESNGQPENNYKTTPPVLDSDGSFFLYSKLTVDKSR | 420 |
| WQQGNVFSCSVMHEALHNHYTQKSLSLSPGK | 451 (SEQ ID NO: 326) |
| alemtuzumab\|Humanized\|\|L-KAPPA | |
| DIQMTQSPSS LSASVGDRVT ITCKASQNID KYLNWYQQKP GKAPKLLIYN | 50 |
| TNNLQTGVPS RFSGSGSGTD FTFTISSLQP EDIATYYCLQ HISRPRTFGQ | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | 214 (SEQ ID NO: 327) |

Finally, the FDA approved antibodies were used as the building blocks for AVEC. These included: rituximab (Rituxan); trastuzumab (Herceptin); cetuximab (Erbitux); pertuzumab (Perjeta); panitumumab (Vectibix); alemtuzumab (Campath).

### Vaccine Domains

The AVEC vaccine domains were used from two sources either as recombinant proteins with synthetic sequences expressed in E. coli, yeast, and/or human cells or as the FDA approved vaccines.

### Coding Sequences of Vaccines as Listed in Sequence Listing

The sequences were imported from the GenBank and their immunogenis sites verified with the DNAStar software to be identical as those of the FDA approved vaccines, as shown below. They were assembled as dsDNA by overlap extension. The details of specific protocols are provided in Examples and in References. Briefly, they coding sequences were cloned into the transfection vectors controlled by the promoters suitable for a particular expression system: for expression in yeast (Saccharomyces cerevisiae and/or Pichia pastoris) they were under control of the of the alcohol oxidase promoter [27] and for expression in E. Coli under control of the T7 promoter [22] and for expression in human cells (human myeloma, hepatoma, HEK293), they were under control of the cytomegalovirus promoter [22]. The expression systems and methods facilitating expression were the same as those systems described above and in Examples and in References for expression of antibodies.

TABLE
References for sequences of vaccines used in this project.

HBsAg
HBV
MENITSGFLGPLLVLQAGFFLLTRILTIPQSLDSWWTSLNFLGG
SPVCLGQNSQSPTSNHSPTSCPPICPGYRWMCLRRFITLFILLLCLIFLLVLLDYQG
MLPVCPLIPGSTTTSTGPCKTCTTPAQGNSMFPSCCCTKPTDGNCTOPIPSSWAFAK
YLWEWASVRFSWLSLLVPFVQWFVGLSPTVWLSAIWMMWYWGPSLYSIVSPFIPLL
PI
FFCLWVYI GENBANK: J02205.1 (SEQ ID NO: 328)
GGATCCCAGA GTCAGGGGTC TGTATCTTCC TGCTGGTGGC TCCAGTTCAG
GAACAGTAAA
CCCTGCTCCG AATATTGCCT CTCACATCTC GTCAATCTCC GCGAGGACTG
GGGACCCTGT
GACGAACATG GAGAACATCA CATCAGGATT CCTAGGACCC CTGCTCGTGT
TACAGGCGGG
GTTTTTCTTG TTGACAAGAA TCCTCACAAT ACCGCAGAGT CTAGACTCGT
GGTGGACTTC
TCTCAATTTT CTAGGGGGAT CTCCCGTGTG TCTTGGCCAA AATTCGCAGT
CCCCAACCTC
CAATCACTCA CCAACCTCCT GTCCTCCAAT TTGTCCTGGT TATCGCTGGA
TGTGTCTGCG
GCGTTTTATC ATATTCCTCT TCATCCTGCT GCTATGCCTC ATCTTCTTAT
TGGTTCTTCT
GGATTATCAA GGTATGTTGC CCGTTTGTCC TCTAATTCCA GGATCAACAA
CAACCAGTAC
GGGACCATGC AAAACCTGCA CGACTCCTGC TCAAGGCAAC TCTATGTTTC
CCTCATGTTG
CTGTACAAAA CCTACGGATG GAAATTGCAC CTGTATTCCC ATCCCATCGT
CCTGGGCTTT
CGCAAAATAC CTATGGGAGT GGGCCTCAGT CCGTTTCTCT TGGCTCAGTT
TACTAGTGCC
ATTTGTTCAG TGGTTCGTAG GGCTTTCCCC CACTGTTTGG CTTTCAGCTA
TATGGATGAT
GTGGTATTGG GGGCCAAGTC TGTACAGCAT CGTGAGTCCC TTTATACCGC
TGTTACCAAT
TTTCTTTTGT CTCTGGGTAT ACATTTAAAC CCTAACAAAA CAAAAAGATG
GGGTTATTCC
CTAAACTTCA TGGGCTACAT AATTGGAAGT TGGGGAACTT TGCCACAGGA TC
(SEQ ID NO: 329)
GenBank: J02205.1
AGCATACAAT CAACTATCTG GAATTCCCGA
GGACTGGGGA CCCTGTGACG AACATGGAGAACATC
GENBANK: J02560.1 (SEQ ID NO: 330)
GenBank: J02560.1
MFQDPQERPGKLPQLCTELQTTIHDIILECVYCKQQLLRREVYDFAFRDLCIVYRDGN
PYAVCDKCLKFYS KIS EYRHYCYS VYGTTLEQQYNKPLCDLLIRCINCQKPLCPEEKQ
RHLDKKQRFHNIRGRWTGRCMSCCRSSRTRRETQL GENBANK: U34135.1 (SEQ ID
NO: 331)
HPV Type 16 L1 E6
GenBank: U34135.1
HSV gB
GenBank: M14923.1
ATGTTTCAGG ACCCACAGGA GCGACCCGGA AAGTTACCAC AGTTATGCAC
AGAGCTGCAA
ACAACTATAC ATGATATAAT ATTAGAATGT GTGTACTGCA AGCAACAGTT
ACTGCGAC GT
GAGGTATATG ACTTTGCTTT TCGGGATTTA TGCATAGTAT ATAGAGATGG
GAATCCATAT
GCTGTATGTG ATAAATGTTT AAAGTTTTAT TCTAAAATTA GTGAGTATAG
ACATTATTGT
TATAGTGTGT ATGGAACAAC ATTAGAACAG CAATACAACA AACCGTTGTG
TGATTTGTTA
ATTAGGTGTA TTAACTGTCA AAAGCCACTG TGTCCTGAAG AAAAGCAAAG
ACATCTGGAC
AAAAAGCAAA GATTCCATAA TATAAGGGGT CGGTGGACCG GTCGATGTAT
GTCTTGTTGC
AGATCATCAA GAACACGTAG AGAAACCCAG CTGTAA (SEQ ID NO: 332)
VNGPLFDHSTHSFAQPPNTALYYSVENVGLLPHLKEELARFIMGAGGSGADWAVSEF
QKFYCFDGVSGITPTQRAAWRYIRELIIATTLFAS VYRCGELELRRPDCSRPTSEGLYR
YPPGVYLTYNSDCPLVAIVESGPDGCIGPRPVVVYDRDVFSILYSVLQHLAPRLAGGG
TDAPP GENBANK: M14923.1 (SEQ ID NO: 333)
MRGGGLICALVVGALVAAVASAAPAAPAAPRASGGVAATVAANG
GPASRPPPVPSPATTKARKRKTKKPPKRPEATPPPDANATVAAGHATVRAHLREIKV
E
NADAQFYVCPPPTGATVVQFEQPRRCPTRPEGQNYTEGIAVVFKENIAPYKFKATMY
Y
KDVTVSQVWFGHRYSQFMGIFEDRAPVPFEEVIDKINAKGVCRSTAKYVRNNMETT
AF
HRDDHETDMELKPAKVATRTSRGWHTTDLKYNPSRVEAFHRYGTTVNCIVEEVDA
RSV
YPYDEFVLATGDFVYMSPFYGYREGSHTEHTTYAADRFKQVDGFYARDLTTKARAT
SP
TTRNLLTTPKFTVAWDWVPKRPAVCTMTKWQEVDEMLRAEYGGSFRFSSDAISTTF
TT
NLTEYSLSRVDLGDCIGRDAREAIDRMFARKYNATHIKVGQPQYYQATGGFLIAYQP
L
LSNTLAELYVREYMREQDRKPRNATPAPLREAPSANASVERIKTTSSIEFARLQFTYN
HIQRHVNDMLGRIAVAWCELQNHELTLWNEARKLNPNAIASATVGRRVSARMLGD
VMA
VATCVPVAPDNVIVQNSMRVSSRPGTCYSRPLVSFRYEDQGPLIEGQLGENNDVRLT
R
DALEPCTVGHRGYFIFGGGYVYFEEYAYSHQLSRADVTTVSTFIDLNITMLEDHEFVP
LEVYTRHEIKDSGLLDYTEVQRRNQLHDLRFADIDTVIRADANAAMFAGLCAFFEGM
G
DLGRAVGKVVMGVVGGVVSAVSGVSSFMSNPFGALAVGLLVLAGLVAAFFAFRYV
LQL
QRNPMKALYPLTTKELKTSDPGGVGGEGEEGAEGGGFDEAKLAEAREMIRYMALVS
AM
ERTEHKARKKGTSALLSSKVTNMVLRKRNKARYSPLHNEDEAGDEDEL
AAA66440.1 (SEQ ID NO: 334)
GTCAACGGGC CCCTGTTCGA CCACTCCACC CACAGCTTCG CCCAGCCCCC
CAACACCGCG
CTGTACTACA GCGTCGAGAA CGTGGGGCTC CTGCCGCACC TCAAGGAGGA
ACTCGCCCGC
TTCATCATGG GCGCGGGGGG TTCGGGCGCT GATTGGGCCG TCAGCGAGTT
TCAAAAGTTC
TACTGTTTTG ACGGTGTTTC CGGAATCACG CCCACCCAGC GCGCCGCCTG
GCGATATATT
CGCGAGCTCA TTATCGCCAC CACACTCTTT GCGTCGGTCT ACCGGTGCGG
GGAGCTTGAG
TTGCGCCGCC CCGACTGCAG CCGCCCGACC TCCGAAGGTC TGTACCGCTA
CCCGCCGGGC
GTGTACCTCA CGTACAACTC CGACTGTCCG CTGGTGGCCA TCGTCGAGAG
CGGCCCCGAC
GGCTGCATCG GACCCCGTCC GGTCGTGGTT TACGACCGAG ACGTTTTTTC
CATCCTCTAC
TCGGTCCTGC AGCACCTCGC CCCCAGACTA GCGGGCGGCG GGACGGACGC
GCCCCCGTAG
GCCCGCCATG CGCGGGGGGG GCTTGATTTG CGCGCTGGTC GTGGGGGCGC
TGGTGGCCGC
GGTGGCGTCG GCGGCCCCGG CGGCCCCGGC GGCCCCCCGC GCCTCGGGCG
GCGTGGCCGC
GACCGTCGCG GCGAACGGGG GTCCCGCCTC CCGGCCGCCC CCCGTCCCGA
GCCCCGCGAC
CACCAAGGCC CGGAAGCGGA AAACCAAAAA GCCGCCCAAG CGGCCCGAGG
CGACCCCGCC
CCCCGACGCC AACGCGACCG TCGCCGCCGG CCACGCCACC GTGCGCGCGC
ACCTGCGGGA
AATCAAGGTC GAGAACGCCG ATGCCCAGTT TTACGTGTGC CCGCCCCCGA
CGGGCGCCAC
GGTGGTGCAG TTTGAGCAGC CGCGCCGCTG CCCGACGCGC CCGGAGGGGC
AGAACTACAC
GGAGGGCATC GCGGTGGTCT TCAAGGAGAA CATCGCCCCG TACAAATTCA
AGGCCACCAT
GTACTACAAA GACGTGACCG TGTCGCAGGT GTGGTTCGGC CACCGCTACT
CCCAGTTTAT
GGGGATATTC GAGGACCGCG CCCCCGTTCC CTTCGAGGAG GTGATCGACA
AGATTAACGC
CAAGGGGGTC TGCCGCTCCA CGGCCAAGTA CGTGCGGAAC AACATGGAGA
CCACCGCGTT
TCACCGGGAC GACCACGAGA CCGACATGGA GCTCAAGCCG GCGAAGGTCG
CCACGCGCAC
GAGCCGGGGG TGGCACACCA CCGACCTCAA GTACAACCCC TCGCGGGTGG
AGGCGTTCCA
TCGGTACGGC ACGACGGTCA ACTGCATCGT CGAGGAGGTG GACGCGCGGT
CGGTGTACCC
GTACGATGAG TTTGTGCTGG CGACGGGCGA CTTTGTGTAC ATGTCCCCGT
TTTACGGCTA
CCGGGAGGGG TCGCACACCG AGCACACCAC GTACGCCGCC GACCGCTTCA
AGCAGGTCGA
CGGCTTCTAC GCGCGCGACC TCACCACGAA GGCCCGGGCC ACGTCGCCGA
CGACCCGCAA
CCTGCTGACG ACCCCCAAGT TTACCGTGGC CTGGGACTGG GTGCCGAAGC
GACCGGCGGT
CTGCACCATG ACCAAGTGGC AGGAGGTGGA CGAGATGCTC CGCGCCGAGT
ACGGCGGCTC
CTTCCGCTTC TCCTCCGACG CCATCTCGAC CACCTTCACC ACCAACCTGA
CCGAGTACTC
GCTCTCGCGC GTCGACCTGG GCGACTGCAT CGGCCGGGAT GCCCGCGAGG
CCATCGACCG
CATGTTTGCG CGCAAGTACA ACGCCACGCA CATCAAGGTG GGCCAGCCGC
AGTACTACCA
GGCCACGGGG GGCTTCCTCA TCGCGTACCA GCCCCTCCTC AGCAACACGC
TCGCCGAGCT
GTACGTGCGG GAGTACATGC GGGAGCAGGA CCGCAAGCCC CGGAATGCCA
CGCCCGCGCC
ACTGCGGGAG GCGCCCAGCG CCAACGCGTC CGTGGAGCGC ATCAAGACCA
CCTCCTCGAT
CGAGTTCGCC CGGCTGCAGT TTACGTATAA CCACATACAG CGCCACGTGA
ACGACATGCT
GGGGCGCATC GCCGTCGCGT GGTGCGAGCT GCAGAACCAC GAGCTGACTC
TCTGGAACGA
GGCCCGCAAG CTCAACCCCA ACGCCATCGC CTCCGCCACC GTCGGCCGGC
GGGTGAGCGC
GCGCATGCTC GGAGACGTCA TGGCCGTCGC CACGTGCGTG CCCGTCGCCC
CGGACAACGT
GATCGTGCAG AACTCGATGC GCGTCAGCTC GCGGCCGGGG ACGTGCTACA
GCCGCCCCCT
GGTCAGCTTT CGGTACGAAG ACCAGGGCCC GCTGATCGAG GGGCAGCTGG
GCGAGAACAA
CGACGTGCGC CTCACCCGCG ACGCGCTCGA GCCGTGCACC GTGGGCCACC
GCGGCTACTT
CATCTTCGGC GGGGGCTACG TGTACTTCGA GGAGTACGCG TACTCTCACC
AGCTGAGTCG
CGCCGACGTC ACCACCGTCA GCACCTTCAT CGACCTGAAC ATCACCATGC
TGGAGGACCA
CGAGTTTGTG CCCCTGGAGG TCTACACGCG CCACGAGATC AAGGACAGCG
GCCTGCTGGA
CTACACGGAG GTCCAGCGCC GCAACCAGCT GCACGACCTG CGCTTTGCCG
ACATCGACAC
GGTCATCCGC GCCGACGCCA ACGCCGCCAT GTTCGCGGGG CTGTGCGCGT
TCTTCGAGGG
GATGGGGGAC TTGGGGCGCG CGGTCGGCAA GGTAGTCATG GGAGTAGTGG
GGGGCGTGGT
GTCGGCCGTC TCGGGCGTGT CCTCCTTTAT GTCCAACCCC TTCGGGGCGC
TTGCCGTGGG
GCTGCTGGTC CTGGCCGGCC TGGTCGCGGC CTTCTTCGCC TTCCGCTACG
TCCTGCAACT
GCAACGCAAT CCCATGAAGG CCCTGTATCC GCTCACCACC AAGGAACTCA
AGACTTCCGA
CCCCGGGGGC GTGGGCGGGG AGGGGGAGGA AGGCGCGGAG GGGGGCGGGT
TTGACGAGGC
CAAGTTGGCC GAGGCCCGAG AAATGATCCG ATATATGGCT TTGGTGTCGG
CCATGGAGCG
CACGGAACAC AAGGCCAGAA AGAAGGGCAC GAGCGCCCTG CTCAGCTCCA
AGGTCACCAA
CATGGTTCTG CGCAAGCGCA ACAAAGCCAG GTACTCTCCG CTCCACAACG
AGGACGAGGC
CGGAGACGAA GACGAGCTCT AAGGGAGGGG AGGGGAGCTG GGCTTGTGTA
TAAATAAAAA
GACACCGATG TTCAAAAATA CACA (SEQ ID NO: 335)

### FDA Approved Vaccines.

The vaccines approved by the FDA were used to engineer AVEC as AVEC vaccine domains and to use as the references: Engerix, Recombivax, Gardasil, Cervarix.

### Linkers

The AVEC functional domains are integrated with the aid of three methods: bifunctional chemical linkers aka spacers, and amino acid (AA) linkers and spacers by recombinant expression from DNA sequences coding for AA linkers.

### Bifunctional Chemical Linkers.

Linkers facilitating integration of the aforementioned antibodies and vaccines are listed below. They are used as homo- or hetero-specific linkers depending if the functional groups aimed at docking the linkers are the same in the antibody and the vaccine or different, i.e., choosing from the combinations of NH2-, SH—, COOH—. The specific protocols of using them are provided by their manufacturers, while some of them as are outlined including, but not limited, as shown below in Examples and References. [28, 31, 32].
A Linker SMBC
B Linker SMCC
C Linker EDC
D Linker PTAD
E Linker BMC
F Linker NHS ester
G Linker isothiocyanate
H Linker benzoyl fluorides I Linker maleimide
J Linker iodoacetamide K Linker thiopyridines
L Linker arylopropiolonitrile
M Linker diazonia

### Amino Acid (AA) Linkers

The AA linkers are incorporated into AVEC by two main biomolecular strategies: either after synthesis of transgenic expression of the AA oligopeptide they are modified with the bifunctional linkers described above, or the DNA coding sequences are designed to be inserted into the DNA constructs between the coding sequences for the antibody and the vaccine, if the AVEC are expressed as the fusion proteins. The AA linkers' functions', in addition to integrating, involve creating a flexible spacer, changing hydrophobicity/hydrophilicity of AVEC, altering half-life of AVEC, promoting or reducing immunogenicity of AVEC. In some DNA constructs coding for AVEC as fusion proteins, the DNA sequences were omitted altogether; thus resulting in AVEC as the fusion protein comprising directly connected domains: the antibody and the vaccine. Some of the linkers tested are listed below, while the specific details of the methods are provided in References and Examples. The AA sequences are provided in the one letter code with p,q,r,s,t,u being the numbers: 1-1000.
A Linker (SmSnSoGp)q
B Linker PEG 0-100 kDa
C Linker 6-aminohexanoic acid: H2N—(CH2)p-COOH
D Linker H2n-(CH2)q-NH2
E Linker COOH—(CH2)r-NH2
F Linker COOH—(CH2)s-COOH
G Linker COOH—(CH2)t-SH
H Linker SH—(CH2)u-NH2

### DNA Sequences Coding for AA Linkers

DNA sequences were synthesized as oligonucleotides. They were either expressed in the human expression systems outlined above as oligopeptides and used as linkers as described above or incorporated in the antibody and vaccine expression vectors described above according to the standard detailed protocols in References and Examples.

### Reporters

The molecules having specific features facilitating their detection with the dedicated instruments were incorporated into AVEC as to report their presence, location, concentration, absorption, distribution, metabolism, clearance in/by cells and in the patients' bodies. They are incorporated directly into AVEC or through binding domains (e.g., metal binding domains, or chelating domains) relying upon the standard detailed protocols as outlined in the manufacturers' recommendations, published in the peer-reviewed journals, cited in References, and described in Examples. In some embodiments, they comprise the fluorochromes, magnets, noble metals, radionuclides, and bubbles.

### Reporters Comprising Magnets for Magnetic Activation Cell Sorting (MACS), Nuclear Magnetic Resonance (NMR), Magnetic Resonance Imaging (MRI)

A Reporter domain comprising Gd,
B Reporter domain comprising Eu,
C Reporter domain comprising Fe,
D Reporter domain comprising Ni,
E Reporter domain comprising Co.
   Reporters Comprising Fluorochromes for Detection with Flow Cytometry (FCM), Fluorescence Activated Cell Sorting (FACS), Whole Body Fluorescent Imaging
A Reporter domain comprising FITC, B Reporter domain comprising RITC,
C Reporter domain comprising rhodamine(s), D Reporter domain comprising TR,
E Reporter domain comprising indocyanin green,
F Reporter domain comprising phycoerythrins,
G Reporter domain comprising Alexa(s), H Reporter domain comprising BODIPYs, I Reporter domain comprising DRAQs,
J Reporter domain comprising CYTRAK, K Reporter domain comprising coumarins, L Reporter domain comprising Pacific(s), M Reporter domain comprising Cy(s),
N Reporter domain comprising IRD(s), O Reporter domain comprising Tb
P Reporter domain comprising Eu
P Reporter domain comprising Green fluorescent proteins and their modifications.

### Reporters Comprising Noble Metals for Energy Dispersion X-Ray Spectroscopy (EDX), Total Reflection X-Ray Fluoroscopy (TXRF), X-Ray Imaging, Raman, Mass Spectroscopy, Computed Tomography (CT)

A Reporter domain comprising Au,
B Reporter domain comprising Pt,
C Reporter domain comprising Pd,
D Reporter domain comprising Ag.

### Reporters Comprising Radionuclides for Gamma-Scintigraphy, Single Photon Emission Tomography (SPECT), Positron Emission Tomography (PET)

A Reporter domain comprising Tc99m,
B Reporter domain comprising I125,
C Reporter domain comprising I131,
D Reporter domain comprising F18,
E Reporter domain comprising Cu64.

### Utility of AVEC for Therapy

The two types of studies are conducted to demonstrate safety and efficacy of AVEC in therapy of cancer patients: in vitro ex vivo and in vivo.

In vitro ex vivo studies are conducted by establishing tissue cultures as models of in vivo tissues being encountered by AVEC. As reported above, in the peer-reviewed articles, Examples, and References, these human artery and vein endothelial cells, human normal breast tissues, human normal ovary surface cells, human normal prostate tissue, white blood cells, erythrocytes, platelets, etc. For the biopsies of these cells from the specific patients, the results of these studies can be included into the diagnostic process, while designing the dose and regimens of therapy, as the precision, personalized medicine.

In vivo studies involve procedures considered to be minimally invasive surgery: subcutaneous injections and intravenous infusions. They are always preceded by presenting the Patient Bill of Rights, by receiving the Patient Informed Consent, and being conducted by the physician.

### EXAMPLES

Having described the invention with reference to the embodiments and illustrative examples, those in the art may appreciate modifications to the invention as described and illustrated that do not depart from the spirit and scope of the invention, which is fully original, not occurring in nature, and not obvious, as disclosed in the specification. The examples are set forth to aid in understanding the invention, but are not intended to, and should not be construed to limit its scope in any way. The examples do not include detailed descriptions of conventional methods, instruments, sequences, and reagents, which are referenced to the published literature describing previous art. Such methods are well known to those of ordinary skill in the art and are described in numerous publications referenced herein. Further, all references cited above and in the examples below are hereby incorporated by reference in their entirety, as if fully set forth herein.

### Example 1: Patients

Blood and cancer biopsies were acquired from the ten patients suffering from the advanced breast, ovarian, colorectal, prostate, and lung cancers, from the Acute and Chronic Infection with Hepatitis B virus, Herpes Simplex virus, Human Papilloma virus, Epstein Barr virus, Cytomegalovirus, and from the healthy volunteers having various titers of antibodies induced by standard HBV or HPV vaccinations. All biopsies were acquired in accordance with the Declaration of Helsinki, Institutional Review Board approval, and with Patients' Informed Consent (PIC), who were first presented with the Patient Bill of Rights. In each and every example described herein, the aforementioned procedure was thoroughly pursued. All the procedures were in strict compliance to the standards of Good Medical Practice and conducted by physicians serving under the Hippocratic Oath.

### Example 2: Generation of Antibodies as Antibody Domains of AVEC

For generating of anti-HBsAg, anti-HBcAg, anti-HBcAg antibodies the B cell were acquired from the patients suffering from the Acute and Chronic Hepatitis B. The protocol was identical to that published [21, 22, 31, 32]. Dane particles and HBV, isolated from the patients' blood by PEG gradients precipitation or from liver biopsies by CsCl gradient centrifugations, were rapidly frozen, lyophilized and stored.

Biotechnology of human anti-HER-2 antibodies synthesis was pursued by adaptation of that originally described, either as new antibodies or as biosimilars to the FDA approved trastuzumab, pertuzumab, cetuximab, panitumumab, alemtuzumab, rituximab. The FDA approved: trastuzumab, pertuzumab, cetuximab, panitumumab, alemtuzumab, rituximab were included into the project as the references. For designing of the DNA plasmid constructs for the aforementioned biosimilar antibodies the coding sequences were imported from the ImMunoGeneTics antibody sequences' bank (IMGT, Paris, F, EU).

HBsAg, gB, gH, gD, gL, HER-2, EGFR, EpCAM, p350, VP16, VP18 were produced in human hepatoma or myeloma cells transfected with plasmid DNA. Prior to selection, during in vitro evolution, they were reconstituted with buffer and served as the molecular baits. They also served as the negative controls for antibodies.

For generating of anti-HSVgB and anti-HSVgD antibodies the B cell were acquired from the patients suffering from the Herpes Simplex. The protocol was identical to that described above.

For generating of anti-HPV-VP 16 and 18 antibodies the B cell were acquired from the patients suffering from the Human Papilloma Virus. The protocol was identical to that described above.

Designing, engineering, and manufacturing such AVEC marker binding domains may be accomplished as follows. IgG, scFv, dsFvs, sdFvs, and CDRs against were constructed by generating combinatorial display libraries using HEK293 cell and mRNA displays according to published protocols (31, 32).

The cancer patients' blood was drawn as small aliquots under the informed consent based upon the IRB approved protocol. To 2 ml of anticoagulant-treated blood, 2 ml of balanced salt solution were added and mixed. Unto the top of 3 ml of the Ficoll-Paque Plus in Falcon tube, 4 ml of diluted blood were layered without mixing. The samples were centrifuged at 400 g for 30-40 minutes at 18-20° C. This led to separation of the sample into four layers: 1. plasma (top), 2. lymphocytes, 3. Ficoll-Paque Plus, and 4. granulocytes, erythrocytes. After discarding the plasma, the lymphocyte layer was transferred to the new Falcon tube, to which at least 3 volumes of balanced salt solution were added and mixed. The sample was centrifuged at 400 g for 10 minutes at 18-20° C. The supernatant was removed. The lymphocytes were resuspended in 6-8 ml balanced salt solution. The cells were counted on the Beckman Coulter cell counter with forward scattering indicative of cells' sizes and side scattering indicating their viability granularity.

The B cells were isolated by negative selection. Non-B cells, i.e., T cells, NK cells, monocytes, dendritic cells, granulocytes, platelets, and erythroid cells depletion was performed with antibodies against CD2, CD14, CD16, CD36, CD43, and CD23 tagged with our magnetic beads. This left the sample with a pure population of untouched B cells. This was validated by labeling of B cells with CD19 and CD20. The samples were further processed or stored in liquid nitrogen.

After extracting total RNA from the isolated lymphocytes Trizol (MRC) according to published protocols [22], RT-PCR was performed to amplify human antibody complementarity determining regions (CDRs) and framework regions (FRs). cDNA was prepared using SuperScript™ III First-Strand Synthesis System (Invitrogen). Alternatively, cDNA was obtained by Cells-To-cDNA kit from Qiagen. Approximately, 5 pg to 25 pg of RNA or mRNA was reverse transcribed into the first-strand cDNA.

The CDR and FR cDNA was then amplified by PCR. The primers were selected from those published as listed above.

Using the cDNA and combinations of these primers, the CDRs and FRs from cDNA samples were amplified using standard PCR protocols including preparing the following mixture in PCR multi well plates: ddH2O, 2× High Fidelity PCR Master, Forward primer, Reverse primer, and cDNA; and cycling on ABI 7900 or 7500 FAST: (a) 4 min at 94° C.; (b) 45 sec at 94° C.; 45 sec at 55° C.; 1 min at 72° C.×30 cycles; (c) 5 min at 72° C.

The amplicons were run on 2% agarose gel, stained with SybrGold, and imaged with Storm 840. These primers were either cloned under used for diversification after PCR introducing the following restriction sites:
Sfi I: 5′ GGCC NNNN*N GGCC . . . 3′; and
Sacll: 5′ CCGC*GG . . . 3′;
   and then assembled into single chain variable fragments (scFv), single domain variable fragments (sdFv), dual chain variable fragments (dcFvs), or complementary domain regions (CDRs) within the human IgG framework.

HEK293, Phage, and mRNA Displays.

The PCR amplicons digested with the sril and Sacll (New England Biolabs, Ipswich, Mass.), gel purified, and ligated into the pDisplay (Invitrogen), which contains a PDGFR anchor. The ligation mix was used to transform E. coli TOP10 cells (Invitrogen). Each transformation produced surface display library containing −10A6 clones. This was further diversified. DNA was recovered with Miniprep from Qiagen.

HEK293T cells were grown in DMEM with DCS and were transfected using Lipofectamine Plus. After 72 h, they were labeled with antimyc and purified receptor protein ged with magnetic beads or fluorochromes. This allowed isolation of positive expressors from the medium. DNA was recovered from each clone in preparation for determination of affinity constant after HEK293T expression and for sequencing.

Phagemid pComb3X cut with Sfil was used to clone CDR and FR after multiple rounds of PCR with overlap extension and to get CDRs and FRs together. The inserts were ligated into the vector with T4 ligase followed by desalting with Amicon Ultra-4. TG1 electroporation-competent cells were transfected with desalted ligations by electroporation and grown in 2YT medium. Qiagen HiSpeed Plasmid Maxi Kit was used for phagemid preparation. mRNA display and expression was performed as previously described. Selection of internalizing vs non-internalizing clones of scFv, dcFv, sdFv, IgG, Fab, (Fab)2 was performed as previously described.

### Example 3: Biomolecular Engineering of HBsAg

HBsAg was isolated from the patients suffering from Acute and/or Chronic Hepatitis B: either from the blood by PEG fractionations or from the liver biopsies by CsCl gradient centrifugation. To assure exact immunogenic compatibility with the immunity induced by vaccinations with the FDA approved HBsAg, which were produced in yeast, the HBsAg in this project were also generated in yeast as originally described. Biotechnology of the recombinant HBsAg was pursued based upon the published DNA coding sequence. Hepatitis B virus like particles (VLP) were initially synthesized in yeast—Saccharomyces cerevisiae as originally described. In particular, the expression plasmid pHBS-16 included the HBsAg surface antigen (HBsAg) controlled by the yeast alcohol dehydrogenase (ADHI) promoter through introduced by EcoRI restriction sites into the DNA construct of the pBR322 plasmid. That followed by yeast replication origin, yeast trp1 gene. This biotechnology was later modified to be pursued in Pichia pastoris. Briefly, yeast cultures of Pichia pastoris were grown at 30° C. in rich medium (YPD; 1% yeast extract, 2% bactopeptone, 2% glucose) initially and shifted either to synthetic media (YNM, 0.67% yeast nitrogen base supplemented with 0.5% (v/v) methanol) for immunoprecipitation and immunofluorescence experiments, or to mineral media (MMOT, 0.2% (v/v) oleate and 0.02% (v/v) Tween-40) for fractionation studies. All the protocols' products—HBsAg VLPs were referenced and validated to the FDA approved and the CDC recommended Engerix B and Recombivax and standard clinical diagnostics.

### Example 4: Biotechnology of Mimotopes

Design of HER-2 cyclic mimotopes was initiated by importing the DNA from the GenBank and in vitro translation into amino acid sequences or direct amino acid sequences from SwissProt into the Peptide 3D or Laser-Gene software. That followed by determination of surface displayed domains. Further analysis led to selection of the most likely immunogenic domains. The 12-40 amino acids long sequences were selected. The amino acid sequences were exported directly into the program of the peptide synthesizer (ABI, Foster City, Calif.). The selected sequences were altered by introducing glycine linkers with terminal cysteines at both amino and carboxyl terminus of the peptide designs. The designed peptides were synthesized as linear on the peptide synthesizer. After detachment from the cartridges, the peptides were converted into cyclics by means of the cysteines. The synthetic products—HER-2 mimotopes were selected on the high pressure liquid chromatography columns. The specificity of the mimotopes was validated by binding to trastuzumab and ant-HER-2 antibodies with the aid of MACS or FACS.

### Example 5: Biomolecular Engineering of AVEC: Anti-Her-2×HBsAg

The synthetic anti-HER-2 antibodies and synthetic HBsAg VLPs were linked with heterospecific, bifunctional linker: sulfo-m-maleimidobenzoyl-N-hydroxysuccinimide hydroxysuccinimide ester (SMBS). Briefly, the anti-HER-2 antibody was dialyzed against 0.15 M sodium chloride, 0.1 M sodium phosphate, at pH 7.2.

Sulfo-MBS stock in DMSO was added to this solution up to the final 2% w/v concentration to assure at least 80× molar excess. After 1 h at room temperature, the reaction solution was rapidly applied to desalting columns. Performing chromatography with the 0.15 M sodium chloride, 0.1 M sodium phosphate, at pH 7.2 carrier solution was followed by pooling the activated anti-HER-2 antibodies 1 ml fractions. To this solution, the synthetic HBsAg diluted in the same carrier solution was promptly added to assure 1:1 ratio. The reaction continued for 1 h at room temperature. The effective anti-HER-2×HBsAg clusters were isolated by chromatography. The specificity of the anti-HER-2×HBsAg to label HER-2 receptors was validated by FCM on cells and by NMR and XRFS on mimotopes. The specificity of the anti-HER-2×HBsAg to attract immune response was validated by labeling with anti-HBsAg antibodies rendered fluorescent for FCM or superparamagnetic for NMR.

### Example 6: Cell Cultures

Many cell lines have been used described herein. Examples of such cell lines shown are shown in Table 1, and were grown in media recommended by ATCC in incubators (New Brunswick, Fisher, Napco) in saturated humidity, 37 deg C., 5% CO2. The cell lines were selected to cover the entire spectrum of lineages (including cancers of breast, ovary, colorectum, prostate, lymphoid, lung, testis, pancreas, cervix, liver, and brain), malignancy, metastasis, expression of receptors, and presence of mutations. On all these cell lines, the proof of concept was validated as described herein. All cell lines were obtained from ATCC unless otherwise noted.

TABLE 1
AVEC TESTED CELL LINES INDEXED AT ATCC

| | |
|---|---|
| Cell Lines that | SKBR3 from ATCC as HTB30 (overexpressed strongly) |
| Overexpress | UACC893 (20x gene amp) |
| HER2 | UACC812 (15x gene amp) |
| | CRL2338 from ATCC with designation HCC1954 (overexpressed strongly) |
| | AU565 from ATCC as CRL2351 (overexpressed strongly) |
| | MAC117 (gene amp 7x) |
| | MDA-MB453 (a bit more than MCF7, just above base ~3x) |
| | BT474 from ATCC as CRL |
| | CRL2340 from ATCC HCC2157 |
| | HCC2218 from ATCC as CRL2343 |
| | BT483 |
| Cell Lines that | HTB22 from ATCC with designation MCF7 (base) |
| express a Basal | HBL100 |
| Levels of HER2 | MB231 |
| | HCC202 (basal or overexpressed) |
| | CRL 2320 HCC1008 from ATCC as (basal or overexpressed) metastatic |
| | NCI-H23 (basal or overexpressed) lung cancer |
| Cell Lines that | CRL2314 from ATCC with designation HCC38 |
| are negative for | CRL2315 HCC70 from ATCC |
| HER2 | CRL2321 HCC1143 from ATCC |
| | CRL2322 HCC1187 from ATCC |
| | CRL2324 HCC1395 from ATCC |
| | CRL2326 HCC1419 from ATCC |
| | CRL2327 HCC1428 from ATCC |
| | CRL2329 HCC1500 from ATCC |
| | CRL2330 HCC1569 from ATCC |
| | CRL2331 HCC1599 from ATCC |
| | CRL2336 HCC1937 from ATCC as (BRCA mut) |
| | CRL2343 HCC2218 from ATCC |
| Cell Lines that | HBE135-E6E7 from ATCC as CRL2741 (also high TGF) bronchial ducts |
| Overexpress | A431 |
| EGFR | |
| Cell Lines that | CRL2918 from ATCC designation Nm2C5 EGFR pos (basal or over) |
| express a Basal | CRL2919 from ATCC designation Nm2C5 gfp EGFR pos (basal or over) |
| Levels of EGFR | M4A4 (basal or over) |
| | NCI-H23 (basal or over) |
| | Mutation A750del in EGFR CRL2868 adenocarcinoma |
| | Mutation A751del in EGFR CRL2869 adenocarcinoma |
| | Mutation A751del in EGFR CRL2871 adenocarcinoma |
| | HTB127 from ATCC with designation MDA-MB-330 (basal or over) |
| | HTB132 from ATCC with designation MDA-MB-468 (basal or over) |
| | HTB26 from ATCC designated MDA_MB_231 (basal or over) |
| Reference Cell | EGFR A431 2-6 × 10≢receptors per cell |
| Lines | HER2 BT474 6-10 × 10≡receptor per cell |
| | EGFR Normal breast primary culture 8% of A431 |
| | HER2 Normal breast primary culture 3% of A431 |
| Cell Line with a | UG87mutant |
| Mutation of | |
| EGFRvIII | |

The cell line CRL-2340 HCC2157 was derived from the ductal carcinoma of the mammary gland tumor classified as TNM se lilA, grade 2, with lymph node metastasis. The cells were grown in a 1:1 mixture of Ham's F12 medium with 2.5 mM L-glutamine and Dulbecco's Modified Eagle's Medium adjusted to contain 1.2 gIL sodium bicarbonate with additional supplements (ATCC).

The cell line MCF7 HTB-22. The cells are positive for estrogen receptor and express WNT7B oncogene. The medium to culture this cell line is Eagle's Minimum Essential Medium (ATCC) with these added components: 0.01 mg/ml bovine insulin; donor bovine serum to a final concentration of 10%.

The cell line 184A1 CRL-8798 was originally established from normal mammary tissue and was transformed to benzopyrene. The line appears to be immortal, but is not malignant. The line grows in Mammary Epithelial Growth Medium (MEGM) (Clonetics) supplemented with 0.005 mg/ml transferrin and 1 ng/ml cholera toxin.

Several of the cell lines used in the experiments described herein are further described in more details. The cell lines TOV-112D CRL-11731 and CRL-11732 OV-90 were derived from primary malignant adenocarcinomas of the ovary at grade 3, se IIIC. They were cultured in a 1:1 mixture of MCDB 105 medium and Medium 199, 85%; donor bovine serum 15% (ATCC). The cells were tumorigenic in nude mice. They formed colonies and spheroids when cultured in soft agar. The cells tested positive for HER2/neu and p53 mutation.

The cell line NIH OVCAR-3 HTB-161 was derived from the cells in ascites of a patient with malignant adenocarcinoma of the ovary. The cell line was grown in RPMI-1640 Medium (ATCC) supplemented with 0.01 mg/ml bovine insulin and donor bovine serum to a final concentration of 20%. The epithelial cells were positive for estrogen and progesterone receptor. They formed tumors in nude mice.

The normal, adherent fibroblast cell line Detroit 573 CCL-117 was derived from skin. It is grown in Minimum essential medium (Eagle) in Earle's BSS with nonessential amino acids (ATCC), sodium pyruvate (1 mM) and lactalbumin hydrolysate (0.1%), 90%; fetal bovine serum, 10%. The cells were grown into spheroids within a synthetic extracellular matrix.

Viability Tests and Doubling Times.

The cells were stained with Hoechst vs PI and counted on Beckman Coulter flow cytometer to determine ratios between total number of cells and dead cells at 24 hour intervals to determine doubling times and viability.

Selection of Clones with High Metastatic Potential.

For the in vitro studies described herein, cell lines described above were grown as described above. They were resuspended and spilled over the endothelial cells grown over extracellular basement membrane as described in the details previously (22). After short incubation at 37 deg. C., the cells cultures were rinsed with media, while removing non-adherent cancer cells. The attached cells were resuspended again and split into single clones grown in multiwell plates. These enriched clones were used for further studies because they imitated the metastatic clones of the lines derived from the primary tumor.

Immunolabeling.

Cell spheroids grown in the culture were spun down at 300×g. The cells were resuspended in the donor serum or whole blood to which superparamagentic scFv were added. Upon completion of labeling, the cells were rinsed with PBS. They were studied with CT, MRI, USG, FL, RSI, PET, SPECT, or NMR or alternatively processed by freezing in preparation for laser scanning confocal microscopy (LSCM) or EDXSI or EELS. Alternatively, cell lysates electrotransferred onto PVDF membranes were immunolabeled with scFv with or without chelated metal atoms.

Freezing and Freeze-Substitution of Cell Spheroids.

The details of cryoimmobilization of cultures of cell spheroids by freezing are described previously and are only briefly presented here (21). Briefly, cells were injected into chambers were rapidly frozen in nitrogen slurry down to down to −196° C. The frozen samples were placed into methanol that was precooled to −90° C. in the freezer (ThermoNoran).

Temperatures were maintained at −90° C., −35° C., and 0° C. for 48 hours. Infiltration with Lowicryl preceded polymerization with UV at −35° C. and ultramicrotomy. Alternatively, critical point drying was followed by fast atom beam sputter coating (lonTech).

Native Electrophoresis.

A 2% agarose gel was poured using a 10 mM Tris, 31 mM NaCl buffer of varying pH that did not contain any denaturing agents. The samples in their native state were loaded after being mixed with glycerol to add density without denaturing the proteins. The gel was run in the same buffer used for pouring the agarose at 60 mAmps until the desired separation was reached as determined by the presence of fluorescent markers with a molecular weight higher and lower than the scFv tested. The gel was then stained for 30 minutes in Sypro Tangerine Gel Stain (Invitrogen) diluted in the running buffer before imaging using a FluorImager (Molecular Dynamics).

SDS-PAGE.

Electrophoresis was run on 12% polyacrylamide gel. Several 0.75 thick combs with the 2 mm lanes were loaded with standard, cell culture Iysates. The samples, after mixing with SDS and DTT containing sample buffers (Sigma) were loaded into the wells. The gels were run using a Tris/Glycine/SDS/DTT running buffers. After the run, the gels were stained with colloidal silver or Sypro Tangerine for imaging using Storm 840 or FluorImager (Molecular Dynamics).

Electrotransfer.

After electrophoresis, the samples were immediately transferred onto PVDF. The immunoblotting was performed with the Mini Trans-Blot Cell (Bio-Rad) within CAPS: 10 mM 3-[Cyclohexylamino]-1-propanesulfonic acid (CAPS), Tris/glycine transfer buffer 25 mM Tris base, 192 mM glycine, pH 8.3. Prior to the transfer, the cooling units were stored with deionized water at −20 C. Immediately after electrophoresis the gel, membrane, filter papers and fiber pads were soaked in transfer buffer for 5-10 minutes. The pre-cooled transfer units were filled with cooled transfer buffer. Alternatively, magnetic field generator was approached by the tube or plate containing an aliquot of the patient's blood supplemented with AVEC. The labeled cells were retained, while the blood withdrawn. After rinsing with PBS, the labeled cancer cells were retained for further studies on the counting chamber, fluorometer, and/or confocal.

Laser Scanning Confocal Microscopy.

(LSCM) The three-dimensional stacks of the cells labeled with AVEC were imaged with the Olympus or Leica laser scanning confocal systems. Excitation wavelengths were used: 337, 488, 543, and 588 nm. Alternatively, reflected or Raman optics were used. Images were acquired with Kernel filtration and deconvolution of the data was followed by 3D or cascade display for analysis.

Spectral Mapping Using Energy Dispersive X-Ray Analysis Spectroscopic Imaging (EDXDI) and Electron Energy Loss Spectroscopic Imaging (EELSI).

Supramolecular architecture analysis of the AVEC was performed with Field Emission Scanning Electron Microscope with Energy Dispersive X-Ray Spectral Imaging System (EDXSI)—Hitachi 3400. Complete elemental spectra were acquired for every pixel of the scans to create the elemental databases. From them, after selecting an element specific energy window, the map of this element atoms distribution was extracted and ZAF correction calculated (NIST). As AVEC were ged with superparamagnetic metal particles (nanoclusters or core-shell nanoparticles) or noble metal nanoparticles were ged or incorporated into their structures, their location was determined based upon spectral elemental maps superimposed over molecular architecture with zero loss or carbon edge tuning (21). Purity of elemental composition and geometry of gold nanoparticles were evaluated with EOXSI using Vacuum Generators 501, Hitachi 5900, and JEOL 1540 instruments under control of Gatan, Voyager software.

X-Ray, Atomic Absorption Spectroscopic, Surface Plasmon Resonance Detection, Centrifugation, and Selection.

One molecule of AVEC with one gold nanoparticle having about 100-1000 atoms of gold with the diameter 1.59 A and mass 197 amu each increased mass of scFv ged up to 19,9660a and that is made of about 1000 atoms up to 196,6670a. Separation of AVEC markers from blood via centrifugation in response to gravity during centrifugation at low g, compared to unlabeled ones. This did lead to very simple and rapid separation of AVEC markers from the aliquot of the patient's blood.

CT—Computed X-Ray Tomography.

For evaluating relative contrast agents in CT, solutions of 1M, 0.1 M, 0.01 M, and 0.001 M, 0.0001 M sodium iodide, calcium chloride, gold chloride, and gold nanoparticles of various sizes in deionized water were dispensed into the wells of microarray plates. Additional rows contained blood, physiological saline, while an additional row was left empty, i.e., to contain air. Computed tomography was pursued with Toshiba Aquilion 64-slice clinical scanner. Initial settings were as follows: vole 120 peak kV, current 40 mA, exposure time of 0.6 s, slice setting 0.5 mm (the slices that were thereafter compressed into 2 mm display images), (modifications of these settings were indicated in the figure legends). ImageQuantTL® version 1.1.0.1 was used to evaluate relative peak pixel intensity of the samples on the computed tomography images utilizing a 0 to 255 level grayscale. The Aquilion scanner may also record phantoms for use in detecting biomarker density by measuring the signal intensity of the AVEC in Haunsfield units.

Nuclear Magnetic Resonance and Selection.

The wide-bore nuclear magnetic resonance (NMR) spectrometer operated at 9T (Brucker) with a mouse-cage resonator was used to evaluate relative relaxivity of the samples based upon T1 measurements. T1 spin lattice relaxation time calculated using inversion recovery pulse sequence was measured using inversion recovery imaging with Tl=50-4000 ms in 100 ms increments. T1 was also calculated from T1-weighted fluid-attenuated inversion recovery (T1-FLAIR) sequence (TrITe/Flip=2210/9.6/90), as well as standard T1 weighted imaging sequences (TrITe/Flip=400/6/90). For studies of labeling in vitro, a small table top NMR spectrometer was used at 0.5T. After labeling with superparamagnetic scFv, the blood sample containing labeled cancer cells was injected into microfluidic channel of 20 micron in diameter, which was placed with the field. Passage of the single cell, which was labeled with superparamagnetic scFv, was determined by the spectral response and recorded.

### Reporter Domains

Reporter domains that may be used in accordance with the embodiments described herein may include, but are not limited to ions and nanoparticles, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions), or a fluorochrome or a microbubble or a radionuclide.

In one embodiment, the reporter component is a metal nanoparticle. The metal nanoparticles may be formed from a single suitable solid metal or from a combination of two or more suitable metals.

In some embodiments, the metal nanoparticle may comprise a nanoparticle derived from a noble metal, including, but not limited to, Gold (Au), Platinum (Pt), Palladium (Pd) and Silver (Ag).

In other embodiments, the metal nanoparticle may comprise a superparamagnetic metal, including, but not limited to, Europium (Eu), Gadolinium (Gd), Iron (Fe), Nickel (Ni) or Cobalt (Co).

In other embodiments, the metal nanoparticle may comprise a nanoparticle derived from a fluorescent metal, including, but not limited to, Europium (Eu) and Terbium (Tb).

Some metal nanoparticles can be made as chelated nanoclusters or as coreshell nanoparticles, which have a superparamagnetic, heavy metal or fluorescent metal core that is sealed inside a noble metal layer These metals include ions of Cr, Va, Mn, Fe, Co, Ni, Cu, La, Ce, Pr, Nd, Pm, Sm, Eu, Gd, Tb, Dy, Ho, Er, Tm, Yb, Ru, and Lu.

Other nanoparticles may be made as a “microbubble” nanoparticle, having a noble metal outer layer, with a hollow core.

In addition, it should be noted that some nanoparticles, for example, quantum dots, may also be suitable for use as a detection agent.

Radioactive substances that may be used as a reporter component in accordance with the embodiments of the disclosure include, but are not limited to, .sup.18F, .sup.32P, .sup.33P, .sup.45Ti, .sup.47Sc, .sup.52Fe, 59Fe, .sup.62Cu, .sup.64Cu, .sup.67Cu, .sup.67Ga, .sup.68Ga, .sup.75Sc, .sup.77As, .sup.86Y, .sup.90Y. .sup.89Sr, .sup.89Zr, .sup.94Tc, .sup.94Tc, .sup.99mTc, .sup.99mMo, .sup.105Pd, .sup.105Rh, .sup.111Ag, .sup.111In, .sup.123I, .sup.124I, .sup.125I, .sup.131I, .sup.142Pr, .sup.143Pr, .sup.149Pm, .sup.153Sm, .sup.154158Gd, .sup.161Tb, .sup.166Dy, .sup.166Ho, .sup.169Er, .sup.175Lu, .sup.177Lu, .sup.186Re, .sup.188Re, .sup.189Re, .sup.194Ir, .sup.198Au, .sup.199Au, .sup.211At, .sup.211Pb, .sup.212Bi, .sup.212Pb, .sup.213Bi, .sup.223Ra and .sup.225Ac.

Paramagnetic ions that may be used as reporter components in accordance with the embodiments of the disclosure include, but are not limited to, ions of transition and lanthanide metals (e.g. metals having atomic numbers of 6 to 9, 21, 29, 42, 43, 44, 57, 71). These metals include ions of Cr, V, Mn, Fe, Co, Ni, Cu, La, Ce, Pr, Nd, Pm, Sm, Eu, Gd, Tb, Dy, Ho, Er, Tm, Yb, Ru, and Lu.

### Example 7: Generation of Reporters by Incorporating Noble Metals

To ensure the bio-safety, sensitivity, and accuracy of AVEC used in vivo as described herein, a stable link between AVEC and a reporter molecule such as a noble metal atom was accomplished by designing and engineering various metal binding domains (MBD).

They include binding domains of noble metals (e.g., Au, Ag, Pd, Pt) and paramagnetic and/or their salts (e.g., Gd, Eu, Fe, Co, Ni, iron oxides, or other suitable metals) and for nanoparticles assembled into the core-shell suitable for EDX, XRTFS, CT, Mass Spec., are listed below where n: 0-1000:

MAP16-B
(SEQ ID NO: 336)
(Gly-)n-CyS
(SEQ ID NO: 337)
(Gly-Arg-)n-Cys
(SEQ ID NO: 338)
(Gly-Lys-)n-CyS
(SEQ ID NO: 339)
(Gly-Asp-Gly-Arg)n-Cys
(SEQ ID NO: 340)
(Gly-Glu-Gly Arg)n-Cys
(SEQ ID NO: 341)
(Gly-Asp-Gly-Lys)n-Cys
(SEQ ID NO: 342)
(Gly-Glu-Gly-Lys)n-CyS

Gd or Eu binding domains suitable for MRI and NMR, as well as MACS are listed below:

(SEQ ID NO: 343)
(Glu-Glu-Glu-Glu-Glu)n
(SEQ ID NO: 344)
(Glu-Glu-Glu-Glu-Glu-Glu)n
(SEQ ID NO: 345)
(Asp-Asp-Asp-Asp-AsP)n
(SEQ ID NO: 346)
(Asp-Asp-Asp-Asp-Asp-AsP)n
(SEQ ID NO: 347)
Phe-His-Cys-Pro-Tyr-Asp-Leu-Cys-His-Ile-Leu
Ni and Co binding domains aa:
(SEQ ID NO: 348)
(Gly-Asp-Gly-Arg)n-(His)5,6
(SEQ ID NO: 349)
(Gly-Glu-Gly Arg)n-(His)5,6
(SEQ ID NO: 350)
(Gly-Asp-Gly-Lys)n-(His)5,6
(SEQ ID NO: 351)
(Gly-Glu-Gly-Lys)n-(His)5,6
(SEQ ID NO: 352)
(Gly-Arg-)n-(His)5,6
(SEQ ID NO: 353)
(Gly-Lys-v-(His)5,6

Beckman BIOMEK FX Span-8 and 96 Channel Robotic System was loaded with each of the domains within a separate channel. In particular one of the channels contained the noble metal nanoparticles (e.g., gold) or superaparamagnetic, or core shell nanoparticles. Each of these domains contained metal binding domain at the amino or carboxyl terminus as detailed below. The sequence of the processing allowed addition of the single domain to a single particle at a time. Alternatively, a microfluidic system was used with the identical aim. As a result, heterospecific mono-, di-, tri-, poly-mer scFv, sdFv, CDR AVEC domains were easily assembled and tested, while firmly anchored to the nanoparticles as the core structure. Some constructs led to expression of fusion proteins, but their MBD at the carboxyl or amino terminus served as the anchors to the nanoparticles.

Manufacturing of Pure Noble Metal Nanoparticles.

Nanoparticles derived from noble metals Au, Pt, Pd and Ag were generated by laser ablation of 99.99% purity metal foils in a chamber filled with deionized water under continuous flow as described previously [21, 22]. Some variability in sizes was compensated by gradient ultracentrifugation, which also resulted in their condensation.

Reporters with Noble Metals and Guided by Targeting Domains.

Plasmid constructs were generated as described previously [21, 22]. Briefly, molecular probe constructs having coding sequences comprising CDR, scFv, sdFv, CD anti-ssDNA and/or anti-dsDNA (i.e., AVEC marker binding domain) as fusions with MBD were selected from surface display libraries as described above.

The extended coding sequences were then cloned into pM vectors designed with the following: CMV immediate early promoter, SV40 poly(A) termination, and neomycin-resistance. Constructs for these scFv were then electroporated into human myelomas for expression of the scFv. The myelomas were cultured in modified roller bottles according to protocols published in the details [21, 22]. Expression of the constructs by the myeloma resulted in the production and secretion of scFv, sdFv, CDR, CD, IgM, IgG, Fab.

Alternatively, selection of molecular probe constructs were conducted via in vitro evolution involving phage display, yeast display, myeloma display, and/or ribosomal display. The selection method had no implication for the choice of expression, which was conducted in CHO and HEK293 cells according to established protocols. Alternatively, cell free expression systems were used according to the standard protocols.

Chelating sites fused with AVEC marker binding domains were then covalently bound to gold nanoparticles to form gold-linked AVEC. While the current examples provide for the production of gold nanoparticles, nanoparticles using other noble metals (e.g., Pt, Pd, Ag) may be successfully manufactured according to previously developed methods well known to the technicians skilled in the art [21, 22]. Purification of the gold-charged AVEC was accomplished using affinity and size exclusion chromatography columns.

Determination of Noble Metal Atoms Per Nanoparticle and Number of Nanoparticles Per AVEC.

The number of atoms per nanoparticle was determined by measuring the diameter with FEEFTEM (Titan) or EFTEM (LE0912) or FESTEM (HB501) at zero loss followed by measuring MDN with EDX and/or EELS of the beam parked over the nanoparticle using the Si drifted detector or ccd chip (Noran, Zeiss or Gatan, respectively). The ratios of nanoparticles to scFv, sdFv, CDR, CD, IgM, IgG, Fab was determined by ratios between the noble metal nanoparticle and carbon counts from EDX and EELS in Zeiss 912 or Titan or VG equipped with Zeiss or Gatan software.

### Example 8. Generation of Reporters by Incorporating Superparamagnetic Molecules

To ensure the bio-safety, sensitivity, and accuracy of the AVEC reporters in vitro and in vivo using nuclear magnetic resonance techniques as described herein, a stable link between AVEC marker targeting domain and a reporter molecule such as a superparamagnetic atom was accomplished by designing and engineering various specific metal binding domains (MBD). [22]

Plasmid constructs were described above and in the peer-reviewed articles. Coding sequences for ssDNA and dsDNA were selected from the surface displayed libraries cloned into pM vectors designed with CMV immediate early promoter, SV40 poly(A) termination, and neomycin-resistance. The constructs were electroporated or lipofected into human myelomas, CHO and/or HEK293. Expression of these constructs resulted in the secretion of ready fusion proteins. In some cases, these proteins were exposed to a couple of rounds of de- and re-naturation processes by exposing them to high pressure freezing at 3000 mbar, −196 deg C. Chelating sites were saturated with metal ions: Gd, Eu, Fe, Ni and Co. Alternatively, the iron oxide nanoparticles were coated with shells of noble metals. They were linked to fusion proteins involving protocols identical to those as used for noble metals. Purification from non-bound metal was performed on affinity columns. The myelomas were cultured in modified roller bottles (Sigma) or bioreactors (New Brunswick) according to standard protocols. Alternatively, cell free expression systems were used according to standard protocols.

Determination of Metal Atoms Incorporated into Chelating Sites.

The chelating sites of MBD were saturated with Gd or other superparamagnetic ions. Subsequently, these samples were purified on the affinity columns. Finally, they were analyzed with electron energy loss spectral imaging (EELS) and x-ray dispersive spectroscopy to determine total C to Gd metal atom ratio or in other words, the number of incorporated atoms per AVEC molecule.

Alternatively, the AVEC were altered through amine or carboxyl terminus modification with Iodine. Subsequently, these samples were purified on the gels. They were analyzed using ratios between Iodine and Carbon using EDX and EELS.

Alternatively, the AVEC were altered through amine or carboxyl terminus modification involving insertion of MBD and linked with noble gold metal clusters. Subsequently, these samples were purified on the size exclusion chromatography columns. They were analyzed using ratios between I and C using EDX and EELS.

### Example 9: Generation of Reporters by Incorporating Fluorochromes

Bioluminescent and fluorescent compounds or molecules and dyes that may be used as reporter components in accordance with the embodiments of the disclosure include, but are not limited to, fluorescein, fluorescein isothiocyanate (FITC), Oregon Green™, rhodamine, Texas red, tetrarhodimine isothiocynate(TRITC), Cy3, Cy5, etc.), fluorescent markers (e.g., green fluorescent protein (GFP), phycoerythrin, etc.), autoquenched fluorescent compounds that are activated by tumor associated proteases, enzymes (e.g., luciferase, horseradish peroxidase, alkaline phosphatase, etc.), nanoparticles, biotin, digoxigenin, fluorescent metals including, but not limited to Eu, Tb, Ru, fluorescent amino acids (e.g., Tyrosine), or combination thereof.

According to some embodiments described herein, a fluorescent reporter may be used to measure by flow cytometry (FCM) and/or sort cells targeted by the AVEC described herein using fluorescent flow cytometry methods known in the art including, but not limited to, fluorescence activated cell sorting (FACS). Enzymes that may be used as reporter components in accordance with the embodiments of the disclosure include, but are not limited to, horseradish peroxidase, alkaline phosphatase, acid phosphatase, glucose oxidase, beta.galactosidase, beta.glucoronidase or beta.lactamase. Such enzymes may be used in combination with a chromogen, a fluorogenic compound or a luminogenic compound to generate a detectable signal.

According to some embodiments, the AVEC reporter binding domain provide a binding site for the reporter compound. The reporter binding domain may be a metal binding domain (MBD), a chelating site or an organic functional group (e.g., amino, carboxyl, thiol or azide groups) or a synthetic chelate (e.g., DTPA or DOTA). For example, when the reporter component is a metal (e.g., a noble metal or superparamagnetic metal) or paramagnetic ion, the AVEC may include a metal binding domain. In such case, the reporter component may be reacted with a reagent having a long tail with one or more chelating groups attached to the long tail for binding these ions. The long tail may be a polymer such as a polylysine, polysaccharide, or other derivatized or derivatizable chain having pendant groups to which may be bound to a chelating group for binding the ions. Examples of chelating groups that may be used according to the disclosure include, but are not limited to, ethylenediaminetetraacetic acid (EDTA), EGTA, diethylenetriaminepentaacetic acid (DTPA), DOTA, NOTA, NETA, TETA, porphyrins, polyamines, crown ethers, bisthiosemicarbazones, polyoximes, and like groups. The chelate is normally linked to the antibody or functional antibody fragment by a group which enables formation of a bond to the molecule with minimal loss of immunoreactivity and minimal aggregation and/or internal crosslinking. The same chelates, when complexed with nonradioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium, gadolinium, iodine, and copper, respectively. In certain embodiments, chelating moieties may be used to attach a PET imaging agent, such as an sup.18F complex, to a targeting molecule for use in PET analysis.

According to some embodiments, a metal binding domain (MBD) that is part of AVEC described herein may include, but is not limited to, the listed sequences, or any functional fragment thereof:

TABLE
(Gly)nCys (SEQ ID NO: 336);
(GlyArg)nCys (SEQ ID NO: 337);
(GlyLys)nCyS (SEQ ID NO: 338);
(GlyAspGlyArg)nCys (SEQ ID NO: 339);
(GlyGluGlyArg)nCys (SEQ ID NO: 340);
(GlyAspGlyLys)nCys (SEQ ID NO: 341);
(GlyGluGlyLys)nCys (SEQ ID NO: 342);
MAP16B;
(GluGluGluGluGlu)n (SEQ ID NO: 343);
(GluGluGluGluGluGlu)n (SEQ ID NO: 344);
(AspAspAspAspAsp)n (SEQ ID NO: 345);
(AspAspAspAspAspAsp)n (SEQ ID NO: 346);
PheHisCysProTyrAspLeuCysHisIleLeu (SEQ ID NO: 347);
(GlyAspGlyArg)n(His)5,6 (SEQ ID NO: 348);
(GlyGluGlyArg)n(His)5,6 (SEQ ID NO: 349);
(GlyAspGlyLys)n(His)5,6 (SEQ ID NO: 350);
(GlyGluGlyLys)n(His)5,6 (SEQ ID NO: 351);
(GlyArg)n(His)5,6 (SEQ ID NO: 352);
(GlyLysv(His)5,6 (SEQ ID NO: 353).

### Example 10: Validation of AVEC Antibody Domains' Specificity and Sensitivity by Nuclear Magnetic Resonance

The following materials and methods are used for the validation experiments described herein, but also apply to the experiments described in Examples below as reported in FIG. 2A, FIG. 5A, FIG. 5B.

### Results.

The primers used for amplification are listed below.

TABLE

| | OLIGO | length | t melting | gc% | any | 3′ | seq |
|---|---|---|---|---|---|---|---|
| ERBB1 | LEFT PRIMER | 20 | 60.01 | 50.00 | 6.00 | 1.00 | cagcgctaccttgtcattca |
| | | | | | | | (SEQ ID NO: 1) |
| | RIGHT PRIMER | 20 | 60.00 | 55 00 | 7.00 | 2.00 | tgcactcagagagctcagga |
| | | | | | | | (SEQ ID NO: 2) |
| | HYB OLIGO | 20 | 60.08 | 45 00 | 800 | 3.00 | gaatgcatttgccaagtcct |
| | | | | | | | (SEQ ID NO: 3) |
| | OLIGO | len | tm | gc% | any | 3′ | seq |
| ERBB2 | LEFT PRIMER | 20 | 59.99 | 55.00 | 2.00 | 0.00 | ccataacacccacctctgct |
| | | | | | | | (SEQ ID NO: 19) |
| | RIGHT PRIMER | 20 | 59.95 | 55 00 | 600 | 3.00 | actggctgcagttgacacac |
| | | | | | | | (SEQ ID NO: 20) |
| | HYB OLIGO | 20 | 60.06 | 55.00 | 4.00 | 1.00 | accaagctctgctccacact |
| | | | | | | | (SEQ ID NO: 39) |
| | OLIGO | len | tm | gc% | any | 3′ | seq |
| ERBB3 | LEFT PRIMER | 20 | 59.95 | 60 00 | 3.00 | 3.00 | gagcccagaggagaagact |
| | | | | | | | (SEQ ID NO: 67) |
| | RIGHT PRIMER | 20 | 59.99 | 55 00 | 600 | 0.00 | tctgatgcgacagacactcc |
| | | | | | | | (SEQ ID NO: 68) |
| | HYB OLIGO | 20 | 59.83 | 60.00 | 3.00 | 0.00 | gagtctgagtgttcggaggg |
| | | | | | | | (SEQ ID NO: 69) |
| | OLIGO | len | tm | gc% | any | 3′ | seq |
| ERBB4 | LEFT PRIMER | 20 | 60.04 | 45.00 | 3.00 | 0.00 | tttcgggagtttgagaatgg |
| | | | | | | | (SEQ ID NO: 85) |
| | RIGHT PRIMER | 20 | 59.97 | 50.00 | 7.00 | 2.00 | gaaactgtttgccccctgta |
| | | | | | | | (SEQ ID NO: 86) |
| | HYB OLIGO | 20 | 60.04 | 50.00 | 4.00 | 4.00 | aagatggaagatggcctcct |
| | | | | | | | (SEQ ID NO: 87) |

We measured sensitivity of detection of HER-2 on cancer cells by labeling cells with superparamagnetic antibodies and measuring relaxivities by nuclear magnetic resonance (NMR) (FIG. 2A, FIG. 5A). For this purpose, the SK-BR-3, MCF-7, patients' breast cancer cells, and human breast epithelial cells were labeled with trastuzumab, anti-HER-2₀₀₁, anti-HER-2₀₀₄, anti-HER-2₀₀₁×HBsAg, and anti-HER-2₀₀₄×HBsAg and anti-HBsAg. As the control, the MCF-7 cells, which do not overexpress HER-2, were labeled with the same antibodies. As the control, these cells were also labeled with the isotype antibodies. The measurements revealed statistically significant differences between the HER-2+SK-BR-3 and patients' HER-2+ breast cancer cells, which were heavily labeled with therapeutics: trastuzumab, anti-HER-2₀₀₁, anti-HER-2₀₀₄, anti-HER-2₀₀₁×HBsAg, and anti-HER-2₀₀₄×HBsAg versus the HER-2-MCF-7 and human breast epithelial cells, which were practically not labeled. These measurements revealed also statistical differences between these therapeutics and the isotypes. Therefore, we validated efficient targeting of the HER-2+SK-BR-3 and the patients' HER-2+ breast cancer cells (the measurements included are representative for all 10 patients' biopsies studied) breast cancer cells by the AVEC: anti-HER-2_{—004}×HBsAg and AVEC: anti-HER-2_{—001}×HBsAg.

Breast and ovarian cancer cells were grown and treated as above. Specificity and sensitivity were validated in NMR. For this purpose, the cells were labeled with superparamagnetic reporters.

Labeling of cancer cells with AVEC having magnetic reporters changed their properties, while making them susceptible to magnetic field. The more cells were labeled, the higher relaxivity and brighter signal

### Example 11: Validation of AVEC Antibody Domains' Specificity and Sensitivity by Flow Cytometry

### Results.

We determined specificity and sensitivity of the HER-2 domains' targeting by flow cytometry (FIG. 2D, FIG. 2E). Moreover, we performed tests of cross-blocking as shown in Table 1. While labeling with AVEC: anti-HER-2₀₀₁×HBsAg interfered on the statistically significant level with trastuzumab, labeling with AVEC: anti-HER-2₀₀₄×HBsAg did not. Therefore, we concluded that these antibodies: trastuzumab and AVEC: anti-HER-2₀₀₁×HBsAg target the same, but AVEC: anti-HER-2₀₀₄×HBsAg the different domains on the HER-2 receptors.

TABLE 1
Cross-blocking of AVEC: anti-HER-2

| | trastuzumab | anti-HER-2001 | anti-HER-2004 | anti-HER- |
|---|---|---|---|---|
| trastuzumab | + | + | − | + |
| anti-HER- | + | + | − | + |
| 2001 | | | | |
| anti-HER- | − | − | + | − |
| 2004 | | | | |
| anti-HER- | + | + | − | + |
| anti-HER- | − | − | + | − |
| anti-HBsAg | − | − | − | − |

### Example 12: Validation of AVEC Antibody Domains' Specificity and Sensitivity by Immunoblotting and Immunoprecipitation

The cells and tissues were either frozen crushed in the rapid controlled rate freezer or native disintegrated with ultrasonicator (Branson Ultrasonic, Danbury, Conn., USA). After being homogenized within the sample buffer they were either stored in liquid nitrogen or lyophilized.

Alternatively, the targeted molecules on cancer cells were expressed after amplification of the coding sequences for the targeted receptors using the primers listed below in the expression systems described above for the antibodies.

They were electrophoresed in the native buffer (Invitrogen, Carlsbad, Calif., USA). They were vacuum- or electro transferred onto the PVDF membranes (Amersham, Buckinghamshire, UK, EU). The membranes carrying the transferred proteins were first soaked within human serum and thereafter labeled with the bioengineered, biosimilar, and referenced anti-HER-2 antibodies. The anti-HBsAg isotype antibodies served as the controls. The images of the blots were acquired and quantified with Fluoroimager (Molecular Dynamics, Sunnyvale, Calif., USA) or Storm 840 (Amersham, Buckinghamshire, The anti-HER-2 and anti-HBsAg antibodies were rendered magnetic or fluorescent by conjugating Au coated Fe3O4 nanoparticles or fluorochromes. The sera and liver biopsies' homogenates were mixed with these superparamagnetic antibodies. The targeted molecules rendered fluorescent were pulled out by the means of 1.5T magnet. The intensity of fluorescence was measured on the spectrofluorometer.

### Results.

We measured specificity of targeting of the HBsAg by vaccinated and infected patients' antibodies, while measuring the concentration of this immunogen immunoprecipitated, which was electrophoresed and immunoblotted (FIG. 2B, FIG. 2C). All molecular baits used in this study were pulling out the same 150 kDa molecule, which was recognized by clinical diagnostics for Hepatitis B. By quantitative high gain scanning of the blots, we were able to determine that there were no other molecules pulled out from the sera. The same experiments conducted on the sera of not immunized patients resulted in empty lanes (FIG. 2B, FIG. 2C). Therefore, we concluded that anti-HER-2₀₀₁×HBsAg and anti-HER-2₀₀₄×HBsAg possess high sensitivity and high specificity towards HER-2 and HBV.

### Example 13. Antibody Dependent Redirected Accelerated Amplified Vaccination—Induced—Immunity Therapy (ADRAAVIT) to Inhibit Proliferation

To study collective killing effects of the anti-HER-2 and anti-HER-2×HBsAg upon the breast cancer cells, the patients' cell and serum fraction described below were pooled making erythrocytes-free blood (EFB). Anti-HER-2, anti-HER-2_{—001}×HBsAg, and anti-HER-2₀₀₄×HBsAg were added to the EFB. So were, anti-HBsAg, anti-HPV, anti-HSV, EGFR1, and isotype antibodies as the controls. The incubation with the antibodies continued at the 37° C. incubators. The labeling continued for 1-24 h. It was terminated by washing with the cold buffer. To quantify the numbers of killed cells by flow cytometry (FCM) and fluorescent activated cell sorting (FACS) the samples were stained with propidium iodide (PI) (Sigma-Aldrich, Milwaukee, Wis., USA) used at 50 μg/ml. To determine the numbers of apoptotic cells, they were labeled with anti-phosphatidylserine antibodies.

### Results.

Treatment of the breast cancer cells with increasing concentrations of trastuzumab, anti-HER-2 biosimilars, and the novel AVEC: anti-HER-2₀₀₁×HBsAg biomolecules was followed by pulsing with thymidine marked with tritium. Growth inhibition was calculated as percentage of surviving cells compared to non-treated cells as the control (FIG. 3A). Growth inhibition was attained at much lower concentrations, when the cells were treated with anti-HER-2₀₀₄×HBsAg, over that attained with trastuzumab.

### Example 14. Antibody Dependent Cell Cytotoxicity (ADCC)

To study toxicity to the breast cancer cells caused by the patients' cytotoxic cells—the effectors triggered by the anti-HER-2 antibodies, the peripheral blood mononuclear cells were separated from the blood on Ficoll-Hypaque density gradients. The cells were washed by three cycles of spinning down and suspending in the PBS at pH 7.3. They were rendered fluorescent by adding the stock solution of the DiI membrane dye (Molecular Probes, Inc., Eugene, Oreg., USA) in DMSO for 10 min at 26° C. Small aliquots were washed with the buffer and the cells quantified on FCM as the way to determine the effector to target cells' ratios (ETR). These ratios varied: 10:1, 50:1, and 100:1. Incubations lasted 1-7 h in a 37° C., 5% CO2 incubator.

The numbers of killed cells were determined due to staining with the PI at 50 μg/ml and of surviving cells from the DiO staining counts and thymidine incorporation.

### Results.

Clearly superior efficacy was obtained with AVEC: anti-HER-2₀₀₄×HBsAg, over that attained with trastuzumab. Trastuzumab and both new biomolecular clusters triggered apoptosis (FIG. 3B). The extent of apoptosis progressed rapidly, but the AVEC: anti-HER-2₀₀₁×HBsAg and AVEC: anti-HER-2₀₀₄×HBsAg induced apoptosis in much greater percentage than trastuzumab (˜40%). In all cases, this percentage was statistically much higher than in the MCF-7-HER-2-cells treated on the identical way (FIG. 3C). It was also statistically significantly higher than SK-BR-3 cells treated with the isotype antibody. Trastuzumab and both new biomolecular clusters of anti-HER-2₀₀₁×HBsAg caused necrosis (FIG. 3D). At the initial stages of apoptosis, many cells were only showing outer-membrane display of phosphatidylserine, but were not permeable for PI. At the more advanced stages, they were becoming leaky; thus adding to necrotic counts. Both clusters of anti-HER-2×HBsAg caused massive necrosis of the SK-BR-3 and the patients' HER-2+ breast cancer cells in the percentages, which were statistically significantly much higher over those inflicted by trastuzumab.

### Example 15. Complement Dependent Cytotoxicity (CDC)

To study toxicity to the breast cancer cells caused by the patients' complement system—the effector, the serum was separated by gentle centrifugation from the freshly drawn blood. It was supplemented with the anti-HER-2 and anti-HER-2₀₀₁. Incubations lasted 1-7 h in a 37° C., 5% CO2 incubator. The numbers of killed cells were determined due to staining with the PI at 50 μg/ml and of surviving cells from the DiO staining counts and thymidine incorporation. Superior efficacy of AVEC: anti-HER-2_{—001}×HBsAg and AVEC: anti-HER-2_{—004}×HBsAg is clearly demonstrated in the figure over the naked antibodies. (FIG. 4A)

### Example 16. Factors Affecting Efficacy of AVEC

The processes of breast cancer cells' AVECs are triggered by the specific elements of the patients' immune system: humoral and cellular. We aimed at defining the main factors triggering them. In particular, we were focused on effects of complement concentrations (FIG. 4A) and effector cells to target cells ratios (FIG. 4B). Concentrations of the complement systems' components (CS) determine the patients' ability to fight cancer by complement dependent cytotoxicity (CDC). Concentrations of the complement systems' components in our tests were adjusted within the ranges of healthy adults.

### Results

Our measurements revealed that increasing the concentrations of the C1q and C3 resulted in the statistically significant increase in the efficacy of the HER-2+SK-BR-3 and the patients' HER-2+ breast cancer cells' killing by trastuzumab and anti-HER-2 antibodies as compared to labeling with the isotype antibodies or statistically significantly much higher, when the HER-2+SK-BR-3 and the patients' HER-2+ breast cancer cells were treated with anti-HER-2₀₀₁×HBsAg, and anti-HER-2₀₀₄×HBsAg. Numbers of natural killer cells and cytotoxic lymphocytes in the patient's circulation determine this patient's ability labeling of the HER-2-MCF-7 cells. The efficacy was to execute antibody dependent cell cytotoxicity (ADCC). The numbers of the immune cells were adjusted to clinical lab values. Trastuzumab and our anti-HER-2 biosimilar antibodies caused the cancer cells' AVECs through ADCC already at the ratio of 10:1, but with no statistical difference between them (FIG. 4B). Anti-HER-2₀₀₁×HBsAg, and anti-HER-2₀₀₄×HBsAg inflicted massive AVECs of breast cancer cells, which were at statistically significantly much higher levels, than those inflicted by trastuzumab and anti-HER-2 biosimilars. The isotype antibodies did not have any impact. The MCF-7 cells and the patients' breast epithelial cells did not show signs of responding to these immunotherapeutics in the conditions described. All the measurements were pursued in triplicates. All the data presented are representative for all studied. Treatment with the isotype antibodies did not have any statistically significant impact on the cancer cells' AVEC rates.

### Example 17. Pharmacokinetics, Pharmacodynamics, Pharmacogenomics in Mice and Rats

Mice and rats were acquired from the studies, in which they were considered as surplus and sentenced for euthanasia. They were from either the control group, or from the study group having spontaneous or grafted tumors. The study was carried in the two phases. In the first phase, the blood was drawn through the tail vein and the concentration of the specific circulating vaccine, cancer antigen, and antibodies against them determined using the methods, instruments, and reagents described above (e.g., including but not limited: HBsAg, HBcAg, HBeAg, anti-HBsAg, anti-HBcAg, HER-2, EGFR1, anti-HER-2, anti-EGFR, etc). The mice and rats received AVEC by the vaccine type route of administration at the starting dose and escalated. The aforementioned molecules were measured initially in 24 h, and later weekly intervals. In the second phase, the blood was drawn and the concentration of the specific circulating vaccine, cancer antigen, and antibodies against them determined. The mice and rats received AVEC by the infusion type route of administration with the starting dose and escalated. The molecular imaging was performed as outlined above, while relying on the reporter domains of AVEC. The aforementioned molecules were measured initially in 24 h, and later weekly intervals. There were no adverse effects of the AVEC administration.

### Example 18. Pharmacokinetics, Pharmacodynamics, Pharmacogenomics in Humans

Healthy volunteers are provided with the complete Bill of Rights according to the Declaration of Helsinki. All aspects of safety and absence conflict of interest are brought up. Only if they voluntarily decide to participate, they sign the Patient Informed Consent. The records conceal their identity. The study is carried in the two phases. In the first phase, the blood is drawn and the concentration of the specific circulating vaccine, cancer antigen, and antibodies against them determined the methods, instruments, and reagents described above (e.g., including but not limited: HBsAg, HBcAg, HBeAg, anti-HBsAg, anti-HBcAg, HER-2, EGFR1, anti-HER-2, anti-EGFR, etc). The volunteers receive AVEC by the vaccine type route of administration at the starting dose of 10 micrograms and escalated. The aforementioned molecules are measured initially in 24 h, and later weekly intervals. In the second phase, the blood is drawn and the concentration of the specific circulating vaccine, cancer antigen, and antibodies against them determined. The volunteers receive AVEC by the infusion type route of administration with the starting dose of 10 micrograms and escalated. The aforementioned molecules are measured initially in 24 h, and later weekly intervals.

### Results.

The studies demonstrated the important primary results as the Phase I Clinical Trials presented in FIG. 9. First, there were no serious adverse effects in the sites of injections and infusions of AVEC. Second, the administration by subcutaneous injection resulted in primary lymphatic route of distribution of AVEC. Third, the administration by intravenous infusion resulted in significantly increased half-life of AVEC as compared to naked antibodies.

### Example 19. Statistical Analysis

All the measurements were run in triplicates for each sample from six patients. The numbers were analyzed and displayed using GraphPad software (GraphPad Software, Inc, La Jolla, Calif.). Data were presented as mean of standard error of the mean (SEM). Statistical significance was calculated by t test for two groups.

### Additional Embodiments

In some embodiments, any of the embodiments provided herein can involve a reporting technique(s) that is(are) radiography, CT, MRI, NMR, PET, SPECT, EDX, TRXRF, MassSpec, HPLC, FACS, MACS, FCM, gamma scintigraphy, fluorescence microscopy, electron microscopy, energy filtering transmission electron microscopy, electron energy loss spectroscopy, x-ray dispersion spectroscopy, or Raman spectroscopy.

In some embodiments, any of the embodiments provided herein can involve a reporter(s) that can be used to detect pharmacokinetics of AVEC for research, diagnosis, and therapy, while conducted in vitro, in vivo, ex vivo.

In some embodiments, a utility of AVEC and their methods of manufacturing and administration for predicting efficacy of therapy of patients suffering from cancer comprising detection utilizing AVEC featuring any of the embodiments provided herein.

In some embodiments, a utility of AVEC and methods of their manufacturing and administration for therapy of patients suffering from cancer comprising administration to a patient an effective dose of AVEC utilizing AVEC featuring any of the embodiments provided herein, with the purpose of antibody dependent redirecting, accelerating, amplifying, vaccination-induced immune response (ADRAAVIR) for therapy of cancer.

In some embodiments, a utility of AVEC and methods of their manufacturing and administration for evaluating efficacy of therapy of patients suffering from cancer comprising detection of reporters of any of the embodiments provided herein.

### REFERENCES

The references listed below and all referenced cited above are hereby incorporated in their entirety by reference as if fully set forth herein.
1. Torre L A, Siegel R L, Ward E M, Jemal A (2016) Global cancer incidence and mortality rates and trends—an update. Cancer Epidemiol Biomarkers Prev. 25:16-27
2. Slamon D J, Clark G M, Wong S G, Levin W J, Ulrich A et al (1987) Human breast cancer: correlation of relapse and survival with amplification of the HER-2/neu oncogene. Science 235:177-182
3. Pietras R J, Pegram M D, Finn R S, Slamon D J (1998) Remission of human breast cancer xenografts of therapy with humanized monoclonal antibody to HER-2 receptor and DNA-reactive drugs. Oncogene 17:2235-2249
4. Park J W, Stagg R, Lewis G D, Carter P, Maneval D, Slamon D J, Jaffe H, Shepard H M (1992) Anti-p185HER2 monoclonal antibodies: biological properties and potential for immunotherapy. Cancer Treat Res 61:193-211
5. Figueroa-Magalhães M C, Jelovac D, Connolly R M, Wolff A C (2014) Treatment of HER2-positive breast cancer. Breast 23:128-136
6. http://www.fda.gov/drugs/developmentapprovalporcess/howdrugsaredevelopedandapproved/approvalapplications/therapeuticbiologicapplications/ucm080591.htm
7. http://www.fda.gov/safety/medwatch/safetyinformation/safetyalertsforhumanmedicalproducts/ucm350817.htm; http://www.fda.gov/newsevents/newsroom/pressannouncements/ucm370393.htm
8. Mendes D, Alves C, Afonso N, Cardoso F, Passos-Coelho J L et al (2015) The benefit of HER2-targeted therapies on overall survival of patients with metastatic HER2-positive breast cancer. Breast Cancer Res 17:140-147
9. Sliwkowski M X, Lofgren J A, Lewis G D, Hotaling T E, Fendly B M et al (1999) Nonclinical studies addressing the mechanism of action of Herceptin (Trastuzumab). Semin Oncol 26:60-70
10. Clifford A, Hudis M D (2007) Herceptin—Mechanism of action and use in clinical practice. N Engl J Med 357:39-51
11. Baselga J, Albanell J (2001) Mechanism of action of anti-HER2 monoclonal antibodies. Ann Oncol 12:S35-S41
12. Ahmad S, Gupta S, Kumar R, Varshney G C, Raghava G P (2014) Herceptin resistance database for understanding mechanism of resistance in breast cancer patients. Sci Rep 4:4483
13. Rosenberg S A, Yang J C, Restifo N P (2004) Cancer immunotherapy: moving beyond current vaccines. Nat Med 10:909-915
14. Rosenberg S A (2004) Shedding light on immunotherapy for cancer. N Engl J Med 350:1461-1463
15. Daniels D, Grytdal S, Wasley A (2009). Surveillance for Acute viral hepatitis—United States, 2007. Morbidity and mortality weekly report. Surveillance summaries. 58:1-9. http://www.cdc.gov/mmwr/PDF/ss/ss5803.pdf
16. Wolters G L, Kuijpers L, Kacaki J, Schuurs A (1976) Solid-phase enzyme-immunoassay for detection of hepatitis B surface antigen. J Clin Pathol 29:873-879
17. http://www.fda.gov/biologicsbloodvaccines/vaccines/approvedproducts/ucm398332.htm
18. http://wwwfda.gov/biologicsbloodvaccines/vaccines/approvedproducts/ucm376931.htm
19. SK-BR-3 http://www.atcc.org/Products/All/HTB-30.aspx
20. MCF-7 http://www.atcc.org/Products/All/HTB-22.aspx
21. Malecki M, Hsu A, Truong L, Sanchez S (2002) Molecular immune-labelling with recombinant single-chain variable fragment (scFv) antibodies designed with metal-binding domains. Proc Natl Acad Sci 99:213-218
22. Malecki M, Szybalski W (2012) Isolation of single, intact chromosomes from single, selected ovarian cancer cells for in situ hybridization and sequencing. Gene 493:132-139
23. http://www.imgt.org/3Dstructure-DB/cgi/details.cgi?pdbcode=7637&Part=Chain&Chain=7637L
24. http://www.imgt.org/3Dstructure-DB/cgi/details.cgi?pdbcode=7637&Part=Chain&Chain=7637H
25. Valenzuela P, Gray P, Quiroga M, Zaldivar J, Goodman H M et al (1979) Nucleotide sequence of the gene coding for the major protein of hepatitis B virus surface antigen. Nature 280:815-819
26. Valenzuela P, Medina A, Rutter W J, Ammerer G, Hall B D (1982) Synthesis and assembly of hepatitis B virus surface antigen particles in yeast. Nature 298:347-350
27. Hazra P P, Suriapranata I, Snyder W B, Subramani S (2002) Peroxisome remnants in Pex3 D cells and the requirement of Pex3p for interactions between the peroxisomal docking and translocation subcomplexes. Traffic 3:560-574
28. Kitagawa T, Aikawa T (1976) Enzyme coupled immunoassay of insulin using a novel coupling reagent. J Biochem 79:233-236
29. Heijtink R A, Bergen P, Melber K, Janowicz Z A, Osterhaus A D (2002) Hepatitis B surface antigen (HBsAg) derived from yeast cells used to establish an influence of antigenic subtype (adw2, adr, ayw3) in measuring the immune response after vaccination. Vaccine. 20:2191-2196
30. Scheiblauer H, El-Nageh M, Diaz S, Nick S, Zeichhardt H et al (2010) Performance evaluation of 70 hepatitis B virus surface antigen (HBsAg) assays from around the world by a geographically diverse panel with an array of HBsAg genotypes and HBsAg subtypes. Vox Sang 98:403-414
31. Barbas C F, 3rd, Burton D R, Scott J K, Silverman G J, (2001) Phage Display. CSHLP.
32. Martin A C. (1996) Accessing the Kabat antibody sequence database by computer. Proteins. May; 25(1):130-3.
33. Beerli R R1, Bauer M, Buser R B, Gwerder M, Muntwiler S, Maurer P, Saudan P, Bachmann M F. (2008) Isolation of human monoclonal antibodies by mammalian cell display. Proc Natl Acad Sci USA. 105(38):14336-41.
34. Plummer K A, Carothers J M, Yoshimura M, Szostak J W, Verdine G L. (2005) In vitro selection of RNA aptamers against a composite small molecule-protein surface. Nucleic Acids Res. 33(17):5602-10.
35. Stuve L L, Brown-Shimer S, Pachl C, Najarian R, Dina D, Burke R L. Structure and expression of the herpes simplex virus type 2 glycoprotein gB gene. J Virol. 1987 February; 61(2):326-35.

1. Antibody-vaccine engineered construct(s) (AVEC) comprising of the two main domains [(antibody(ies), vaccine(s)] and the additional domain(s) [(linker(s), reporter(s)].

2. Antibody of the AVEC of the claim 1, is the AVEC antibody domain, is (are) the AVEC guiding and immune effector domain(s).

3. Vaccine of the AVEC of the claim 1, is the AVEC vaccine domain, is (are) the AVEC vaccine domain(s) eliciting immune response t vaccine.

4. Linker(s) of the AVEC of the claim 1, is (are) the AVEC domain linking antibody(ies) of the claim 1 with vaccine(s) of claim 1.

5. Reporter(s) of the AVEC of the claim 1, is (are) the AVEC domain(s) reporting presence and/or localization of the AVEC of the claim 1.

6. Antibody(ies) of claim 1, wherein the antibody(ies) is (are) comprising antibodies including, but not limiting to an IgG, IgM, IgA, IgE, IgD antibody, Fab fragment(s) of those, Fab2 fragment(s) of those, single chain variable fragment(s) (scFv), dual chain variable fragment(s) (dcFv), single domain variable fragment(s) (sdFv), single domain antibody *SDA), CDR(s), affibody, aptamer and alike including, but not limited to all outlined below including their mutants:
A Antibody(ies) of claim 1, wherein the antibody is (are) comprising Anti-HER-2;
B Antibody(ies) of claim 1, wherein the antibody is (are) comprising Anti-EGFR-1;
C Antibody(ies) of claim 1, wherein the antibody is (are) comprising Anti-CD20;
D Antibody(ies) of claim 1, wherein, the antibody is (are) comprising Anti-CD-52;
E Antibody(ies) of claim 1, wherein the antibody is (are) comprising Anti-CD-44;
F Antibody(ies) of claim 1, wherein the antibody is (are) comprising Anti-PSMA;
G Antibody(ies) of claim 1, wherein the antibody is (are) comprising Anti-CTLA-4;
H Antibody(ies) of claim 1, wherein the antibody is (are) comprising Anti-EpCAM;
I Antibody(ies) of claim 1, wherein the antibody is comprising Anti-SSEA-4;
J Antibody(ies) of claim 1, wherein the antibody is (are) comprising Anti-SSEA-3;
K Antibody(ies) of claim 1, wherein the antibody is (are) comprising Anti-TRA-160;
L Antibody(ies) of claim 1, wherein the antibody is (are) comprising Anti-TRA-181;
M. An antibody is anti-CD47 antibody; or
N. An antibody is anti-PD-L1 antibody.

7. Vaccine of claim 1, wherein the vaccine is (are) comprising of VLP, attenuated, denatured vaccines and alike including, but not limited to all as outlined below:
A Vaccine(s) of claim 1, wherein the vaccine is (are) comprising HBV
B Vaccine(s) of claim 1, wherein the vaccine is (are) comprising HCV
C Vaccine(s) of claim 1, wherein the vaccine is (are) comprising HAV
D Vaccine(s) of claim 1, wherein the vaccine is (are) comprising HEV
E Vaccine(s) of claim 1, wherein the vaccine is (are) comprising HPV
F Vaccine(s) of claim 1, wherein the vaccine is (are) comprising EBV
G Vaccine(s) of claim 1, wherein the vaccine is (are) comprising CMV
H Vaccine(s) of claim 1, wherein the vaccine is (are) comprising VZV
I Vaccine(s) of claim 1, wherein the vaccine is (are) comprising HSV
J Vaccine(s) of claim 1, wherein the vaccine is (are) comprising Influenza
K Vaccine(s) of claim 1, wherein the vaccine is (are) comprising HIV
L Vaccine(s) of claim 1, wherein the vaccine is (are) comprising Polio
M Vaccine(s) of claim 1, wherein the vaccine is (are) comprising Mumps
N Vaccine(s) of claim 1, wherein the vaccine is (are) comprising Measles
O Vaccine(s) of claim 1, wherein the vaccine is (are) comprising Rotavirus
P Vaccine(s) of claim 1, wherein the vaccine is (are) comprising Dengue
Q Vaccine(s) of claim 1, wherein the vaccine is (are) comprising Zika
R Vaccine(s) of claim 1, wherein the vaccine is (are) comprising Chikungunya
S Vaccine(s) of claim 1, wherein the vaccine is (are) comprising Yellow fever
T Vaccine(s) of claim 1, wherein the vaccine is (are) comprising Haem.infl.
U Vaccine(s) of claim 1, wherein the vaccine is (are) comprising Rubella
V Vaccine(s) of claim 1, wherein the vaccine is (are) comprising Diphtheria
W Vaccine(s) of claim 1, wherein the vaccine is (are) comprising Pertussis
X Vaccine(s) of claim 1, wherein the vaccine is (are) comprising Tetanus
Y Vaccine(s) of claim 1, wherein the vaccine is (are) comprising Tuberculosis
Z Vaccine(s) of claim 1, wherein the vaccine is (are) comprising Cholera
AA Vaccine(s) of claim 1, wherein the vaccine is (are) comprising Anthrax
BB Vaccine(s) of claim 1, wherein the vaccine is (are) comprising Botulisms

8. Linker of claim 1, wherein the chemical reagent is engineered between the main domains to link them together and alike including, but not limited to all as outlined below, which is (are) comprising:
A Linker of claim 1, which is (are) comprising SMBC
B Linker of claim 1, which is (are) comprising SMCC
C Linker of claim 1, which is (are) comprising EDC
D Linker of claim 1, which is (are) comprising PTAD
E Linker of claim 1, which is (are) comprising BMC
F Linker of claim 1, which is (are) comprising NHS esters
G Linker of claim 1, which is (are) comprising isothiocyanates
H Linker of claim 1, which is (are) comprising benzoyl fluorides
I Linker of claim 1, which is (are) comprising maleimides
J Linker of claim 1, which is (are) comprising iodoacetamides
K Linker of claim 1, which is (are) comprising thiopyridines
L Linker of claim 1, which is (are) comprising arylopropiolonitrile
M Linker of claim 1, which is (are) comprising diazonia
Linker of claim 1, wherein the amino acid(s) (AA) (one letter AA code)
is(are) engineered between the main domains to link them together by means of bifunctional linkers and alike including, but not limited to all as outlined below, which is (are) comprising:
A Linker of claim 1, which is (are) comprising {SmSnSoGp}q
B Linker of claim 1, which is (are) comprising PEG 0-100 kDa
C Linker of claim 1, which is (are) comprising 6-aminohexanoic acid: H2N—(CH2)p-COOH
D Linker of claim 1, which is (are) comprising H2n-(CH2)q-NH2
E Linker of claim 1, which is (are) comprising COOH—(CH2)r-NH2
F Linker of claim 1, which is (are) comprising COOH—(CH2)s-COOH
G Linker of claim 1, which is (are) comprising COOH—(CH2)t-SH
H Linker of claim 1, which is (are) comprising SH—(CH2)u-NH2
Where p,q,r,s,t,u=0-1000

9. Linker of claim 1, wherein the AA(s) is(are) engineered between the main domains to link them together by means of expressed coding sequence(s) of DNA that codes for linker as means of creating fusion protein with recombinant protein(s) linker(s) and alike including, but not limited to all as outlined below, which is (are) comprising

10. The linker of claim 1, which is (are) comprising DNA sequences coding for AA of claim 8

11. Reporter of claim 1, wherein reporter is (are) including, but not limited to all as outlined below engineered at the AVEC termini or between the main domains to report presence and/or localization of AVEC.

12. The AVEC of claim 11, wherein the AVEC reporting domain is a metal nanoparticle.

13. The reporter of claim 11, wherein the metal nanoparticle is selected from the group consisting of:
A Au,
B Pt,
C Pd,
D Ag.

14. The reporter of claim 11, wherein the magnetic ion or nanoparticle.

15. The reporter of claim 11, wherein superparamagnetic metal is:
A Gd,
B Eu,
C Fe,
D Ni,
E Co.

16. The reporter of claim 11, wherein the metal nanoparticle tag is a core-shell nanoparticle, the core shell nanoparticle comprising an inner superparamagnetic metal core and an outer noble metal shell.

17. A method of treating breast cancer, the method comprising administering the antibody-vaccine engineered construct(s) (AVEC) of claim 2 to a subject in need thereof.

18. The reporter of claim 11 including but not limiting:
A Tc99m,
B I125,
C I131,
D F18,
E Cu64.

19. The reporter of claim 11, wherein is an ion or nanoparticle selected from the group consisting of fluorochromes (aka fluorescent dyes, fluorophores).

20. The reporter of claim 19 including, but not limiting:
A FITC,
B TRITC,
C rhodamine(s),
D Texas Red,
E indocyanin green,
F phycoerythrins,
G Alexa(s),
H BODIPY,
I DRAQ,
J CYTRAK,
K coumarins,
L Pacific(s),
M Cy(s),
N IRD(s),
O Green fluorescent proteins and their modifications.
